(19) United States
(12) Patent Application Publication
(10) Pub. No.: US 2024/0150820 A1
(43) Pub. Date: May 9, 2024

(54) ASSESSMENT OF BIOLOGICAL PARTICLES IMMOBILIZED IN A HYDROGEL

(71) Applicant: CHEMOMETEC A/S, Allerød (DK)

(72) Inventors: Rune TROELSGAARD PEDERSEN, Vanløse (DK); Søren KJÆRULFF, Hillerød (DK); Alfred HANSWILLEMENKE, Dyssegård (DK)

(21) Appl. No.: 18/549,611

(22) PCT Filed: Mar. 11, 2022
(86) PCT No.: PCT/EP2022/056361

§ 371 Date: Sep. 8, 2023

(30) Foreign Application Priority Data
Mar. 11, 2021 (EP) 21162018.2
Jul. 8, 2021 (EP) 21184493.1
Oct. 5, 2021 (EP) 21201030.0

(51) Int. Cl. C12Q 1/6844; C12Q 1/6834

(52) U.S. Cl. CPC C12Q 1/6846; C12Q 1/6834

(57) ABSTRACT

Detection and analysis of biological particles can provide useful information with respect to a wide range of applications. Those include but are not limited to analysis of disease markers in diagnostic samples, quantification of bacteria and/or viral particles in environmental samples, and detection of specific proteins in cell culture samples. The methods presented herein provide simple, rapid and reliable means of detecting and analyzing biological particles in a hydrogel, and are especially suitable for quantification of for example nucleic acids, viral particles, bacteria, yeast, animal cells, and proteins. The invention also relates to a kit of parts and to a system for detection and analysis of biological particles in a hydrogel.

### TECHNICAL FIELD

The present invention relates to detection and analysis of biological particles in a hydrogel. The methods presented herein provide simple, rapid and reliable means of detecting and analyzing biological particles, and are especially suitable for quantification of for example nucleic acids, viral particles, bacteria, yeast, animal cells, and proteins. The invention also relates to a kit of parts, and to a system for use, for detection and analysis of biological particles in a hydrogel.

### BACKGROUND

Detection and analysis of biological particles can provide useful information with respect to a wide range of applications. Those include but are not limited to analysis of disease markers in diagnostic samples, quantification of bacteria and/or viral particles in environmental samples, and detection of specific proteins in cell culture samples. However, current methods are often complex and time consuming. In particular, several of the current methods, such as flow cytometry and real time PCR (qPCR), require expensive equipment and trained personnel.

Isothermal amplification is a low-cost nucleic acid amplification technique which due to its simplicity and ruggedness provides major advantages over other amplification technologies. Compared to conventional PCR, no expensive laboratory equipment, hereunder thermal cyclers, are needed for conducting the amplification reaction. Other advantages include less sensitivity to inhibitors which may be present in complex samples, and high specificity, as it can amplify a specific gene by discriminating a single nucleotide difference. Isothermal amplification can also be coupled with reverse transcription, enabling detection of RNA.

During isothermal amplification, the target sequence is amplified at a constant temperature using specialized polymerases. The reaction product, i.e. the amplified nucleic acid, can for example be detected by the naked eye, by observing turbidity observed from the reaction precipitate, or by fluorescence by using fluorescent probes and intercalating dyes. However, as the isothermal reaction is normally run as an all-in-one-pot reaction, the assay provides only qualitative and no quantitative information. In contrast, conducting isothermal amplification in a hydrogel enables quantification of the amount and/or the concentration of the biological particles which are analyzed. The reaction product, i.e. the amplified nucleic acid, can be detected as spots with unaided eyes or using a microscope and/or other equipment, depending on the labelling of the reaction product.

Hydrogel-based isothermal amplification has been used for detection and quantification of coliphage MS2 (Huang et al., 2018). The authors used loop-mediated isothermal amplification (LAMP) to quantify the amount of MS2 phage in wastewater samples. Two different types of hydrogels were used for the assays; a polyacrylamide (PA) and a polyethylene glycol (PEG) hydrogel, formed via chemical catalysis and Michael addition, respectively. In a similar approach, LAMP of Salmonella typhi DNA was demonstrated in PEG gelbeads (Zhu et al., 2019).

### SUMMARY

The present invention offers a simple, effective and reliable method for detection and analysis of biological particles in a hydrogel using nucleic acid amplification, such as isothermal amplification.

The inventors have developed a method where biological particles can be analyzed in a hydrogel, and where curing of said hydrogel is inducible and thus controllable. Inducible curing of the hydrogel provides several advantages compared to current techniques. For example, the overall processing time is decreased, and the effect on the biological particles to be analyzed is lower, as the time of the curing step is decreased compared methods where chemical curing is used. Further, the sample can remain liquid until it has been loaded into a slide or a sample compartment and curing is induced. Finally, imaging of the sample can be initiated at once when curing has been initiated.

The inventors have further developed a method for implementing a pattern, i.e. creating compartments, in the hydrogel by illuminating the hydrogel with a light pattern to induce gelling in illuminated parts of the hydrogel. Thus, they have developed a method to efficiently generate a number of small wells in the hydrogel, wherein an array of samples may be subjected to any type of reaction, such as for example nucleic acid amplification, for example isothermal amplification, in a multiplex mode. The biological particles can thus remain in a substantially liquid environment during analysis while being separated from other compartments by the at least substantially gelled walls. One preferred advantage of having the biological particles in a substantial liquid environment is that any component of the isothermal amplification process is in a liquid environment, thus allowing faster movement of for example isothermal amplification reagents toward the biological particle, than is generally possible in the solid phase of the hydrogel, which in turn increases rate of product formation. Furthermore, the adaption of chemical and biological assays that take place in a liquid to a gel phase, requires more optimization than adaption of chemical and biological assays that take place in a liquid into a liquid phase surrounded by a gel phase.

Thus, herein is provided a method of creating compartments in a sample with biological particles, said method comprising the steps of:
a. mixing the sample with curable polymer hydrogel reagents, the curable polymer hydrogel reagents comprising a photoinitiator;
b. illuminating the sample with a light pattern to induce gelling in illuminated parts thereby creating a hydrogel with a number of substantially liquid compartments separated by substantially gelled parts.

Further provided herein is a kit for detecting a biological particle in a sample, said kit comprising:
a. polymer hydrogel reagents; and
b. additional reagents, the reagents being capable of generating a reaction product in the vicinity of particles having a specific target.

Also provided herein is a system for detecting a biological particle in a sample, said system comprising:
a. a holder for a cassette with a sample compartment,
b. image forming means capable of forming an image of at least part of the sample in the sample compartment on image acquisition means,
c. image processing means,
d. at least one illumination source capable of illuminating the sample in the sample compartment with a light pattern to induce gelling in illuminated parts thereby creating a hydrogel with a number of substantially liquid compartments separated by substantially gelled parts; and
e. thermostatically controlled heating means capable of heating and optionally cooling the sample in the sample compartment.

Further provided herein is a method of detecting and/or analyzing a biological particle in a sample, said method comprising the steps of:
a. mixing the sample with polymer hydrogel reagents and nucleic acid amplification reagents, such as isothermal amplification reagents, the amplification reagents being capable of generating a reaction product in the vicinity of particles having a specific target;
b. inducing curing of the mixture to a hydrogel;
c. incubating the mixture or hydrogel, whereby one or more reaction products are produced as a result of the nucleic amplification reaction;
d. recording one or more images of the hydrogel.

Also provided herein is a method of detecting and/or analyzing a biological particle in a sample, said method comprising the steps of:
a. mixing the sample with curable polymer hydrogel reagents, the curable polymer hydrogel reagents comprising a photoinitiator;
b. illuminating the sample with a light pattern to induce gelling in illuminated parts thereby creating a number of substantially liquid compartments separated by substantially gelled parts;
c. detecting or analyzing particles in the substantially liquid compartments.

Further provided herein is a method for detecting or analyzing extrachromosomal DNA (ecDNA) and/or extrachromosomal circular DNA (eccDNA), the method comprising the steps of:
a. mixing nucleic acid amplification reagents, such as isothermal amplification reagents, curable polymer hydrogel reagents, and sample comprising ecDNA and/or extrachromosomal circular DNA (eccDNA);
b. curing the mixture to a hydrogel;
c. incubating the mixture or hydrogel, whereby one or more amplicons are produced as a result of the amplification reaction amplifying whole or part of the ecDNA and/or extrachromosomal circular DNA (eccDNA), if said ecDNA and/or extrachromosomal circular DNA (eccDNA) is present in the sample;
d. recording one or more images of the hydrogel.

Also provided herein is a method for detecting and/or analyzing viral particles in a sample, the method comprising the steps of:
a. mixing nucleic acid amplification reagents, such as isothermal amplification reagents, curable polymer hydrogel reagents, and sample comprising viral particles;
b. curing the mixture to a hydrogel;
c. incubating the mixture or hydrogel, whereby one or more amplicons are produced as a result of the amplification reaction amplifying the nucleic acids present in the viral particle, if said viral particle is present in the sample;
d. recording one or more images of the hydrogel.

Further provided herein is a kit for detecting a biological particle in a sample, said kit comprising:
a. polymer hydrogel reagents; and
b. nucleic acid amplification reagents, such as isothermal amplification reagents, the reagents being capable of generating a reaction product in the vicinity of particles having a specific target.

Also provided herein is a system for detecting a biological particle in a sample, said system comprising:
a. a holder for a cassette with a sample compartment,
b. imaging means capable of forming an image of the sample in the sample compartment on image acquisition means,
c. image processing means,
d. at least one illumination source capable of illuminating the sample in the sample compartment; and
e. thermostatically controlled heating means capable of heating and optionally cooling the sample in the sample compartment.

### DESCRIPTION OF DRAWINGS

FIG. 1. DNA vector, EEV604A-2, quantified by gLAMP in UV-cured hydrogel. PEGDA hydrogel comprising LAMP reagents and EEV604A-2 vector was cured with UV light and incubated for 1 hour at 65° C. The hydrogel was imaged at 4× in Xcyto® 10. Left, Data show a serial dilution of EEV604A-2. Right, fluorescence imaging of the amplicons emerging from the LAMP reaction in the PEGDA hydrogel. The experiment was performed in triplicates. Error bars show standard deviation.

FIG. 2. Enumeration of the RNA virus MS2 by gLAMP in UV-curedhydro. PEGDA hydro comprising LAMP reagents and MS2 bacteriophage was cured with UV light and incubated for 1 hour at 65° C. The hydro was imaged at 4× in Xcyto®10. Left, comparison of gLAMP-based enumeration of MS2 and PFU count. Right, fluorescence imaging of the amplicons emerging from the LAMP reaction in the PEGDA hydro. The gLAMP experiment was performed in triplicates. Error bars show standard deviation.

FIG. 3. Fluorescence imaging of the amplicons emerging from the LAMP reaction in the PEGDA hydro. Vector DNA EEV604A-2 quantified by gLAMP in UV-cured hydro. PEGDA hydro comprising LAMP reagents and EEV604A-2 vector was cured with UV light and incubated for 0, 30 and 60 minutes at 65° C. The hydro was imaged at 4× in Xcyto® 10. The circle shows an amplicon developing satellite amplicons. The dashed circle shows a false positive.

FIG. 4. Instrument workflow. The proposed instrument should perform two or more of the listed steps.

FIG. 5. DNA vector, EEV604A-2, quantified in fixed cells by gLAMP in UV-cured hydro. U2OS cells haboring EEV604A-2 were either MeOH fixed or NBF fixed and permeabilized with Triton X-100. PEGDA hydro comprising LAMP reagents was cured with UV light and incubated for 60 minutes at 65° C. The hydro was imaged at 4× in Xcyto® 10 using bright field (upper row) and fluorescence (lower row) imaging. Panel A shows images of MeOH fixed cells. Panel B shows images of NBF fixed cells.

FIG. 6. DNA vector, EEV604A-2, quantified in live cells by gLAMP in UV-cured hydro. U2OS cells haboring EEV604A-2 were washed in PBS. PEGDA hydro comprising LAMP reagents was cured with UV light and incubated for 60 minutes at 65° C. The hydro was imaged at 4× in Xcyto® 10 using bright field (upper row) and fluorescence (lower row) imaging.

FIG. 7. MAETAC reduces the formation of satellite amplicons. PEGDA hydro comprising LAMP reagents and EEV604A-2 vector with/without 2 mM MAETAC was cured with UV light and incubated for 60 minutes at 65° C. The hydro was imaged at 4× in Xcyto® 10. Figure shows fluorescence imaging of the amplicons emerging from the LAMP reaction in the PEGDA hydro without addition of MAETAC (left) and with addition of MAETAC (right).

FIG. 8. Digital gLAMP. Reaction mix comprising LAMP reagents, EEV604A-2 vector and PEGDA hydro components was UV cured through a photolithographic mask to create reaction wells, and incubated for 60 minutes at 65° C. The reaction wells were imaged at 4× in Xcyto® 10. Figure shows from left to right, UV bright field channel, fluorescence channel and a merged image.

FIG. 9. gLAMP detection of nucleic acids immobilized on beads. Reaction mix comprising LAMP reagents, PEGDA hydro components and target DNA bound to Dynabeads™ M-280 Streptavidin, was UV cured and incubated for 60 minutes at 65° C. The slide was imaged at 4× in Xcyto® 10. Figure shows from left to right, UV bright field channel, fluorescence channel and a merged image (all 200% zoom). Arrows indicate single beads with (a+b) or without LAMP signal (c).

FIG. 10. gLAMP detection of DNA with single nucleotide resolution. Reaction mix comprising LAMP reagents, sequence specific cleavage probes, Endonuclease IV and PEGDA hydro components was loaded onto a Xcyto 6-Chamber slide, hardened, and incubated for 60 minutes at 65° C. The slide was imaged at 4× in Xcyto® 10. Figure shows from left to right, FAM channel with signal from LEC-probe 1 and Cy5 channel with signal from LEC-probe 2 (all 200% zoom). From top to bottom either tGFP wt, tGFP mut or a mix of both templates was used. Arrows indicate exemplary amplicons in which primarily LEC-probe 1 (b) or LEC-probe 2 (a) was cleaved.

FIG. 11. DNA vector, EEV604A-2, visualized by gMDA in UV-cured hydro. PEGDA hydro comprising MDA reagents with EEV604A-2 vector and random primers was cured with UV light and incubated for 16 hours at 30° C. The hydro was imaged at 4× in Xcyto® 10. Left image, show gMDA amplicons in the fluorescent channel (400% zoom). Right image, show UV bright field channel (400% zoom).

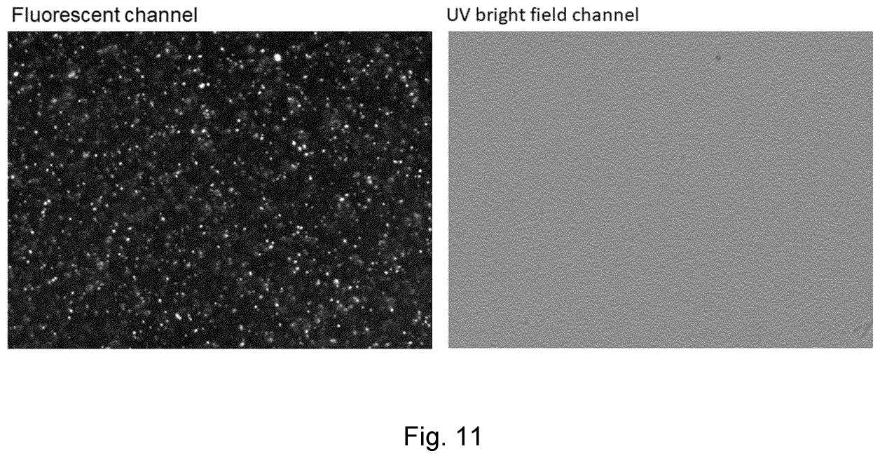

### DETAILED DESCRIPTION

The invention presented herein provides methods, kits and systems for detection and/or analysis of biological particles in a hydrogel, wherein curing of the hydrogel is inducible. In particular, the invention provides methods for detecting biological particles in a hydrogel using nucleic acid amplification reagents, such as isothermal amplification. This approach is advantageous compared to conventional isothermal reactions ran in single tubes, as the information gained from the methods of the invention is quantitative rather than qualitative. The amplification product remains in the vicinity of the biological particle and can thus be imaged using conventional cytometers and microscopes. This enables imaging of individual particles and quantitative assessment of reaction product for each particle. Thus, the methods developed by the inventors enable quantification of the amount, such as the concentration, of specific biological particles in a sample. Further, the hydrogel curing technique applied by the inventors is fast and inducible, which is advantageous as it minimizes the processing time as well as the impact on the biological particles before testing.

A ‘biological particle’ as used according to the present invention is a particle which comprises biological material, such as nucleic acids or proteins. In some embodiments, a biological particle is a cell, a virus, a viral particle, a bacteriophage, an exosome, a plasmid, RNA, an extrachromosomal DNA (ecDNA), an extrachromosomal circular DNA (eccDNA). A biological particle may also be a bead or some other type of solid to which a nucleic acid or protein is bound directly or indirectly. Examples of cells include a bacterium, a protozoan, a yeast, a fungus, a plant cell, an insect cell, and a mammalian cell. Thus, in some embodiments, the biological particle is a biological cell, a viral particle, a bacteriophage, a bead, an exosome, a plasmid, RNA, an extrachromosomal DNA (ecDNA), or an extrachromosomal circular DNA (eccDNA).

Thus, in one embodiment of the invention, the method for detecting or analyzing a biological particle in a sample comprises the steps of
a. mixing the sample with polymer hydrogel reagents and nucleic acid amplification reagents, such as isothermal amplification reagents, the isothermal amplification reagents being capable of generating a reaction product in the vicinity of particles having a specific target;
b. inducing curing of the mixture to a hydrogel;
c. incubating the hydrogel, whereby one or more reaction products are produced as a result of the amplification reaction;
d. recording one or more images of the hydrogel.

In some embodiments, steps b-d can be conducted in any order, preferably, step b is conducted before step c.

Thus, in another embodiment of the present invention, the method for detecting or analyzing a biological particle in a sample comprises the steps of
a. mixing the sample with polymer hydrogel reagents and nucleic acid amplification reagents, such as isothermal amplification reagents, the isothermal amplification reagents being capable of generating a reaction product in the vicinity of particles having a specific target;
b. incubating the mixture, whereby one or more reaction products are produced as a result of the amplification reaction;
c. inducing curing of the mixture to a hydrogel;
d. recording one or more images of the hydrogel.

In some embodiments, the recording of one or more images of the mixture or hydrogel is conducted before and/or after incubating the hydrogel or mixture.

In yet another embodiment of the present invention it is provided a method of detecting or analyzing a biological particle in a sample, said method comprising the steps of:
a. mixing the sample with curable polymer hydrogel reagents, the curable polymer hydrogel reagents comprising a photoinitiator;
b. illuminating the sample with a light pattern to induce gelling in illuminated parts thereby creating a hydrogel with a number of substantially liquid compartments separated by substantially gelled parts;
c. detecting or analyzing particles in the substantially liquid compartments.

In some embodiments, the reagents such as the polymer hydrogel reagents and/or the nucleic acid amplification reagents, such as isothermal amplification reagents comprise a primer set and optionally an aptamer, a molecular beacon aptamer, an antibody, a probe, or a receptor ligand with a nucleotide tag.

In some embodiments, a cellular stain is mixed with the reagents prior to curing the hydrogel.

In some embodiments, the biological particle is an extrachromosomal DNA (ecDNA) and/or an extrachromosomal circular DNA (eccDNA).

Thus, in some embodiments of the present invention, the method for detecting or analyzing ecDNA and/or eccDNA in a sample comprises the steps of
a. mixing nucleic acid amplification reagents, such as isothermal amplification reagents, curable polymer hydrogel reagents, and sample comprising ecDNA and/or eccDNA, the amplification reagents being capable of generating a reaction product in the vicinity of the ecDNA and/or eccDNA;
b. curing the mixture to a hydrogel;
c. incubating the mixture or hydrogel, whereby one or more amplicons are produced as a result of the amplification reaction amplifying whole or part of the ecDNA and/or eccDNA, if said ecDNA and/or eccDNA is present in the sample;
d. recording one or more images of the hydrogel.

In some embodiments, steps b-d can be conducted in any order, preferably, step b is conducted before step c.

In some embodiments, the ecDNA and/or eccDNA is or contains a marker of cancer, such as an oncogene, or a marker for a genetic disease or an autoimmune disease.

In some embodiments, the ecDNA and/or eccDNA is a plasmid.

In some embodiment, the biological particle is a virus and/or a viral particle.

Thus, in some embodiments of the present invention, the method for detecting or analyzing viral particles in a sample comprises the steps of:
a. mixing nucleic acid amplification reagents, such as isothermal amplification reagents, curable polymer hydrogel reagents, and sample comprising viral particles, the amplification reagents being capable of generating a reaction product in the vicinity of the viral particles;
b. curing the mixture to a hydrogel;
c. incubating the mixture or hydrogel, whereby one or more amplicons are produced as a result of the amplification reaction amplifying whole or part of the nucleic acids present in the viral particle, of said viral particle is present in the sample;
d. recording one or more images of the hydrogel.

In some embodiments, steps b-d can be conducted in any order, preferably, step b is conducted before step c.

Patterned Hydrogel

In many cases in the analysis of biological particles, such as cells, it is advantageous to perform the analysis on a single cell level. To enable this, the inventors have developed a method for dividing a liquid sample comprising biological particles into compartments. Depending on the concentration of biological particles the compartments can be designed to contain single particles, such as substantially only one biological particle or few particles, such as 2 or 3 particles. The method may be performed by illuminating a suspension of biological particles in a liquid sample comprising hydrogel reagents and a photoinitiator. By illuminating the sample with patterned light, gelled walls are generated to allow separation of biological particles into substantially liquid compartments.

The hydrogel walls prevent biological particles from floating from one compartment to another while maintaining the advantage of having biological particles suspended in liquid medium. By controlling the pattern and dilution of the suspension, one can control the number of biological particles in each compartment thus enabling single cell analysis.

Another preferred feature of having biological particles, and thus also any reagent, suspended in a liquid as opposed of being embedded in solid hydrogel, is that generally the rate of movements of reagents is substantially higher than the rate of movement in a solid hydrogel environment. This preferably increases the rate of the reaction, thus increasing the rate of product formation, which preferably can substantially reduce the time needed for any reaction. In the case when the product is soluble, this will similarly disperse the product in the liquid segment, which means that generally a substantially optimal size of the liquid section is preferred, the size being determined by factors such as reagent concentration, rate of reaction and time of reaction.

In one embodiment of the present invention, the method for detecting or analyzing a biological particle in a sample comprises the steps of
a. mixing the sample with curable polymer hydrogel reagents, the curable polymer hydrogel reagents comprising a photoinitiator;
b. illuminating the sample with a light pattern to induce gelling in illuminated parts thereby creating a hydrogel with a number of substantially liquid compartments separated by substantially gelled parts;
c. detecting or analyzing particles in the substantially liquid compartments.

In another embodiment of the present invention, the method of creating compartments in a sample with biological particles comprises the steps of:
a. mixing the sample with curable polymer hydrogel reagents, the curable polymer hydrogel reagents comprising a photoinitiator;
b. illuminating the sample with a light pattern to induce gelling in illuminated parts thereby creating a hydrogel with a number of substantially liquid compartments separated by substantially gelled parts.

In some embodiments, a pattern is made in the hydrogel, such as a pattern which generates multiple compartments.

In some embodiments, the compartments such as the liquid compartments are placed in rows in the hydrogel.

In some embodiments, labelling reagents are provided together with the hydrogel reagents, the labelling reagents being capable of generating a reaction product in the vicinity of particles having a specific target. The labelling reagents may be nucleic acid amplification reagents, such as isothermal amplification reagents.

In some embodiments, the labelling reagents comprise:
a. a primer set and optionally an aptamer, a molecular beacon aptamer, an antibody, a probe, or a receptor ligand with a nucleotide tag;
b. enzyme assay reagents, such as enzyme substrate, enzyme and/or enzyme co-factors; and/or
c. a protein linked to a nucleic acid tag, such as a DNA tag, and/or an antibody.

In some embodiments, the method further comprises the step of incubating the hydrogel to allow formation of the reaction products. In some embodiments, one or more reaction products are produced as a result of a reaction between the labelling reagents and a target, wherein the biological particle comprises and/and consists of said target.

In some embodiment, each substantially liquid compartment comprise a discrete number of biological particles, such as 0, 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 particles, preferably 0, 1, 2, 3, 4, 5, 6, 7, 8, 9 or 10 particles, more preferably 0, 1, 2 or 3 particles.

In some embodiments, the pattern is created using UV photolithography, wherein a collimated light source illuminates the sample through a photolithographic mask. In some embodiments, the pattern is created by inserting a mask in the light path between the light source and the sample, or by illuminating the sample with a light source that forms a light spot, which is projected in a predetermined pattern across at least a part of the sample. In some embodiments, the pattern is created by illuminating the sample with a laser.

The illumination may be carried out for a limited amount of time. In some embodiments, the duration of the step of illuminating the sample is less than 15 minutes, preferably less than 14 min, preferably less than 13 min, preferably less than 12 min, preferably less than 11 min, preferably less than 10 min, preferably less than 9 min, preferably less than 8 min preferably less than 7 min, preferably less than 6 min, preferably less than 5 min, preferably less than 4 min, preferably less than 3 min, preferably less than 2 min, preferably less than 1 min, preferably less than 30 seconds, preferably less than 15 seconds.

In some embodiments, the mixture is illuminated at a wavelength between 100 nm-1,200 nm, for example 100 nm-1,000 nm, such as 150 nm-850 nm, for example 200 nm-800 nm, such as 300 nm-750 nm, for example 350 nm-700 nm, such as 350 nm-600 nm, for example 375 nm-500 nm, such as 390 nm-400 nm. In other embodiments, the wavelength is between 100 nm-1200 nm, such as 150 nm, such as 200 nm, such as 250 nm, such as 270 nm, such as 300 nm, such as 350 nm, such as 365 nm, such as 375 nm, such as 380 nm, such as 385 nm, such as 390 nm, such as 395 nm, such as 400 nm, such as 444 nm, such as 450 nm, such as 500 nm, such as 524 nm, such as 550 nm, such as 600 nm, such as 650 nm, such as 700 nm, such as 750 nm, such as 800 nm, such as 850 nm, such as 900 nm, such as 950 nm.

In some embodiments, the mixture is illuminated at an intensity of 0.001 J/cm²-500 J/cm², such as 0.01 to 400 J/cm², for example 0.1 to 300 J/cm², such as 1 to 100 J/cm² for example 10-50 J/cm². In other embodiments, the intensity is between 0.001 J/cm²-500 J/cm², such as 0.01 J/cm², such as 0.1 J/cm², such as 0.2 J/cm², such as 0.25 J/cm² such as 0.3 J/cm², such as 0.5 J/cm², such as 0.75 J/cm², such as 0.1 J/cm², such as 0.5 J/cm², such as 1 J/cm², such as 5 J/cm², such as 10 J/cm², such as 15 J/cm², such as 20 J/cm², such as 25 J/cm², such as 30 J/cm², such as 50 J/cm², such as 75 J/cm² such as 100 J/cm², such as 150 J/cm², such as 200 J/cm², such as 250 J/cm², such as 300 J/cm², such as 350 J/cm², such as 400 J/cm², such as 450 J/cm².

In some embodiments, the area of the compartments such as the substantially liquid compartments is range 50-40,000 μm², for example 100-35.000 μm², such as 200-32,000 μm², for example 300-33,000 μm², such as 400-32,500 μm², for example 450-32,000 μm², such as 475-31,500 μm², for example 490-31,415 μm². In other embodiments, the area is between 50-5000μ², such as 100μ², such as 200μ², such as 300μ², such as 400μ², such as 500μ², such as 600μ², such as 700μ², such as 800μ², such as 900μ², such as 1000μ², such as 2000μ², such as 3000μ², such as 4000μ².

In some embodiments, the thickness of the substantially gelled wall part of the compartments is between 1-5,000 μm, such as 10-2,500 μm, for example 20-1,000 μm, such as 30-500 μm, for example 40-250 μm, such as 45-250 μm, such as 50-200 μm, such as 75-150 μm. In other embodiments, the thickness is between 1-5000 μm, such as 2 μm, such as 5 μm, such as 10 μm, such as 20 μm, such as 30 μm, such as 40 μm, such as 50 μm, such as 75 μm, such as 100 μm, such as 200 μm, such as 400 μm, such as 600 μm, such as 800 μm. Often, a gelled wall part of small thickness is preferred under conditions such as when the gel has low permeability of either reagents or products. Also, often a gelled wall part of large thickness is preferred when the gel has high permeability. In several embodiments the thickness of the wall part of the compartment is determined by a desired probability of a particle being placed in a liquid compartment rather than in the gelled part of the hydrogel.

In some embodiments, the height of the substantially liquid compartments is between 10-10,000 μm, for example 50-1,000 μm, such as 50-500 μm, for example 75-200 μm, such as 90-150 μm. In other embodiments, the height is between 10-10000 μm, such as 20 μm, such as 50 μm, such as 100 μm, such as 200 μm, such as 300 μm, such as 400 μm, such as 500 μm, such as 750 μm, such as 1000 μm, such as 2000 μm, such as 4000 μm, such as 6000 μm, such as 8000 μm.

In some embodiments, the volume of the substantially liquid compartments is between 100,000 μm³-40,000,000 μm³, for example 250,000 μm³-35,000,000 μm³, such as 400,000 μm³-32,500,000 μm³, for example 450,000 μm³-32,000,000 μm³, such as 490,000-31,415,000 μm³. In other embodiments, the volume is between 500 μm³-0.5 mm³, such as 750 μm³, such as 1000 μm³, such as 1500 μm³, such as 2500 μm³, such as 5000 μm³, such as 7500 μm³, such as 10000 μm³, such as 50000 μm³, such as 100000 μm³, such as 500000 μm³, such as 0.001 mm³, such as 0.0025 mm³, such as 0.005 mm³, such as 0.0075 mm³, such as 0.01 mm³, such as 0.025 mm³, such as 0.05 mm³, such as 0.075 mm³, such as 0.1 mm³, such as 0.25 mm³.

In some embodiments, the substantially gelled parts are solid. In some embodiments, the substantially gelled parts are cross-linked to limit diffusion of reagents such as labelling reagents.

In some embodiments, the pore size of the substantially gelled parts is between 1 nm-1,000 nm, such as 1 nm-900 nm, for example 1-700 nm, such as 5-500 nm, such as 10-400 nm, for example 15-300 nm, such as 16-200 nm, for example 17 nm-100 nm, such as 18-50 nm, such as 19-40 nm, such as 20-30 nm, such as 21-25 nm. In other embodiments, the pore size is between 0.1 nm-1000 nm, such as 0.5 nm, such as 1 nm, such as 5 nm, such as 10 nm, such as 15 nm, such as 16 nm, such as 17 nm, such as 18 nm, such as 19 nm, such as 20 nm, such as 21 nm, such as 22 nm, such as 23 nm, such as 24 nm, such as 25 nm, such as 30 nm, such as 40 nm, such as 50 nm, such as 100 nm, such as 200 nm, such as 300 nm, such as 400 nm, such as 500 nm, such as 700 nm, such as 900 nm. In one embodiment, the pore size of the substantially gelled parts is less than 0.1 nm.

In some embodiments, substantially no reaction product is formed in the substantially gelled parts. In other words, substantially no reaction product is formed outside of the substantially liquid compartments.

In some embodiments, compartments in a sample are substantially homogeneous in size and shape, while in other embodiments the size and shape are substantially different. In embodiments with substantially different compartment size or shape the analysis can be improved by allowing for conditions that are substantially suitable for varying properties of a sample, such as when the particle concentration of samples is expected to vary greatly, where small, and thus numerous compartments generally is suitable for samples with high particle concentration, while large and thus fewer compartments generally is suitable for samples with low particle concentration. Often it is preferred to have regions of the hydrogel with large and small compartments in separated parts of the sample, usually this is preferred when the approximate concentration of particles in the sample is not known beforehand.

In some embodiments, the general shape of the liquid compartment in the hydrogel is substantially square, while in other equally preferred embodiments the general shape is substantially rectangular, oval or circular. In several embodiments, different liquid compartments in the hydrogel have different general shapes. The preferred shape of liquid compartments in the hydrogel is often determined by properties such as the areal ratio between the liquid compartments and the gelled hydrogel, adjustment of the ratio between area and circumference of the liquid compartment.

In some embodiments, the general shape of the gelled part of the hydrogel has the shape of square, rectangular, oval or circular, such that gelled part of the hydrogel is surrounded, or substantially surrounded, by a liquid part of the sample. Further in some embodiments, such gelled part of the hydrogel contains liquid compartments.

In embodiments containing liquid compartments, there will inevitably be a probability function describing the likelihood of a particle being placed fully or in part, in a liquid compartment or in a gelled hydrogel segment. By knowing or estimating such probability function it is possible to make an estimate of the likelihood for the events that zero, 1, 2, 3, etc. particles would be founds in a liquid compartment, preferably such likelihood is related to the particle concentration of the sample being analyzed. It is often preferred to estimate the number of particles located in an individual liquid gel compartment by assessing property related to the total integrated signal generated from the gel compartment. Embodiments for the assessment of enumeration of particles in a sample use such probability function to estimate the number of particles in a sample based on the enumeration of particles in liquid compartments. In cases, especially when the concentration of particles in a sample is high, assessment of enumeration of particles in a sample is based on enumeration of particles in the gelled hydrogel part.

Often the property of, or related to, total integrated signal generated from the gel compartment appears to be digitized depending on the number of particles present in a segment, such that it is substantially possible to discriminate gel segments containing zero, 1, 2, 3 or even more particles, thus making it possible to directly count the number of particles present in a gel compartment. In other, equally preferred embodiments, the total integrated signal has the property that it is generally increasing with increasing numbers of particles in a liquid compartment, such as is generally the case when liquid compartments contain a high number of particles, such as 4 or more particles, in which case it is possible to estimate the number of particles in a liquid gel compartment rather than directly counting them. In embodiments where the analysis of particles contains the task of enumerating the number of particles in a sample, it is often preferred to combine the methods of digital assessment and intensity assessment.

In one embodiment, the patterned gel, i.e. the hydrogel with a number of substantially liquid compartments separated by substantially gelled parts, is used to isolate cells of interest. Cells of interest may for example be cells harboring a certain nucleic acid, or expressing high amounts of a certain protein, such as a nucleic acid or protein which can be detected using the methods described herein in the section “Target in biological particles”.

Thus, in one embodiment, the cells are partitioned in the substantially liquid compartments and analyzed as disclosed herein in the section “Detection and analysis”; cells with certain properties, such as cells comprising a certain nucleic acid or expressing high amounts of a certain protein, are isolated from the substantially liquid compartments. In one embodiment, said cells are isolated using robotic platforms for high-throughput screening.

The method presented herein may comprise additional steps, such as means for isolating a biological particle comprising a target.

In some embodiments, the biological particle is a cell, and the method further comprises isolating cells comprising the target from the substantially liquid compartments. In some embodiments, said cells are selected from the group consisting of bacteria, yeast, protozoa, fungus, plant cells, mammalian cells and insect cells, and the target is selected from the group consisting of a nucleic acid, an exosome, a plasmid, an RNA molecule, an extrachromosomal DNA (ecDNA), an extrachromosomal circular DNA (eccDNA), an aptamer, a nucleic acid tag linked to an aptamer, a molecular beacon, a nucleic acid tag linked to an antibody or a receptor ligand, an antibody, a protein and an enzyme.

The cells comprising the target may be identified and isolated using various means. In some embodiments, said cells are identified using a robotics platform. In some embodiments, said cells are isolated using a robotics platform.

Hydrogel

Hydrogels comprise a network of crosslinked polymers and/or monomers. Such crosslinks which bond the polymers or monomers may for example be physical crosslinks, which may comprise hydrogen bonds, hydrophobic interactions and/or chain entanglements, or chemical crosslinks, which may comprise covalent bonds. By modifying the concentration of polymer/monomer on the hydrogel, the mechanical properties of the hydrogel can be modified.

The different classes of hydrogels include homopolymeric hydrogels, copolymeric hydrogels, and multipolymer interpenetrating polymeric hydrogels. These classes comprise hydrogels with a polymer network derived from a single species of monomer or polymer; hydrogels comprised of two or more different monomer or polymer species; and hydrogels made of two independent cross-linked synthetic and/or natural polymer components contained in a network form, respectively (Ahmed et al., 2013).

Hydrogels may be prepared from diverse chemicals which provide hydrogels with different properties. The chemicals may for example be synthetic or natural polymers and/or monomers of various concentration and molecular weight, and may be used to generate hydrogels with a specific pore size. Thus, in some embodiments, the polymer hydrogel is prepared from chemicals selected from the group consisting of acrylics, methacrylics, acrylamides styrenics, norbonenes and thiols.

In some embodiments, the polymer hydrogel reagents comprise a polymer/monomer selected from the group consisting of polyacrylamide (PA), polyethylene glycol (PEG), polyethylene glycol diacrylate (PEGDA), dimethacrylated PEG (PEGDM), poly(ethylene glycol)-block-polylactide methyl ether (PEG-b-PLA), polyethylene glycol thiols (PEG-SH), polyethylene glycols norbornene-terminated (PEG-norbornene), poly-D,L-lactic acid/polyethylene glycol/poly-D,L-lactic acid (PDLLA-PEG), poly(vinyl alcohol) (PVA), chondroitin sulfate, chitosan, heparin, dextran, agarose, alginate, starch, pectin, polyvinylamine, polyphosphazene, poly(L-glutamic-acid), poly(N,N-dimethylacrylamide-co-furfuryl methacrylate), carboxymethylcellulose, poly(N-isopropylacrylamide), poly(aspartic acid), gellan gum, copoly(acrylamide), hydroxy propylmethylcellulose, collagen-inspired undecapeptide, collagen, fibrin, gelatin, silk fibroin, silk-MA (glycidyl-methacrylate-modified silk fibroin), hyaluronic acid, methacrylated hyaluronic acid, methacrylated gelatin, methacrylated alginate, methacrylated collagen, methacrylated peptide, peptide and DNA.

In some embodiments, the polymer hydrogel reagents comprise polyethylene glycol diacrylate (PEGDA) with a molecular weight between 0.5-100 K, such as 1-80 K, for example 1.5-60 K, such as 2-40 K, for example 2.5-35 K, such as 3-30 K, for example 3-25 K, such as 3-20 K, for example 3-10 K, such as 3-7 K, for example 3-5 K, such as 3.3-4 K, such as 3.4 K, such as 5 K, such as 10 K, such as 15 K, such as 20 K, such as 25 K.

In some embodiments, the concentration of monomer/polymer in the mixture is between 0.1%-40% (w/v), such as 1-25, for example 2-10, such as 5-10. In other embodiment, the concentration is 0.1%-40% (w/v), such as 0.5% (w/v), such as 0.75% (w/v), such as 1% (w/v), such as 2% (w/v), such as 3% (w/v), such as 4% (w/v), such as 5% (w/v), such as 6% (w/v), such as 7% (w/v), such as 8% (w/v), such as 9% (w/v), such as 10% (w/v), such as 11% (w/v), such as 13% (w/v), such as 15% (w/v), such as 17% (w/v), such as 19% (w/v) such as 20% (w/v), such as 25% (w/v), such as 30% (w/v), such as 35% (w/v).

In some embodiments, the pore size of the hydrogel or the substantially gelled parts is between 1 nm-1.000 nm, such as 1 nm-900 nm, for example 1-700 nm, such as 5-500 nm, such as 10-400 nm, for example 15-300 nm, such as 16-200 nm, for example 17 nm-100 nm, such as 18-50 nm, such as 19-40 nm, such as 20-30 nm, such as 21-25 nm. In other embodiments, the pore size is 0.1 nm-1000 nm, such as 0.5 nm, such as 1 nm, such as 5 nm, such as 10 nm, such as 15 nm, such as 16 nm, such as 17 nm, such as 18 nm, such as 19 nm, such as 20 nm, such as 21 nm, such as 22 nm, such as 23 nm, such as 24 nm, such as 25 nm, such as 30 nm, such as 40 nm, such as 50 nm, such as 100 nm, such as 200 nm, such as 300 nm, such as 400 nm, such as 500 nm, such as 700 nm, such as 900 nm.

In some embodiments, the pore size of the hydrogel or the substantially gelled parts is less than 0.1 nm.

The hydrogel may be homogenous or heterogeneous. In other words, the hydrogel may be homogenous or heterogeneous in terms of density, pore size, degree of solidity and height of the substantially liquid compartments. Thus, the hydrogel may or may not comprise two or more areas which are more or less solidified and dense, and/or areas which have different properties in terms of pore size and compartment height.

In some embodiments, the hydrogel is heterogeneous.

In some embodiments, the hydrogel contains two or more areas with different properties, such as areas with different pore size, density, degree of solidity and/or compartment height.

In some embodiments, the hydrogel contains two or more areas with different properties, such as areas of high degree of solidity and areas of substantially no solidity.

Agents may be added to the hydrogel in order to change its properties. Such agents may for example change the charge of the hydrogel. In other words, agents may be added to a hydrogel in order to make the hydrogel positively or negatively charged.

In some embodiments, an agent is added to the mixture in order to make the hydrogel positively charged.

In some embodiments, the agent is a positively charged polymer, such as poly-lysine and/or polyethylenimine.

In some embodiments, the agent is a positively charged monomer, such as 2-(methacryloyloxy)ethyl-trimethylammonium chloride (MAETAC), butyl methacrylate or [2-(acryloyloxy)ethyl]trimethylammonium chloride (AETAC).

In some embodiments, an agent is added to the mixture in order to make the hydrogel negatively charged.

In some embodiments, the agent is a negatively charged polymer, such as DNA, polystyrene sulfonate and/or polyacrylic acid.

In some embodiments, the agent is a negatively charged monomer, such as sodium 2-sulfoethyl methacrylate (SEMA).

In order to reduce diffusion of reaction product, one or more of the primers may be covalently bound to a hydrogel component, for example by using thiol- or acrydite modified primers.

In further embodiments, a protein or peptide is part of the hydrogel reagents.

Hydrogels may be crosslinked using various curing methods. Curing may for example be inducible, such as induced by changing temperature, or by applying a current, an electric field, a magnetic field, sound, radiation, or pressure. The change in temperature, application of a current, an electric field, a magnetic field, sound, radiation or pressure may be conducted for a certain amount of time in order to cure the hydrogel. Curing may also be carried out using a combination of different curing methods.

Thus, in some embodiments, curing of the hydrogel is induced by a change in temperature, application of a current, by radiation, or by pressure.

In some embodiments, curing is done in more than one step, such as in two steps.

In some embodiments, the step of curing of the mixture to a hydrogel is less than 30 minutes, preferably less than 10 minutes, preferably less than 5 minutes, preferably less than 1 min, preferably less than 30 seconds.

Hydrogels may for example be cured using electromagnetic radiation, such as ultraviolet (UV) light or visible light of a specific wavelength and/or at a specific amount of energy per delivery area. In order to cure a hydrogel using electromagnetic radiation, photoinitiators, which are compounds that cleave from the absorption of photons, are added to the mixture which will be cured. Once the photoinitiators are exposed to concentrated electromagnetic radiation, they will cleave and form free radicals which will begin a polymerization reaction that forms crosslinks between polymer strands. This curing method provides control over the degree of crosslinking of the hydrogel, as polymerization stops once the electromagnetic radiation source is removed. It is further advantageous over other curing methods, as it is a fast and efficient way of curing the hydrogel, minimizing the impact of biological particles and reactants comprised in the mixture which is cured.

Thus, in some embodiments the polymer hydrogel reagents comprise a photoinitiator.

In some embodiments, the photoinitiator is selected from the group consisting of lithium phenyl-2,4,6-trimethylbenzoylphosphinate (LAP), Irgacure 651, Irgacure 184, Irgacure 907, Irgacure 2959 (12959), diphenyl(2,4,6-trimethylbenzoyl)phosphine oxide (TPO), phenylbis (2,4,6-trimethylbenzoyl) phosphine oxide (BAPO), BAPO-OLi, BAPO-ONa, VA-086, eosin Y, riboflavin, camphorquinone, 1,4-bis(4-(N,N-bis(6-(N,N,N-trimethylammonium)hexyl)amino)-styryl)-2,5-dimethoxybenzene tetraiodide (WSPI), 2,5-bis-[4-(diethylamino)-benzylidene]-cyclopentanone (BDEA), 3,3′-((((1E,1′E)-(2-oxocyclopentane-1,3-diylidene)bis(methanylylidene))bis(4,1 phenylene))bis(methylazanediyl))dipropanoate (P2CK), and ruthenium.

In some embodiments, the photoinitiator is lithium phenyl-2,4,6-trimethylbenzoylphosphinate (LAP) Irgacure 2959 (12959).

In some embodiments, the concentration of photoinitiator in the mixture is between 0.0001%-5% w/w, such as 0.001 to 2.5%, for example 0.01 to 1%. In other embodiments it is 0.0001%-5% w/w, such as 0.0005% w/w, such as 0.001% w/w, such as 0.005% w/w, such as 0.01% w/w, such as 0.05% w/w, such as 0.1% w/w, such as 0.2% w/w, such as 0.3% w/w, such as 0.4% w/w, such as 0.5% w/w, such as 0.6% w/w, such as 0.07% w/w, such as 0.08% w/w, such as 0.9% w/w, such as 1% w/w, such as 1.5% w/w, such as 2% w/w, such as 2.5% w/w, such as 3% w/w, such as 3.5% w/w, such as 4% w/w, such as 4.5% w/w.

In some embodiments, the mixture is cured to a hydrogel at a wavelength 100 nm-1,200 nm, for example 100 nm-1,000 nm, such as 150 nm-850 nm, for example 200 nm-800 nm, such as 300 nm-750 nm, for example 350 nm-700 nm, such as 350 nm-600 nm, for example 375 nm-500 nm, such as 390 nm-400 nm. In other embodiments it is cured to a hydrogel at a wavelength between 100 nm-1200 nm, such as 150 nm, such as 200 nm, such as 250 nm, such as 270 nm, such as 300 nm, such as 350 nm, such as 365 nm, such as 375 nm, such as 380 nm, such as 385 nm, such as 390 nm, such as 395 nm, such as 400 nm, such as 444 nm, such as 450 nm, such as 500 nm, such as 524 nm, such as 550 nm, such as 600 nm, such as 650 nm, such as 700 nm, such as 750 nm, such as 800 nm, such as 850 nm, such as 900 nm, such as 950 nm.

In some embodiments, the mixture is cured to a hydrogel at an intensity of 0.001 J/cm²-500 J/cm², such as 0.01 to 400 J/cm², for example 0.1 to 300 J/cm², such as 1 to 100 J/cm², for example 10-50 J/cm². In other embodiments, the hydrogel is cured at an intensity of 0.001 J/cm²-500 J/cm², such as 0.01 to 400 J/cm², for example 0.1 to 300 J/cm², such as 1 to 100 J/cm², for example 10-50 J/cm². In other embodiments the intensity is 0.001 J/cm²-500 J/cm², such as 0.01 J/cm², such as 0.1 J/cm², such as 0.2 J/cm², such as 0.25 J/cm², such as 0.3 J/cm², such as 0.5 J/cm², such as 0.75 J/cm² such as 0.1 J/cm², such as 0.5 J/cm², such as 1 J/cm², such as 5 J/cm², such as 10 J/cm², such as 15 J/cm², such as 20 J/cm², such as 25 J/cm², such as 30 J/cm², such as 50 J/cm², such as 75 J/cm², such as 100 J/cm², such as 150 J/cm², such as 200 J/cm² such as 250 J/cm², such as 300 J/cm², such as 350 J/cm², such as 400 J/cm², such as 450 J/cm².

Hydrogels may also be cured using electromagnetic radiation and/or other methods, such as a current or a change in temperature. Hydrogels which may be cured by an increase in temperature include for example TissueSpec® ECM Hydrogel (Xylyx Bio), Geltrex® (Gibco), Matrigel® (Corning), CyGEL™ (Biostatus) and Cultrex® (R&D Systems). Further, current-based curing and/or other curing methods may be combined to modify the hydrogel properties, such as for example to decrease the hydrogel pore size.

In some embodiments of the invention, a current is used to cure the mixture to a hydrogel.

In some embodiments of the invention, the hydrogel is cured with a current after being cured by any other curing method. In other embodiments, the hydrogel is cured with a current after being cured by any of the curing methods described herein.

In some embodiments, curing the hydrogel with a current decreases the diffusion rates in the hydrogel.

In some embodiments, the polymer hydrogel reagents comprise an agent which enables the hydrogel to be cured by a change in temperature.

In some embodiments, the agent is selected from the group consisting of bovine serum albumin (BSA), laminin, type IV collagen, heparin sulfate proteoglycan, and/or entactin.

In some embodiments, the hydrogel is incubated at a temperature between 20° C. to 80° C., such as 25° C., such as 30° C., such as 35° C., such as 37° C., such as 40° C., such as 45° C., such as 50° C., such as 55° C., such as 60° C., such as 65° C., such as 70° C., such as 75° C.

In some embodiments, the hydrogel is cured by increasing the temperature between 20° C.-50° C., such as 25° C., such as 30° C., such as 35° C., such as 40° C., such as 45° C.

Target in Biological Particle

The present invention provides methods for detecting or analyzing biological particles in a sample. The biological particle may comprise any kind of biological particle and/or specific target. In some embodiments, the biological particle and/or the specific target is a nucleic acid, such as deoxyribonucleic acid (DNA) or ribonucleic acid (RNA), such as viral RNA or messenger RNA (mRNA). Nucleic acids are biopolymers composed of nucleotides, and can be found in all lifeforms on Earth. Naturally occurring nucleic acids consist of the natural basepairs adenine (A), cytosine (C), thymine (T), guanine (G) and uracil (U); however, nucleic acids may also comprise unnatural basepairs. For example, unnatural, protein-encoding basepairs have been inserted in engineered bacterial genomes (Zhang et al., 2017).

Nucleic acids create and store information. The coding region, or coding sequence, of a nucleic acid sequence specifies the amino acid sequence of the protein it encodes. Thus, a change of a single base pair, i.e. a mutation, of the nucleic acid molecule may result in disease or disorder if said mutation affects the amino acid sequence which the nucleic acid sequence encodes.

Organisms and cells, including viruses, bacteria, yeast, plant cells and mammalian cells, may contain different types of nucleic acids, such as for example nucleic acids in the form of chromosomes, plasmids, mitochondrial DNA, small interfering RNA, microRNA, transfer RNA, ribosomal RNA and extrachromosomal circular DNA (eccDNA). eccDNA is circular DNA found in human, plant and animal cells in addition to chromosomal DNA. A subtype of eccDNA, ecDNA, are primarily found in organelles in eukaryotes. ecDNA may serve important biological functions, and has been associated with genomic instability and various diseases. It has for example been shown that oncogene amplification on ecDNA is a frequent event in cancer (Verhaak et al., 2019). ecDNA is considered to be a primary mechanism of gene amplification, resulting in many copies of driver oncogenes and very aggressive cancers.

Nucleic acids may be engineered and synthesized, and for example used as tags to label antibodies or receptor ligands. This may facilitate antibody and/or receptor ligand detection, and enable detection of said molecules using nucleic acid-based amplification techniques. Other types of engineered nucleic acids include molecular beacons and aptamers. Molecular beacons are hairpin-shaped nucleotide molecules with an internally quenched fluorophore whose fluorescence is restored when the molecular beacon binds to a target nucleic acid sequence. They can for example be used to report the presence of specific nucleic acids. Aptamers are oligonucleotide or peptide molecules that bind to a specific target molecule. Both DNA and RNA aptamers are able to robustly bind to various targets.

In some embodiments, the biological particle or the target is nucleic acid. In some embodiments, said nucleic acid is a nucleic acid in a virus, a viral particle, a bacteriophage, a bacterium, a protozoan, a yeast, a fungus, a plant cell, an insect cell, a mammalian cell, an exosome, a plasmid, an RNA molecule an extrachromosomal DNA (ecDNA), an extrachromosomal circular DNA (eccDNA), an aptamer, a nucleic acid tag linked to an aptamer, a molecular beacon, a nucleic acid tag linked to an antibody or receptor ligand, or residual nucleic acid in pharmaceutical products.

Examples of mammalian cells which can be analyzed by the method described herein are cell lines from the group of CHO (Chinese hamster ovary (CHO) cells); MCF-7 (a breast cancer cell line); U2OS (human osteosarcoma cells cultivated from bone tissue); Jurkat cells; HeLa (human cervical cancer cell line); HEK-293 (Human kidney cell line); COS-7 (African green monkey kidney cell line); MDCK (Dog kidney cell line); NIH-3T3 (Mouse embryonic cell line), Schneider S2 (Fruit fly cell line) cells, HepG2 (human hepatocytoma cell line), Hep2 (human laryngeal cell line), KB (human pharyngeal cell line), U87 (glioblastoma-astrocytoma cell line), Saos-2 (Osteosarcoma), YAR (EBV transformed B-cells), Vero (African green monkey kidney cell line), BHK-21 (Hamster kidney cell line) and Sf9 (Insect (Spodoptera frugiperda) kidney cell line). Thus, in some embodiments, the mammalian cell is from a non-human cell line. In some embodiments, the mammalian cell is from a human.

In some embodiments, the nucleic acid is a marker of a disease, such as an oncogene or a marker of a genetic disease or autoimmune disease. In some embodiments said marker is a medical marker of a disease.

Nucleic acids can be amplified using polymerase chain reaction (PCR), a technology which was invented in the 1980's and has since been well established in the art. Most PCR methods rely on thermal cycling, where reactants are exposed to repeated cycles of heating and cooling. In one embodiment, amplification is by PCR. For the purposes of this invention, reference to PCR on a nucleic acid sample includes reverse transcription-PCR (RT-PCR). Specifically, when the nucleic acid sample is one or more target RNAs, or a population of RNAs (e.g., total mRNA), RT-PCR will be carried out on the target RNAs. The PCR amplification reagents provided herein can therefore include reagents for reverse transcription. Reverse transcription and PCR may be carried out in the hydrogel, by providing an RT-PCR master mix, or reverse transcription may be carried out before inclusion of the sample into the hydrogel. One of ordinary skill in the art will readily know whether RT-PCR or PCR is necessary, based on the nucleic acid sample that is originally isolated. In some embodiments, PCR amplification reagents comprise reagents for PCR, qPCR, or RT-qPCR.

Another method for amplifying nucleic acids is through the use of isothermal nucleic acid amplification. In contrast to PCR, isothermal amplification is carried out at a constant temperature, and does not require thermal cycling. There are various methods and techniques to perform isothermal amplification, such as for example rolling circle amplification (RCA), loop mediated isothermal amplification (LAMP), and multiple displacement amplification (MDA). Thus, in some embodiments, the biological particle is a target nucleic acid and the reaction product is a nucleic acid amplified from said target nucleic acid. In some embodiments, the labelling reagents comprise reagents for isothermal amplification. In some embodiments, the isothermal amplification reagents comprise reagents for rolling circle amplification (RCA), loop mediated isothermal amplification (LAMP), multiple displacement amplification (MDA), nucleic acid sequence-based amplification (NASBA), helicase-dependent amplification (HDA), recombinase polymerase amplification (RPA), whole genome amplification (WGA), self-sustained sequence replication (3SR), transcription mediated amplification (TMA), signal-mediated amplification of RNA technology (SMART), ramification amplification (RMA), proximity ligation assay (PLA) and/or strand displacement amplification (SDA).

In some embodiments, the target is a nucleic acid and the reaction product is a nucleic acid amplified from said target nucleic acid.

Isothermal methods typically employ unique DNA polymerases for separating duplex DNA. For the amplification reaction to progress, dNTPs are typically also added to the reaction. In order to allow detection of RNA, the isothermal amplification ingredients may further comprise a reverse transcriptase which transcribes the RNA into DNA. Thus, in some embodiments, the isothermal amplification reagents comprise
a. a DNA polymerase, such as Phi29 or mutant versions thereof such as EquiPhi29 or QualiPhi; BST or mutant versions thereof such as large fragment, BST 2.0, BST 3.0 or IsoPol BST; BSU; BSM; IsoPol SD+; and/or Klenow; and
b. dNTPs; and optionally,
c. a reverse transcriptase.

Isothermal amplification can be carried out at various temperatures. Thus, in some embodiments the hydrogel and/or the mixture in which the isothermal amplification reaction takes place is incubated at a temperature between 20° C. to 80° C., such as 30° C., such as 35° C., such as 37° C., such as 40° C., such as 45° C., such as 50° C., such as 55° C., such as 60° C., such as 65° C., such as 70° C., such as 75° C.

Various additives may be added to the amplification reaction, such as to the isothermal reaction. Such additives may for example improve reaction progression and/or performance by reducing secondary structures of the nucleic acid to be amplified; neutralizing inhibitory components in the reaction; increase stringency of primer annealing; increase yield and specificity of the reaction; and/or reducing amplification of off-target DNA. Thus, in some embodiments, the reagents such as the isothermal amplification reagents and/or the labelling reagents further comprise dimethyl sulfoxide (DMSO), Propyl sulfoxide, Methyl sec-butyl sulfoxide, Tetramethylene Sulfoxide, Methyl sulfone, Sulfolane, single-stranded DNA binding protein (SSB), TaqSSB, T4 gene 32 protein (gp32), RecA, Tth RecA, nicking endoncleases (e.g. Nt.BsmAl, Nt.BstNBI, Nb.BsrDI), PEG4000, PEG400, Non-ionic detergents (such as, Tween-20, Triton X-100, NP-40), Glycerol, Propylene glycol, Ethylene glycol, Betaine, Trehalose, Formamide, Acetamide, 2-pyrrolidone, Tetramethyl ammonium chloride (TMAC), TMA oxalate, TMA hydrogen sulfate, TEA-CI, TPA-CI, TPA-acetate, TBA-CI, 7-deaza-2′-deoxyguanosine, Magnesium, Dithiothreitol (DTT), pyrophosphatase and/or bovine serum albumin (BSA).

The nucleic acid reaction product, i.e. the amplified nucleic acid, of the nucleic acid amplification, such as isothermal amplification may be visualized using detection dyes or agents which precipitate with reaction products and/or byproducts. Such dyes or agents may be added to the nucleic acid amplification, such as isothermal reaction in order to visualize the reaction product. Thus, in some embodiments, the nucleic acid amplification reagents, such as isothermal amplification reagents further comprise magnesium pyrophosphate, calcium pyrophosphate, hydroxynaphthol blue or calcein. In some embodiments, the magnesium or calcium pyrophosphate is deposited in the vicinity of said biological particle as a result of the isothermal amplification reaction.

Labelling the amplified nucleic acid reaction with a dye which improves and/or facilitates detection of said nucleic acid, and may enable detection of reaction product in real-time. Such dyes may for example bind to the reaction product, or may be incorporated during the isothermal amplification reaction. Thus, in some embodiments, the isothermal amplification reagents further comprise a dye to label the amplified nucleic acid. In some embodiments, the dye is linked to a primer or to deoxynucleotide triphosphates (dNTPs) which are incorporated during the isothermal amplification.

Dyes which may be used for labelling the amplified nucleic acid include intercalating dyes, which insert into DNA during amplification, and fluorescent probes, which are fluorescently labelled nucleotides that also may insert into DNA during amplification. Such probes absorb light of a specific wavelength and emit light of a different, typically longer, wavelength. They may for example be coupled to a primer or to dNTPs. Probes are often used together with a quencher. The quencher suppresses, i.e. quenches the emission, of the fluorescent probe in the presence or absence of target. In other words, the probe may be quenched unless it is incorporated into DNA.

In some embodiments, the dye is an intercalating dye or a fluorescent tagged probe.

In some embodiments, the intercalating dye intercalating dye is SYBR Green, SYBR Gold, Syto 9, Syto 80, SYBR 14, Syto 41, Syto 60, Syto 62, Syto 64, LAMP Dye, DAPI, Hoechst, ToPro3, Draq5, Draq7, RedDot1, RedDot2, Propidium iodide, Ethidium bromide and/or Evagreen.

In some embodiments, the fluorescent probe is FITC, FAM, PE, APC, Cy3, Cy5, Cy7, PerCP, AF488, AF647, AF555, CF488 CF555, CF647, any of the fluorescent probes from the Alexa fluorophore family, any of the fluorescent probes from the Biotium fluorophore family, any of the fluorescent probes from the Atto fluorophore family, such as Atto 488 and Atto 565, quantum dots, and/or a polymer dye from the Brilliant violet dye family.

In some embodiments, the multiple displacement amplification (MDA) or loop mediated isothermal amplification (LAMP) reagents comprise a primer-dye and a primer-quencher set. A “quencher” refers to a moiety that causes the detectable signal of a fluorescent moiety to decrease when the quencher moiety is sufficiently close to the fluorescent moiety. A “quencher” may quench by absorbing energy emitted from a fluorophore, or otherwise interfering with the ability of the fluorescent dye to emit light. A quencher can re-emit the energy absorbed from a fluorophore in a signal characteristic for that quencher, and thus a quencher can also act as a flourophore (a fluorescent quencher). This phenomenon is generally known as fluorescent resonance energy transfer (FRET). Alternatively, a quencher can dissipate the energy absorbed from a fluorophore as heat (a non-fluorescent quencher).

Amplification of the target such as nucleic acid can be performed in order to increase the amount of target, such as the concentration of the target. This is also known as pre-amplification. Pre-amplification may be used to increases the number or targets molecules prior to incorporation into the hydrogel. Suitable methods for pre-amplification include polymerase chain reaction (PCR), multiple displacement amplification (MDA), strand displacement amplification (SDA), recombinase polymerase amplification (RPA), multiple annealing and looping based amplification cycles (MALBAC), and primary template assisted amplification (PTA).

In some embodiments, the methods can be used for detection and/or differentiation of single nucleotide polymorphisms (SNPs) in a target nucleic acid, whereby at least one population of biological particles, such as at least two populations of biological particles can be detected and/or differentiated from each other. Example 9 demonstrates differentiation between two populations of biological particles differing only by a single nucleotide. The methods can be used for differentiate two, three, four, five, six, seven, ten or more populations using appropriate primers.

One population of biological particles may comprise at least a wild-type allele or a mutant allele; or the at least two populations of biological particles may comprise at least a wild-type and at least one mutant allele, or two different mutant alleles.

Reagents suitable for SNP detection include reagents such as reagents for loop-primer endonuclease cleavage—loop-mediated isothermal amplification (LEC-LAMP), PA-LAMP, RALA-LAMP or CHB-LAMP.

As stated above, the biological particle may comprise any kind of biological particle. In some embodiments, the target biological particle is a protein, such as an enzyme, and the method according to the invention comprises adding reagents or labelling reagents capable of binding said protein, the reagents or labelling reagents comprising a nucleic acid tag, such as a DNA tag and/or an antibody. In some embodiments, the reagent capable of binding said protein comprises an aptamer, a molecular beacon aptamer, an antibody, or a receptor ligand.

In some embodiments, the labelling reagents comprise a targeting moiety, such as an antibody, linked to an enzyme, wherein said targeting moiety is capable reacting with and/or binding the target and/or the biological particle.

In some embodiments, the biological particle and/or the specific target is a protein, and the reagent comprises a substrate and a reagent or labelling reagent capable of binding said protein, the reagent being linked to an enzyme capable of converting said substrate to the reaction product at a temperature optimum between 30° C. to 80° C., such as 35° C., such as 37° C., such as 40° C., such as 45° C., such as 50° C., such as 55° C., such as 60° C., such as 65° C., such as 70° C., such as 75° C.

In some embodiments, the agent further comprises compounds which enable the enzyme to convert the substrate into the reaction product, such as additional substrates and/or enzyme co-factors.

In some embodiments, the target is an enzyme and the labelling reagents comprise a reagent capable of reacting with said enzyme, the reagent comprising an enzyme substrate and/or enzyme co-factors.

In some embodiments, the enzyme is selected from the group of enzymes consisting of phosphatases such as alkaline phosphatase, β-galactosidase, β-glucuronidase, β-glucose-6-phosphate dehydrogenase, glucose oxidase, urease, luciferase, β-lactamase, β-amylase, and peroxidase, such as for example horseradish peroxidase.

The reaction product, such as the nucleic acid and/or any other reaction product, formed in the vicinity of the biological particle may be detected with various methods depending on the properties of said reaction product. In some embodiments, the reaction product is a product that causes reflection, refraction, diffraction, interaction, scattering or absorbance of light emitted from a light source onto the sample. In some embodiments, the reaction product is a fluorescent molecule, or a fluorescent molecule can bind to the reaction products. In some embodiments, the reaction product is coloured.

In some embodiments, the reaction product is unable to diffuse through the hydrogel. In some embodiments, the reaction product is unable to diffuse through the substantially gelled parts.

Sample

The sample to be analyzed may be any type of sample, such as a biological sample, comprising said biological particle. Thus, in some embodiments, the sample comprises viruses, viral particles, bacteriophages, bacteria, protozoa, yeast, fungi, plant cells, insect cells, mammalian cells, exosomes, proteins, enzymes, plasmids, DNA, RNA, mRNA, microRNA, ecDNA, eccDNA, and/or residual nucleic acid in pharmaceutical products. In some embodiments, the mammalian cells comprised in the sample are from a non-human cell line. In some embodiments, the mammalian cells comprised in the sample are human cells.

Further, the sample may be any type of sample which may comprise said biological particle. In some embodiments, the sample is a body fluid sample, a tissue sample, a fermentation sample, a liquid cultivation sample, a cell culture sample, a water sample, a beverage sample, a pharmaceutical sample, an environmental sample, a sewage sample, a diagnostic sample or a sample of a pharmaceutical product. In some embodiments, the body fluid sample is selected from the group consisting of samples from swabs, blood, plasma, serum, urine, bile, saliva, semen, cerebrospinal fluid and mucus. In some embodiments, the tissue sample is selected from the group consisting of tissue samples from liver, kidney, muscle, brain, lung, skin, thymus, spleen, gastrointestinal tract, pancreas and thyroid gland. In some embodiments, the sample is a sample from a human.

Biological staining may be used to stain samples prior to processing the sample according to the method presented herein. Biological stains, or dyes, may for example be used to stain tissues, whole organisms, cells and/or specific organelles. Several stains may be used for the same sample. In some embodiments, the stain is a cellular stain which is added to the mixture prior to curing the hydrogel. In some embodiments, a cellular stain is added to the mixture prior to curing the gel. In some embodiment, the cellular stain is a stain which stains the whole cell or part of the cell, such as the cell membrane, the cell wall, the cytoplasm, the nucleus, the mitochondria and/or nucleic acids.

The sample may be pre-treated with any suitable treatment, such as centrifugation, sedimentation, filtration, extraction, dilution, irradiation, agitation, addition of chemicals, chromatographic separation. Furthermore, pre-treatment may include lysing of the sample, and/or other methods. Thus, in some embodiments, a lysing agent is added to the sample or to the mixture prior to curing the hydrogel. In some embodiments, the sample is pre-treated using a method comprising one or more of the following steps:
a. incubating the sample at 98° C. for 5 minutes; and/or
b. mixing the sample with 96% ethanol; and/or
c. mixing the sample with 1-1000 mM sodium hydroxide; and/or
d. mixing the sample with detergent, such as NP40, tween20 or triton X100; and/or
e. mixing the sample with saponin; and/or
f. mixing the sample with 96% methanol.

In some embodiments, the method further comprises illuminating the hydrogel, whereby cells comprised in the hydrogel are lysed.

Sample Compartment

The sample as described above may be processed and loaded into a sample compartment, wherein the mixture may be cured. The sample compartment which may or may not be part of a cassette or a system. Thus, in some embodiments, the sample is loaded into a sample compartment, such as wherein the mixture is cured inside said sample compartment. In some embodiments, the sample compartment forms part of a cassette. The sample compartment may have any shape and comprise any type of material which is appropriate for conducting the method as presented herein. In some embodiments, the sample compartment is defined by two parallel sheets of transparent material, preferably plastic material or glass material with a give distance between them. In some embodiments, at least one wall of the sample compartment is transparent.

The sample is contained in the interior of the sample compartment, which normally has an average thickness of between 20 μm and 2000 μm, usually between 20 μm and 1000 μm and in many practical embodiments between 20 μm and 200 μm.

Normally, the sample compartment has dimensions, in a direction substantially parallel to a wall of an exposing window, in the range between 1 mm by 1 mm and 100 mm by 100 mm, such as 10 mm by 10 mm, but it will be understood that depending on the design, it may also be larger and, in some cases, smaller.

The part of the sample compartment allowing detection and analysis of the biological particles may be referred to as the exposing window, which can be as little as 0.01 mm² or more, preferably with an area of 0.1 mm² or more, more preferably with an area of 1 mm² or more, preferably with an area of 2 mm² or more, preferably with an area of 4 mm² or more, preferably with an area of 10 mm² or more, preferably with an area of 20 mm² or more, preferably with an area of 40 mm² or more, more preferably with an area of 100 mm² or more, preferably with an area of 200 mm² or more, preferably with an area of 400 mm² or more, preferably with an area of 1000 mm² or more, preferably with an area of 2000 mm² or more, preferably with an area of 4000 mm² or more, preferably with an area of 10000 mm² or more. Similarly, it is advantageous to extend the window of the sample compartment in a direction which is parallel to the plane of any window exposing signals from the sample to the exterior in order to extend the area of the exposing window and thus increase the volume of the sample which is exposed to the exterior.

The requirements of the wall of the sample compartment are in particular that the wall allows the detection and analysis of the biological particles without any significant limitations. In practice no upper limit is given for the wall thickness apart from what is defined by cost and design. The wall is preferably a substantially stable wall, which leads to a lower thickness limit for each material used. Preferably, the wall is from 0.1 mm to 2 mm, such as from 0.5 mm to 1.5 mm, more preferred from 0.75 mm to 1.25 mm.

The volume of the liquid sample from which electromagnetic radiation is exposed onto the array of detection elements may be in the range between 0.01 μL and 20 μL, such as between 0.05 μL and 5 μL. As the total volume of the sample being assessed, which is exposed onto the array of detection elements depends on several factors, including the sample thickness and area of the part of the sample compartment that is exposed onto the array of detection elements, it is possible to combine several features of the present invention in order to determine this volume. Often this results in a volume that is analysed in a single exposure that is between 0.05 μL and 1.0 μL.

The cassette which comprises the sample compartment may further comprise various parts, such as channels and/or valves, which for example may be used for transfer of the sample. In some embodiments, the cassette comprises channels for loading the sample and/or channels for flowing the sample within the cassette. In some embodiments, the cassette comprises a valve for regulating the flow of the sample. The cassette as well as the parts comprised in the cassette may be removable and/or disposable. In some embodiments, the cassette is removable and/or single-use and/or disposable.

The sample compartment and any other part of the cassette may comprise any type of reaction agents preloaded in said compartment or part. Some or all of the hydrogel reagents and nucleic acid amplification reagents may be pre-loaded into the cassette in dry or freeze-dried form. For use, one only needs to add a liquid sample with biological particles. The pre-loaded reagents will dissolve sometimes after gentle shaking and the cassette can be exposed to induction of gel formation and nucleic acid amplification.

In some embodiments, the photoinitiator described in the section “Hydrogel” or anywhere else herein is preloaded into the sample compartment or into a channel part of the cassette.

In alternative embodiments, the cassette may contain a preformed and re-dried hydrogel with nucleic acid amplification reagents. By adding the biological particles suspended in a water based buffer, the hydrogel is re-hydrated and the nucleic acid amplification reaction can be initiated.

Detection and Analysis

The method according to the present invention may comprise further steps, such as steps for detecting and analyzing the reaction product. In other words, the invention may comprise steps where the amount of nucleic acid and/or the number of biological particles is quantified. In some embodiments, the method further comprises counting the number of particles and the number of particles with reaction product in at least part of the sample. The reaction product may be detected on the hydrogel as spots. Thus, in some embodiments, the method further comprises quantifying the amount, such as the concentration, of target, by counting the number of spots with reaction product present in the hydrogel, and/or optionally counting the total number of particles in the hydrogel.

In some embodiments, the method further comprises quantifying the amount, such as the concentration, of target, by counting the amount of spots present in the hydrogel. In some embodiments, the number of spots is counted in an image using image analysis to identify spots.

In another embodiment, the amount, such as the concentration, of biological particles and/or target in the sample is quantified by counting the number of substantially liquid compartments with reaction product. In some embodiments, the number of substantially liquid compartments with reaction product are counted in an image using image analysis to identify the number of substantially liquid compartments with reaction product.

Detection and analysis of the reaction product can be done by analyzing the formation and appearance of spots or substantially liquid compartments with reaction product over time and/or at the end of the reaction process. Such detection and analysis may for example be done by recording one or more images of the hydrogel or mixture before and during incubation. Further, the background, i.e. the objects, spots or substantially liquid compartments with reaction product present before the reaction initiates, may be identified by recording one or more images of the hydrogel or mixture before incubation. Thus, in some embodiments, the method according to the present invention further comprises recording an image of the hydrogel or mixture before incubation and/or induction of hydrogel formation. In some embodiments, said image is used to determine the background and/or background properties of the hydrogel or mixture before incubation. In some embodiments, multiple images of the hydrogel or mixture are recorded while said hydrogel or mixture are being incubated, such as wherein said images are compared following image processing to determine the number of spots or substantially liquid compartments with reaction product. In some embodiments, the image processing determines the amount of reaction product in each substantially liquid compartment. In some embodiments, said amount is correlated to the discrete number of biological particles in each substantially liquid compartment. In some embodiments, multiple images are used to evaluate the formation of the reaction product over time.

In some embodiment, it is preferred that only the part of the sample which is to be imaged and analyzed is illuminated with a light pattern to induce gelling in illuminated parts. In other words, a hydrogel with a number of substantially liquid compartments separated by substantially gelled parts is only formed in the area of the sample where one or more images will be taken.

Images of the mixture and/or the hydrogel may be recorded using any type of recording instrument. The image can for instance be recorded by an array of detection elements, the array of detection elements comprising individual elements each of which is capable of imaging part of the hydrogel and/or mixture, the array as a whole being capable of imaging signals from substantially all of the hydrogel and/or mixture, or at least a well-defined part of the hydrogel and/or mixture. Thus, in some embodiments, the images are recorded using an array of detection devices. In some embodiments, the images are recorded using a two-dimensional array of detection devices.

In some embodiments, detection means comprises a detector being an array of detecting devices or detection elements, such as a charge coupled device (CCD), the CCD may be a full frame CCD, frame transfer CCD, interline transfer CCD, line scan CCD, an e.g. wavelength intensified CCD array, a CMOS, a video camera, photomultiplier tubes, photodiode, avalanche photodiode (APC), or a photon counting camera. Thus, in some embodiments, the images are recorded using a CCD, a CMOS, a video camera, photomultiplier tubes, photodiode, avalanche photodiode (APD) or a photon counting camera.

Said recording instrument may contain one or more optical and/or fluorescent channels, which may be used to detect the reaction product. The detection means may comprise any detectors capable of sensing or detecting the reaction product in the hydrogel and/or mixture, such as a fluorescence signal. Use of two or more channels is especially suitable if the reaction product is labelled with two or more dyes or fluorophores.

Confocal scanning optical microscopes offer a number of advantages over traditional optical microscopes. One main advantage of a confocal scanning microscope is that it provides optical sectioning of a sample because it attenuates light which is not in focus. Thus, only light which is in focus contributes to the final image. Thus, in some embodiments, the recording of images comprises the use of a confocal scanner, a microscope, a fluorescence microscope, an image cytometer, and/or an automated particle counter. In some embodiments, the recording of images comprises the use of one or more optical channels. In some embodiments, the recording of images comprises the use of one or more fluorescent channels.

When using bright field illumination, it is preferred that the incident light on the sample is collimated. In optical setup, it may be accepted that a perfectly collimated light is not always possible to obtain, and the collimated light from the bright field light source may deviate from collimated light with a deviation angle less than 10 degrees, more preferably less than 5 degrees.

In embodiments of the present invention, the wavelength from the bright field light source is between 200 nm and 700 nm. Preferably, the wavelength from the bright light source may be between 300 nm and 395 nm. Even more preferably, the wavelength from the bright field light source may be between 320 nm and 380 nm. Most preferably, the wavelength from the bright field light source may be between 350 nm and 380 nm.

The excitation light from the fluorescence light source may have a wavelength substantially different from the wavelength of light from the bright field light source. Preferably, the incidence angle of the excitation light may be between 10 and 80 degrees, preferably between 20 and 60 degrees, and more preferably between 30 and 50 degrees. Alternatively, the incidence angle of the excitation light may be 90 degrees. In another alternative embodiment of the present invention, the incidence angle is between 110 and 180 degrees, preferably between 120 and 160 degrees, and more preferably between 130 and 150 degrees.

In some embodiments of the present invention, the focusing means comprises a lens, whereas in another equally preferred embodiment of the present invention, the focusing means comprises a curved mirror.

In some embodiments of the present invention, the image cytometer further comprises a light source in addition to a bright field and a fluorescent light source, such as a third or fourth or fifth or sixth light source, preferably where the additional light sources are excitation light sources. The light source(s) may be a light emitting diode and/or a diode laser and/or a laser such as tunable solid-state light source(s) and/or a tunable light emitting diode. The tunable solid-state light source may be a tunable laser diode.

In some embodiments of the present invention, the light source(s) is/are optically connected to optical means configured for providing light with a substantially uniform intensity across the hydrogel region and/or across a region imaged by the array of detection elements. The optical means may comprise an array of micro lenses. Alternatively, the optical means may comprise an array of cylindrical micro lenses, preferably it may comprise two arrays of cylindrical micro lenses with substantially perpendicular orientation of the cylindrical lenses.

According to the present invention, the imaging light source(s) is/are configured for emitting light in duration less than 1 second, preferably for less than 0.1 second. Preferably, the light source(s) is/are configured for emitting light in duration between 0.0001 and 0.1000 second, preferably between than 0.0001 and 0.0500 second. However, in some situations such as when high sensitivity is required in fluorescence imaging, the light source(s) may be configured for emitting light in duration for more than a 1 second, such as for more than 2 seconds, such as for more than 3 seconds, such as for more than 4 seconds, such as for more than 5 seconds, such as for more than 6 seconds, such as for more than 7 seconds, such as for more than 8 seconds, such as for more than 9 seconds or such as for more than 10 seconds.

In one embodiment of the present invention, light from the excitation light source may substantially be eliminated from reaching the entrance of the image forming means by selectively removing rays of light from the beam of excitation light. The rays may be selectively removed by placing one or multiple obstructions in the beam of excitation light, preferably where the beam of excitation light is substantially collimated in the plane where the obstruction is placed.

In some embodiments of the present invention, the image forming means are configured for providing a depth of field that is more than 5 μm, such as between 10 μm and 150 μm. In this way, the hydrogel may be in focus in the depth of field such that the image forming means and/or the array of detection elements may not need to be moved in order to acquire a sharp image of the hydrogel, for example when the hydrogel has spots or substantially liquid compartments with reaction product positioned at different depths. However, in some embodiments of the present invention, the image forming means and/or the array of detection elements and/or the sample compartment may be configured for moving such that image forming means and/or the array of detection elements may be placed at an optimal position relative the hydrogel. One spot or substantially liquid compartment with reaction product, or a part of a spot or substantially liquid compartment with reaction product, may for example be in focus in one configuration, but when changing to another hydrogel or to a different part of the hydrogel, another spot or substantially liquid compartment with reaction product may then not be in focus, and it may thus be required to either move the hydrogel and/or the image forming means and/or the array of detection elements.

In preferred embodiments of the present invention, the image forming means is configured for transmitting light in the wavelength region of between 200 nm and 1000 nm, more preferably in the wavelength region of between 350 nm and 1000 nm, more preferably in the wavelength region of between 350 nm and 850 nm.

In some embodiments of the present invention, the image forming means comprises a microscope objective. The image forming means may be configured for providing a linear enlargement of the sample. Preferably, the linear enlargement is smaller than 20:1. The linear enlargement may also be in the range from 1:1 to 20:1, preferably in the range from 1:1 to 10:1, more preferably in the range from 1:1 to 4:1.

In some embodiments of the present invention the bright field light source is a light emitting diode (LED) and/or a diode laser and/or laser. Several of the properties of LED's and laser diodes offer substantial advantages in the design and operation of system according to the present invention, such as small physical size and high power efficiency. In many preferred embodiments of the present invention the wavelength of the light from the bright light source is less than 400 nm. It is often preferred that the wavelength of the light from the bright field light source is between 200 nm and 400 nm, such as between 300 nm and 395 nm. It has surprisingly been found that the use of light of short wavelength offers substantial improvement in the assessment of biological particles according to the present invention and light in wavelength bands such as 200 nm to 250 nm, 200 nm to 300 nm, 250 nm to 350 nm, and 320 nm and 380 nm are all preferred.

As stated above, the reaction product may be detected on the hydrogel as spots or substantially liquid compartments with reaction product. Sometimes the spots or substantially liquid compartments with reaction product being assessed may be sensitive to light, to a degree where it can alter the properties of the spots or substantially liquid compartments with reaction product, and one preferred method to reduce the effect of the light is to limit the length of time the sample is exposed to the light is to limit the time that the light source emits light onto the sample, preferably where the duration of illumination of light from the light source is less than 1 second, more preferably where it is less than 0.1 second. In other equally preferred embodiments illumination period is between 0.0001 and 0.1000 seconds, such as between 0.0001 and 0.0500 seconds. Expressed in energy, it is preferred that the hydrogel is illuminated with 200 nJ/mm2 or less, such as 100 nJ/mm2 or less, preferably 50 nJ/mm2 or less, such as 20 nJ/mm2 or less during exposure.

One method for the assessment of spots or substantially liquid compartments with reaction product in the hydrogel, according to the present invention, is based on recording an image of spatial light intensity information from the volume of sample where signal from individual spots or substantially liquid compartments with reaction product is attenuated light intensity signal relative to light intensity from the background. The attenuation can be brought about by one or several of the following, reflection, refraction, diffraction, interference, absorption, scattering. In these embodiments the light relating to a spot or substantially liquid compartments with reaction product is lower in intensity than the signal from the background and on the bases of this it is possible to process the image in a manner where the signal from individual spots or substantially liquid compartments with reaction product and signal from the background are distinct from each other, preferably where the signal from the spot or substantially liquid compartment with reaction product is substantially less than the signal from the background, preferable signal from the background in close spatial proximity to the spot or substantially liquid compartment with reaction product.

In some embodiments, the attenuation of light is caused by the scattering of light, such scattering originating in processes such as refraction and/or reflection of light. Further in these and other preferred embodiments attenuation is caused by the absorption of light, such absorption being caused by chemical constituents of the spots under assessment and/or from other agents which are intentionally added to the sample. Accordingly, absorption may be caused by a reagent added to the sample.

In other embodiments, the signal from individual spots or substantially liquid compartments with reaction product is enhanced, e.g. observed increase in light intensity signal relative to light intensity from the background. This can be caused by processes such as scatter, interference, reflection and refraction, typically in combination with focusing or other alteration of the light signal, the enhancement being the result of spatial re-distribution of light. In these embodiments the light relating to a spot or a substantially liquid compartment with reaction product is higher in intensity than the signal from the background and on the bases of this it is possible to process the image in a manner where the signal from individual spots or substantially liquid compartments with reaction product and signal from the background are distinct from each other, preferably where the signal from the spot or substantially liquid compartment with reaction product is substantially higher than the signal from the background, preferable signal from the background in close spatial proximity to the particle.

In some embodiments of the present invention, the recorded image of light intensity from spots or substantially liquid compartments with reaction product in the hydrogel comprises signals relating to spots or substantially liquid compartments with reaction product in the hydrogel, such images comprise change in light intensity information which is a combination of attenuation and enhancement of light intensity relative to the light intensity from the background.

In some embodiments, light from a light source passed through the wall part of the sample compartment has general orientation that is substantially perpendicular to the surface of the wall part. In another preferred embodiment of the present invention, the light from the light source is substantially parallel as it passes the sample. In yet another preferred embodiment of the present invention, the collimated light having passed the sample is substantially focused to a plane located between focusing means and array of detection elements where modulation means are placed substantially in the focus plane. As light from a light source is preferably guided by the use of one or more optical component(s) such as a lens(es) and/or mirror(s), this orientation can be regarded as the optical axis of the light source and in that context the optical axis of the light source is substantially perpendicular to the surface of wall part. Further in several embodiments it is often preferred that the wall parts of the sample compartment are substantially perpendicular to the direction of view of the array of active detection elements. Similarly, as light is generally focused onto the array of active detection elements through the use of optical components such as lens(es) and/or mirror(s), this corresponds to that the optical axis of the array of detection elements is perpendicular to the wall parts. Thus several preferred embodiments of the present invention comprise a light source, sample compartment and array of active detection elements that are all substantially located on a single axis, such that the optical axis of a light source and the array of detection elements are substantially on a single axis, this axis passing through the sample compartment such that its wall parts are substantially perpendicular to this common axis.

In some embodiments, it is included two or more light sources for the illumination of the sample. The two or more light sources typically differ in properties, such as arrangement of optical axis or wavelength. One preferred embodiment of the invention including two or more light sources is where a light from a second light source is passed through wall part of the sample compartment, where the second light source is arranged in a manner such that the main direction of light enters through wall part defining an exposing area of the sample compartment at an angle, preferably at an angle of between 10 and 80 degrees relative to perpendicular. Such arrangement offers typically advantage when two or more light sources are mounted at a substantially permanent location, in particular when more than two light sources are to be mounted. Further it has been found that such angular arrangement can reduce intensity of background light signal. Further, several preferred embodiments of the present invention include three or more, such as four light sources illuminating and/or passing light through the wall parts of the sample compartment. Other equally preferred embodiments include more than four light sources, such as five light sources. As the increasing number of light sources offer the possibility of performing an assessment of spots or substantially liquid compartments with reaction product, where the processing is based on plurality of light intensity information, embodiments including six, seven, eight or even more than eight such as ten individual light sources are preferred.

Here, and elsewhere in the present discussion, the term “main direction of light” can be interchanged with the term “optical axis”, which typically is formed by arranging a light source, or another active component such as an array of active detection elements, in combination with optical means such as one or more and/or mirror(s), (es), irrespective of the function of the lens(es) and/or mirror(s) whether it be for collimation, focusing or dispersion. The optical axis of such a system generally has an axis of symmetry.

In some embodiments, two or more individual light sources emit substantially identical light, for instance with respect to wavelength, preferably in situation such as where such two or more light sources are operated in substantial synchronization and thus increasing the total light energy emitted, and/or extending the illuminated area of the hydrogel and/or sample compartment, and/or assuring more homogeneous light intensity illumination of at least a part of the hydrogel and/or sample compartment. In these but also in other equally preferred embodiments of the present invention two or more individual light sources emit substantially different light, for instance with respect to wavelength, preferably in situation such as where such two or more light sources are operated independently, such as only one such light source being turned on at a time, or such that only the light of one such light source is illuminating the hydrogel and/or sample compartment at a time. One preferred property of such two or more light sources emitting different light is that at least one of such light sources give rise to fluorescence, preferably where such fluorescence intensity information is used in combination with attenuated light intensity information for the purpose of assessing at least one quantity parameter and/or at least one quality parameter of the sample.

In some embodiments, all the light sources are located at the same side of the sample compartment. In such embodiments it is often preferred that such light sources are located at the opposite side of the sample compartment from the array of active detection elements.

Often it is preferred that two or more light sources are arranged in such a manner that light sources giving rise to fluorescence are placed at an angle to the wall parts of the sample compartment, such that the optical axis of the light source are substantially not perpendicular to the wall parts of the sample compartment. One preferred advantage of such an arrangement is the increase in the ratio of the intensity of fluorescence light emitted from biological particles to the intensity of light from the background, often termed Signal to Background (S/B). Light from the background, where background typically is/are region(s) in the image of light intensity information outside any spot or substantially liquid compartment with reaction product of interest, can originate from of a number of sources and/or phenomena some of which are directly related to the properties of the light source, such as the orientation of the light source relative to the optical axis of the array of detection elements. Another equally preferred advantage of arranging a light source at an angle to the wall parts of the sample compartment is that it is possible to locate a number of light sources at a fixed position relative to the sample compartment, thus allowing illumination of the sample compartment with light from two or more light sources without use of mechanical means. Preferred advantage of such properties is/are more simple construction of a system and/or the ability to operate the system faster, when the task is to illuminate the spots or substantially liquid compartments with reaction product in the hydrogel in the sample compartment with two or more different wavelengths in sequence. Further it has been found that such arrangement of excitation light source reduces internal reflection from the light emitting onto a plan surface of the light source, which otherwise can be reflected back onto the array of active detection elements.

One embodiment of the present invention is the use of a light source for the recording of attenuation of light, for instance through refraction and/or reflection. Often it is preferred that the light from such a light source is transmitted through the hydrogel and/or sample compartment in a substantially collimated manner, such that a substantial portion of the light is parallel or substantially parallel. It has been found that such substantially parallel light can enhance the contrast of the attenuation, which is the ratio of attenuation of light by the spot or substantially liquid compartment with reaction product to the intensity of transmitted light in a region of the background. In these and other preferred embodiments of the present invention a portion of the light transmitted through the hydrogel is not exactly parallel, but preferably at an angle less than 45° relative to parallel, such as less than 30°, such as 15°. Even less divergence, such as 10° or less, is often preferred, such as divergence of light of no more than 5° relative to the optical axis.

In preferred implementation of the present invention the optical magnification of light exposed onto an array of active detection element is less than 20:1, defined as the ratio of size of projection of any dimension of an object in the sample compartment to the size of the object. Other equally preferred embodiments have optical magnification of between 1:1 and 20:1, preferably between 1:1 and 1:10.

While some embodiments of the present invention include means with fixed optical magnification, several other embodiments include means which allow recording of images at two or more optical magnifications, which for instance can be used to facilitate detection of cells at lower magnification and subsequent detail analysis of cells at higher magnification. Some preferred embodiments include means for variable optical magnification, e.g. zoom.

The recorded images may be any type of images. In some embodiments, the images are 3D images.

In some embodiments, the recorded image is processed. The recorded images may be processed using various image processing means. In some embodiments, the image is processed using data processing means and/or artificial intelligence. Processing of the image may improve method specificity and facilitate detection and/or analysis of the reaction products of the mixture/and or hydrogel. Thus, in some embodiments, the data processing improves the detection and/or the specificity of the method.

In some embodiments, the data processing means determining or estimating the number of biological particles located in an individual substantially liquid compartment. In some embodiments, the determination or estimation of the number of biological particles located in an individual compartment is used to determine the number of targets and/or biological particles in the sample.

The images recorded by one or more detection elements may be corrected for systematic or varying bias by the use of a calculating means, the bias correction being accomplished by the use of one or more pre-defined value(s).

The bias correction may be performed by subtracting the results obtained in one or several of other images from a later or prior image, such as an image taken previously of the same hydrogel and/or mixture. Also the image from one or more detection elements may be corrected for intensity by the use of a calculating means, said correction being accomplished by the use of one or more pre-defined value(s), preferably where each pre-defined value is determined on the bases of one or more of any previous measurements. Thus, in some embodiments, the data processing means distinguish partially overlapping areas with reaction product.

For the analysis of any measured signal, it is often necessary to digitalize the signal, in such a way that a given intensity of any signal is transformed into a digital representation. This can be done by having a series of channels, were the information about which of these channels has signal which differs from the other channels determines the intensity, or even by having more than one of these channels forming a combination, preferably in a way similar to binary representation.

In one embodiment the target is a nucleic acid molecule which is bound directly or indirectly to a bead. For example the nucleic acid molecule may be linked to a binding moiety such as biotin which can bind to an avidin/streptavidin containing bead. Likewise the nucleic acid molecule may contain a sequence which hybridizes to a corresponding sequence bound to a bead. A bead to which a target is bound is regarded as a biological particle.

In some embodiments, the biological particle is a bead, such as a magnetic bead, optionally wherein the target is linked to the bead via affinity binding such as biotin-streptavidin/avidin or nucleic acid hybridisation,

The use of beads has advantages in purification of nucleic acids, in particular magnetic beads allow the easy purification. The magnetism can also be used for making sure that all beads are situated at the bottom of the solution before gelling. In that way it is ensured that all particles are at the same focus depth. When the solution is gelled as described herein, the beads end up immobilized in the hydrogel which permits the amplification of a fluorescent product in the vicinity of the bead. The hydrogen limits diffusion of the fluorescent product. Beads and fluorescent beads are easily detected in bright field and fluorescent illumination respectively.

The embodiments involving the use of beads can be used as an alternative to qPCR or rtPCR in much the same way as digital PCR. Instead of measuring a quantitative fluorescent signal, the analysis involves counting the number of beads which result in generation of fluorescent reaction produce and dividing this with the total number of beads. The total number of beads can be counted through image analysis in bright field, dark field, or phase contrast illumination.

Instead of performing one reaction per well as in normal PCR, the use of beads involves partitioning the PCR solution into potentially tens of thousands of beads, where a separate PCR reaction takes place on each bead immobilized in the hydrogel. Ideally, the amplification is a loop mediated isothermal amplification (LAMP) amplification. After amplification, the samples are checked for fluorescence with a binary readout of “0” or “1”. The fraction of fluorescing beads is recorded. The partitioning of the sample allows one to estimate the number of different molecules by assuming that the molecule population follows the Poisson distribution, thus accounting for the possibility of multiple target molecules inhabiting a single droplet. Using Poisson's law of small numbers, the distribution of target molecule within the sample can be accurately approximated allowing for a quantification of the target strand in the amplification product. This model simply predicts that as the number of samples containing at least one target molecule increases, the probability of the samples containing more than one target molecule increases. In conventional PCR, the number of PCR amplification cycles is proportional to the starting copy number.

The benefits of using bead partitioning of nucleic acids include increased precision through massive sample partitioning, which ensures reliable measurements in the desired DNA sequence due to reproducibility.

Kit

Provided herein is a kit for detecting a biological particle in a sample, said kit comprising:
a. polymer hydrogel reagents; and
b. nucleic acid amplification, such as isothermal amplification reagents, the reagents being capable of generating a reaction product in the vicinity of particles having a specific target.

Also provided herein is a kit for detecting a biological sample, said kit comprising:
a. polymer hydrogel reagents; and
b. additional reagents, the reagents being capable of generating a reaction product in the vicinity of particles having a specific target.

In some embodiments, the isothermal amplification reagents are as defined in the section “Target in biological particle” and/or anywhere else herein, and the hydrogel reagents are as defined in the section “Hydrogel” and/or anywhere else herein.

In some embodiments, the additional reagents are labelling reagents as defined herein in the sections “Patterned gel” and “Target in biological particle”. Thus, in some embodiments, the additional reagents comprise reagents as defined in the section “Patterned gel”, “Target in biological particle” and/or anywhere else herein, and the hydrogel reagents are as defined in the section “Hydrogel” and/or anywhere else herein.

In some embodiments, the kit further comprises a cellular stain such as a cellular stain for labelling whole cell or parts of cells, and/or a lysing agent such as a lysing agent for lysing cells.

The kit may also comprise instructions for use of the kit. The instructions may be included in a leaflet. The instructions may comprise a description of the order of adding the components to the sample as well as a description of the amount of the components to be used when performing the method. The instructions may also comprise a description of how to assess the results obtained.

The kit may also comprise a cassette or a sample compartment for performing the reactions between the sample and the reagents and/or for analyzing the reacted sample. The cassette or sample compartment may be as described elsewhere herein.

System

In some embodiments, the present invention comprises a system for detecting a biological particle in a sample, said system comprising:
a. a holder for a cassette with a sample compartment,
b. image forming means capable of forming an image of at least part of the sample in the sample compartment on image acquisition means,
c. image processing means,
d. at least one illumination source capable of illuminating the sample in the sample compartment, optionally with a light pattern to induce gelling in illuminated parts thereby creating a hydrogel with a number of substantially liquid compartments separated by substantially gelled parts; and
e. thermostatically controlled heating means capable of heating and optionally cooling the sample in the sample compartment.

The imaging means of the system may be any imaging and/or image acquisition means as described herein in the section “Detection and analysis”.

The thermostatically controlled heating means of the system may be any type of heating and optionally cooling means, such as for example a thermostatically controlled heating and optionally device.

The at least one illumination source of the system may be capable of for example inducing curing of the hydrogel and/or of illuminating the mixture and/or hydrogel to enable recording of one or more images. Thus, in some embodiments, the at least one illumination source is capable of providing specific energy of 0.001 J/cm²-500 J/cm² such as 0.01 to 400 J/cm², for example 0.1 to 300 J/cm², such as 1 to 100 J/cm², for example 10-50 J/cm². In other embodiments, said illumination source is capable of providing specific energy of at least 0.001 J/cm², such as at least 0.01 J/cm², such as at least 0.1 J/cm², such as at least 0.2 J/cm², such as at least 0.25 J/cm², such as at least 0.3 J/cm², such as at least 0.5 J/cm², such as at least 0.75 J/cm², such as at least 0.1 J/cm², such as at least 0.5 J/cm², such as at least 1 J/cm², such as at least 5 J/cm², such as at least 10 J/cm², such as at least 15 J/cm², such as at least 20 J/cm², such as at least 25 J/cm², such as at least 30 J/cm², such as at least 50 J/cm², such as at least 75 J/cm² such as at least 100 J/cm², such as at least 150 J/cm², such as at least 200 J/cm², such as at least 250 J/cm², such as at least 300 J/cm², such as at least 350 J/cm², such as at least 400 J/cm², such as at least 450 J/cm². In some embodiments, the illumination source is capable of providing electromagnetic radiation with a wavelength of at least 100 nm, such as at least 150 nm, such as at least 200 nm, such as at least 250 nm, such as at least 270 nm, such as at least 300 nm, such as at least 350 nm, such as at least 365 nm, such as at least 375 nm, such as at least 380 nm, such as at least 385 nm, such as at least 390 nm, such as at least 395 nm, such as at least 400 nm, such as at least 444 nm, such as at least 450 nm, such as at least 500 nm, such as at least 524 nm, such as at least 550 nm, such as at least 600 nm, such as at least 650 nm, such as at least 700 nm, such as at least 750 nm, such as at least 800 nm, such as at least 850 nm, such as at least 900 nm, such as at least 950 nm. In some embodiments, one illumination source is capable of inducing hydrogel formation, and at least one other illumination source is capable of illuminating the sample to generate an image of at least part of the sample on the image acquisition means.

In some embodiments, the system comprises at least two illumination sources, such as at least three, four, five, six, seven, or eight illumination sources, such as excitation light sources, bright field light sources, such as for example an ultraviolet (UV) light source.

In some embodiments, the image forming means is capable of forming a bright field image, a dark field image, and/or a phase contrast image.

In some embodiments, the heating means such as the thermostatically controlled heating means comprise a heating device configured for heating the sample to a temperature of 40-70° C.

In some embodiments, the illumination source is capable of illuminating the sample with a light pattern to induce gelling in illuminated parts thereby creating a hydrogel with a number of substantially liquid compartments separated by substantially gelled parts.

The system may be used to identify any type of biological particle. In some embodiments, the biological particle is a cell, and the system further comprises means for identifying and isolating cells comprising the target from the substantially liquid compartments. In some embodiments, said cells are selected from the group consisting of bacteria, yeast, protozoa, fungus, plant cells, mammalian cells and insect cells, and the target is selected from the group consisting of a nucleic acid, an exosome, a plasmid, an RNA molecule, an extrachromosomal DNA (ecDNA), an extrachromosomal circular DNA (eccDNA), an aptamer, a nucleic acid tag linked to an aptamer, a molecular beacon, a nucleic acid tag linked to an antibody or a receptor ligand, an antibody, a protein and an enzyme.

In some embodiments, the system comprises a robotic platform for isolating cells from the substantially liquid compartments.

### EXAMPLES

### Example 1—Enumeration of DNA Molecules by gLAMP in UV-Cured Hydrogel

Material and Methods

25 μl gLAMP reaction mix was prepared; LAMP master mix (E1700S, New England Biolabs), LAMP primers see table 1; 0.2 μM (SEQ ID NO:1), 0.2 μM B3 (SEQ ID NO:2), 1.6 μM FIB-FAM (SEQ ID NO:5), 1.6 μM BIP (SEQ ID NO:6), 0.4 μM LF (SEQ ID NO:3), 0.4 μm LB (SEQ ID NO:4), 3.2 μM qFIP-3′-BHQ1 (SEQ ID NO:7), 0.05% Lithium phenyl-2,4,6-trimethylbenzoylphosphinate (LAP, Sigma 900889), 3% 3.4K PEGDA (46497, Alfa Aesar), 7% 20K 4-ARM PEGDA (JKA7034, Sigma) and expression vector EEV604A-2 (0.5 mg/ml, System Biosciences, LLC). The gLAMP reaction mix was loaded onto a Xcyto 6-Chamber slide (Chemometec) and the gel was cured under 395 nm light (70 W electrical power) for 15 seconds prior incubation at 65° C. for 1 hour. Images were acquired with the Xcyto 10 image cytometer (Chemometec) at 4× magnification using the UV bright field channel and a fluorescent channel (excitation: 488 nm, emission: 534 nm). XcytoView (Chemometec) was used for image analysis, scaling and pseudo-coloring.

Results

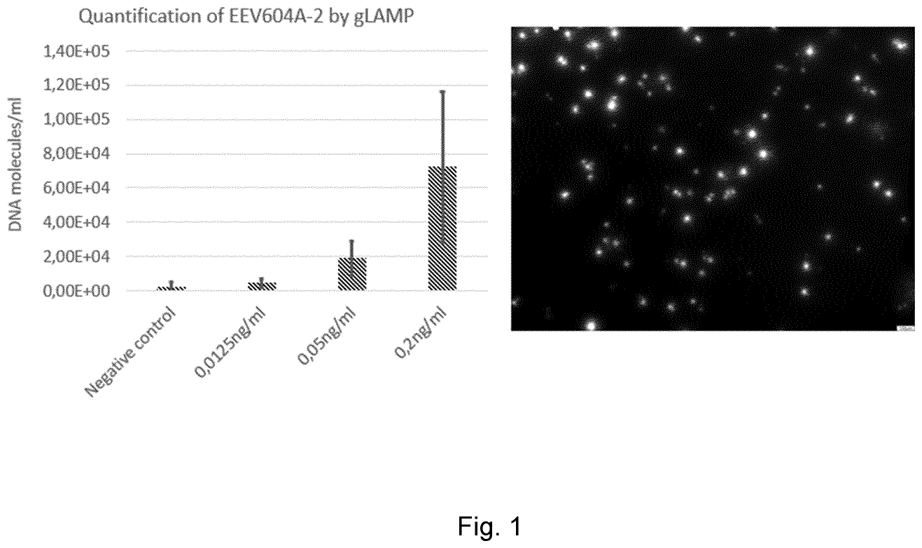

A UV-cured PEGDA gel facilitate a quantitative measurement of the DNA vector EEV604A-2, see FIG. 1. Hence, gLAMP enables detection of DNA in a UV cured hydrogel and allows direct enumeration of DNA molecules in a defined volume.

TABLE 1
LAMP primers used for detection of EEV604.

| LAMP | | |
|---|---|---|
| primers | 5′→3′ sequence | SEQ ID NO. |
| F3 | GTG GTG GAC AGC CAC ATG | SEQ ID NO: 1 |
| B3 | AGA TCC GGT GGA GCC G | SEQ ID NO: 2 |
| LF | GGC GGA AGG CGA ACA TG | SEQ ID NO: 3 |
| LB | CCC ATT GCC TTC GCC AGA | SEQ ID NO: 4 |
| FIB-FAM | Fam-TGT GCA GCT CCT CCA CGC TTT TGC CAT | SEQ ID NO: 5 |
| | CCA CCC CAG CAT | |
| BIP | CTG GGC ATC GTG GAG TAC CAG TTT TGG CAG | SEQ ID NO: 6 |
| | AAT TGG ACG ACT GAG | |
| qFIP-3′- | GAG GAG CTG CAC A-BHQ-1 | SEQ ID NO: 7 |
| BHQ1 | | |

### Example 2—Enumeration of RNA Molecules by gLAMP in UV-Cured Gel

Material and Methods

Escherichia coli bacteriophage MS2 (ATCC® 15597-B1™) was maintained according to the protocol provided from ATCC. MS2 concentration was determined by counting Plaque Forming Units (PFU). In brief, the host strain Escherichia coli C3000 (ATCC® 15597™) was grown over night and spread onto agar plates recommended for cultivating E. coli C3000 by ATCC. The plates were left to dry for 30 minutes, and serial dilutions of the MS2 phage were seeded onto the plates containing the E. coli C3000. The plates were incubated over night at 37 C and the next day PFU were counted. 25 μl gLAMP reaction mix was prepared; LAMP master mix (E1700S, New England Biolabs), LAMP primers (Huang et al., 2018); 0.2 μM F3, 0.2 μM B3, 1.6 μM FIB-FAM, 1.6 μM BIP, 0.4 μM LF, 0.4 μm LB, 3.2 μM qFIP-3′-BHQ1, 0.05% Lithium phenyl-2,4,6-trimethylbenzoylphosphinate (LAP, Sigma 900889), 3% 3.4K PEGDA (46497, Alfa Aesar), 7% 20K 4-ARM PEGDA (JKA7034, Sigma) and MS2 sample. The gLAMP reaction mix was loaded onto a Xcyto 6-Chamber slide (Chemometec) and the gel was cured under 395 nm light (70 W electrical power) for 15 seconds prior incubation at 65 C for 1 hour. Images were acquired with the Xcyto 10 image cytometer (Chemometec) at 4× magnification using the UV bright field channel and a fluorescent channel (excitation: 488 nm, emission: 534 nm). XcytoView (Chemometec) was used for image analysis, scaling and pseudo-coloring.

Results

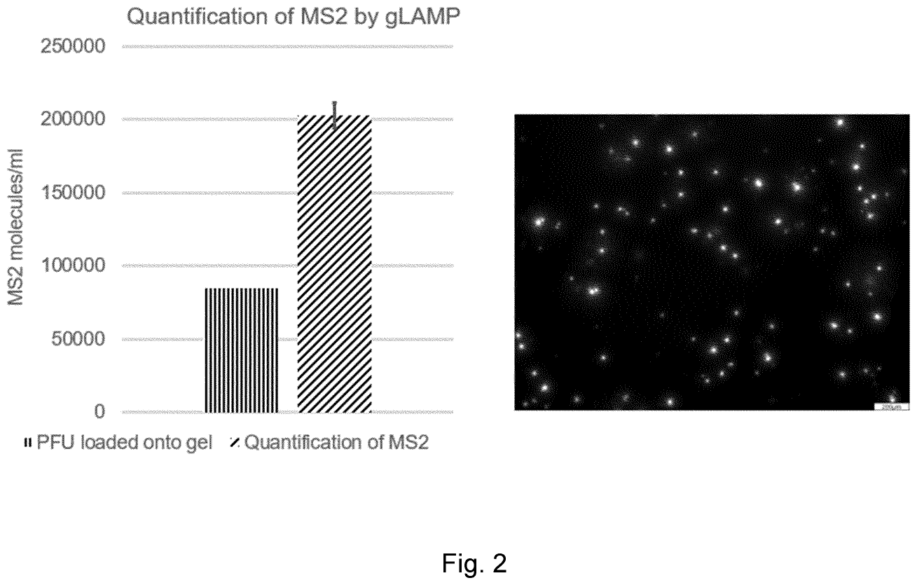

A UV-cured PEGDA hydrogel facilitates a quantitative measurement of the RNA virus MS2. The PEGDA hydrogel-based LAMP method provides a 2-fold higher count than the PFU count, see FIG. 2. This discrepancy can be explained by the fact that gLAMP detects all viruses containing RNA genome, while the PFU method only detects plaque forming virus particles. Hence, non-viable virus particles are excluded in the PFU count. In conclusion, gLAMP enables detection of RNA in a UV cured hydrogel and allows direct enumeration of RNA molecules and RNA based viruses in a defined volume.

### Example 3—Time-Course Imaging Increases Dynamic Range and Eliminates False Positives

Material and Methods

25 μl gLAMP reaction mix was prepared; LAMP master mix (E1700S, New England Biolabs), LAMP primers see table 2; 0.2 μM F3(SEQ ID NO:8), 0.2 μM B3 (SEQ ID NO:9), 1.6 μM FIB-FAM (SEQ ID NO:12), 1.6 μM BIP (SEQ ID NO:13), 0.4 μM LF (SEQ ID NO:10), 0.4 μm LB (SEQ ID NO:11), 3.2 μM qFIP-3′-BHQ1 (SEQ ID NO:14), 0.05% Lithium phenyl-2,4,6-trimethylbenzoylphosphinate (LAP, Sigma 900889), 3% 6K PEGDA (701963, Sigma), 7% 10K 4-ARM PEGDA (abx085084, Abbexa) and expression vector EEV604A-2 (0.5 mg/ml, System Biosciences, LLC). The gLAMP reaction mix was loaded onto a Xcyto 6-Chamber slide (Chemometec) and the gel was cured under 395 nm light (70 W electrical power) for 15 seconds prior incubation at 65° C. for 0, 30 and 60 minutes. Images were acquired at the different time points with Xcyto 10 image cytometer (Chemometec) at 4× magnification using the UV bright field channel and a fluorescent channel (excitation: 488 nm, emission: 534 nm). XcytoView (Chemometec) was used for image analysis, scaling and pseudo-coloring.

Results

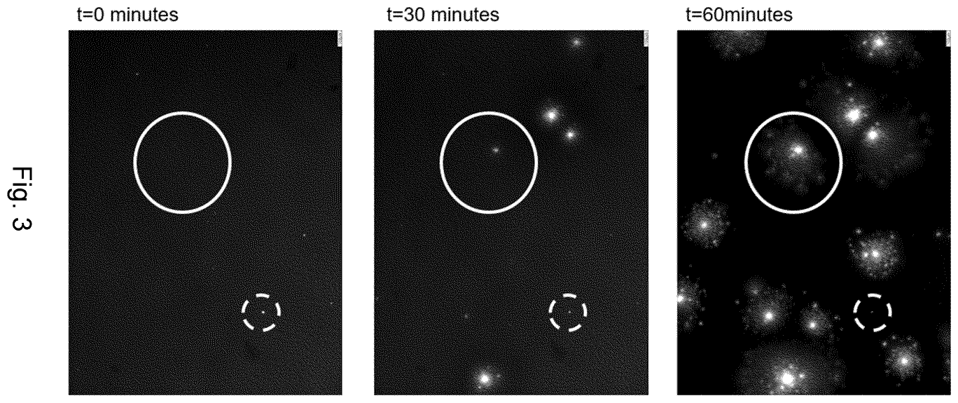

To ensure that all molecules of target nucleic acids in the sample are identified, prolonged incubation time may be necessary, see FIG. 3. However, during long incubation times satellite “colonies” around early emerging amplicons can appear due to diffusion of LAMP products, see full circle in FIG. 3. This phenomenon is not confined to PEGDA gels and has also been described for PEG and polyacrylamide gels (Huang et al., 2018). To avoid counting satellite colonies, we propose to collect images continuously, and combine the information from several images to increase precision and robustness of the quantification. In FIG. 3, all the amplicons within the full circle should only be counted as one. Furthermore, time-course imaging allows identification and exclusion of false positive events, an example of false positive is marked with a dashed circle in FIG. 3. Finally, time-course imaging increases the dynamic range of the assay, since it allows robust identification of early as well as late emerging amplicons. In conclusion, time-course imaging increases the precision and accuracy of gLAMP by eliminating false positive, avoiding counting satellite amplicons and increasing the dynamic range of the assay.

TABLE 2
LAMP primers used for detection of EEV604, adapted from Gadkar et al, 2018.

| LAMP | | |
|---|---|---|
| primers | 5′→3′ sequence | SEQ ID NO. |
| F3 | GCG GCC AAC TTA CTT CTG AC | SEQ ID NO: 8 |
| B3 | CAA CGT TGT TGC CAT TGC TA | SEQ ID NO: 9 |
| LF | TGA AGC CAT ACC AAA CGA CG | SEQ ID NO: 10 |
| LB | TGT TGT GCA AAA AAG CGG TTA G | SEQ ID NO: 11 |
| FIB-FAM | FAM-AGG CGA GTT ACA TGA TCC CCC AGA TCG | SEQ ID NO: 12 |
| | GAG GAC CGA AGG AG | |
| BIP | TGA TCG TTG GGA ACC GGA GCC AGG CAT CGT | SEQ ID NO: 13 |
| | GGT GTC AC | |
| qFIP-3′- | ATG TAA CTC GCC T-BHQ-1 | SEQ ID NO: 14 |
| BHQ1 | | |

### Example 4—Workflow for gLAMP Instrument

Results

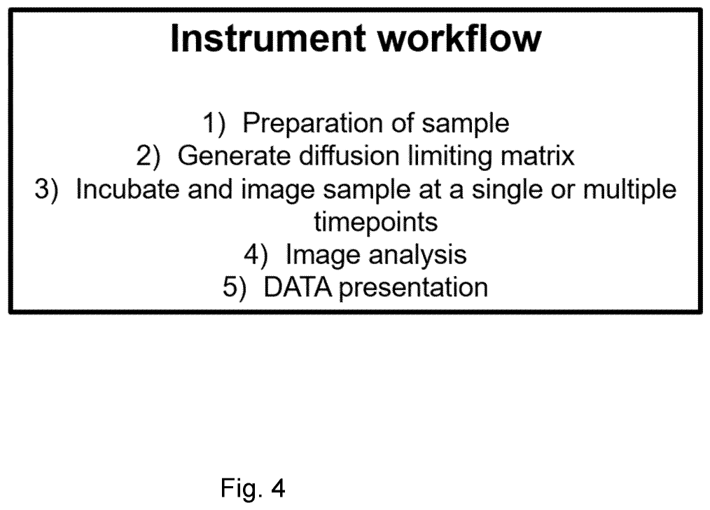

Automatization of sample handling, imaging and analysis minimizes human errors and, thus, deliver more, objective, unbiased, precise, and reproducible results. Furthermore, automation leads to substantial less hands-on time, thereby improving the user experience. We propose an instrument for gLAMP where several steps are automated. The instrument should perform two or more of the steps listed in FIG. 4. A nucleic acid sample mixed with reaction reagents could be loaded directly into the instrument, or via a sample media that are inserted into the instrument. Alternatively, the sample could be loaded directly into the instrument and automatically mixed with the reaction reagents inside the instrument. Generation of a diffusion limiting matrix can be induced by exposure to specific light wavelengths or temperatures inside the instrument. Alternatively, a reaction mix comprising reagents that stochastically will form a matrix could be used. During incubation, imaging of the sample should be possible at a single or at multiple timepoints. Finally, the automated image analysis and data presentation will ensure unbiased interpretation of the fluorescent events, as well as precise and reproducible results with reduced hands-on time.

### Example 5—Detection of Specific DNA Sequence in U2OS Cells

Material and Methods

U2OS cells were maintained at 37° C. in a humidified atmosphere with 5% C02 in RPMI (61870, Gibco) supplemented with 8% heat inactivated FBS (10500, Gibco). U20S cells were transfected with EEV604A-2 using lipofectamine 3000 according to manufacturer's protocol (L3000001, ThermoFisher). Next day transfected and untransfected U20S cells were seeded onto 12-well slides (81201, Ibidi) and incubated overnight. The following day the cells were either fixed in 100% Methanol for 10 min at −20° C., fixed in neutral buffered formalin (NBF) for 15 minutes at room temperature and permeabilized with 0.5% Triton X-100 in PBS for 10 minutes at room temperature, or washed in PBS for live cell analysis. 25 μl gLAMP reaction mix was prepared; LAMP master mix (E1700S, New England Biolabs), LAMP primers (see table 1); 0.2 μM F3 (SEQ ID NO:1), 0.2 μM B3 (SEQ ID NO:2), 1.6 μM FIB-FAM (SEQ ID NO:5), 1.6 μM BIP (SEQ ID NO:6), 0.4 μM LF (SEQ ID NO:3), 0.4 μm LB (SEQ ID NO:4), 3.2 μM qFIP-3′-BHQ1 (SEQ ID NO:7), 0.05% Lithium phenyl-2,4,6-trimethylbenzoylphosphinate (LAP, Sigma 900889), 3% 3.4K PEGDA (46497, Alfa Aesar), 7% 20K 4-ARM PEGDA (JKA7034, Sigma). The gLAMP reaction mix was poured over slides with fixed or live cells and immediately thereafter, the gel was cured under 395 nm light (70 W electrical power) for 15 seconds. The slides were incubated at 65° C. for 1 hours. Images were acquired with the Xcyto 10 image cytometer (Chemometec) at 4× magnification using the UV bright field channel and a fluorescent channel (excitation: 488 nm, emission: 534 nm). XcytoView (Chemometec) was used for image analysis, scaling and pseudo-coloring.

Results

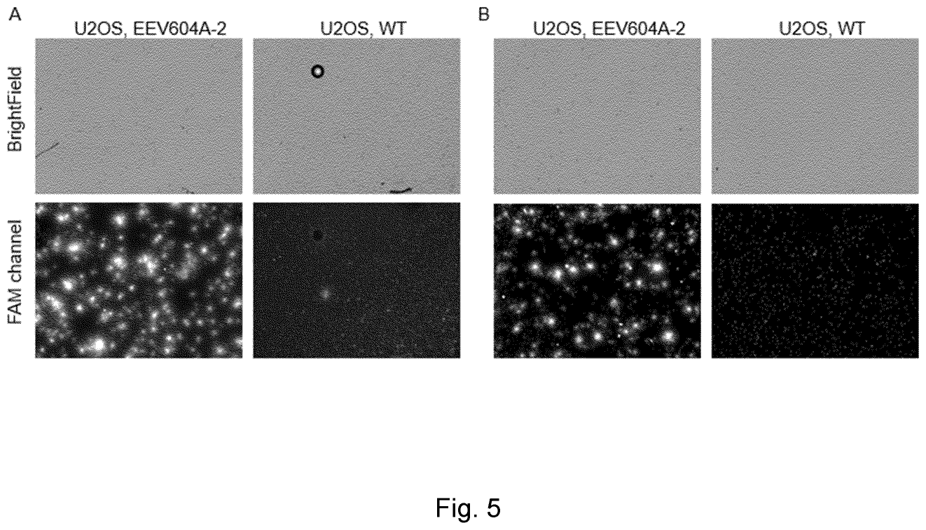

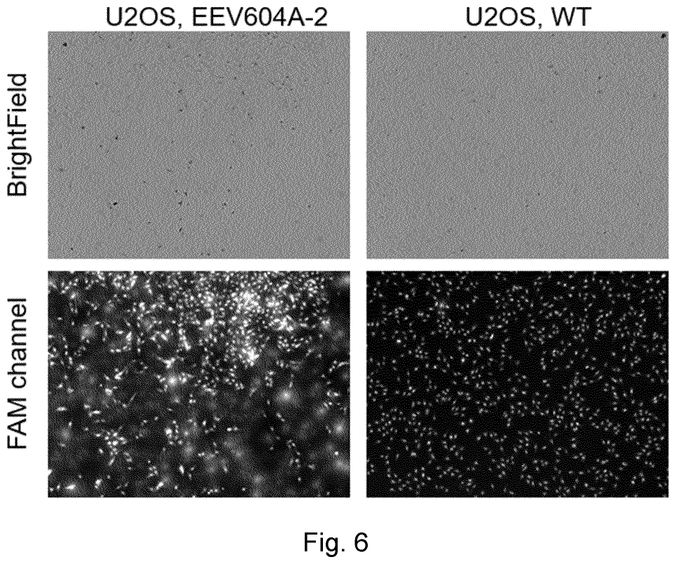

A UV-cured PEGDA hydrogel facilitates detection of the DNA vector EEV604A-2 in both live and fixed U20S cells, see FIGS. 5 and 6. Detection of a specific DNA sequence via gLAMP is a fast and sensitive alternative to conventional methods such as Fluorescence In Situ Hybridization (FISH).

### Example 6—Incorporation of Positively Charged Monomers in Hydrogel for gLAMP

Materials and Methods

25 μl gLAMP reaction mix was prepared; LAMP master mix (E1700S, New England Biolabs), LAMP primers see table 2; 0.2 μM F3 (SEQ ID NO:8), 0.2 μM B3 (SEQ ID NO:9), 1.6 μM FIB-FAM (SEQ ID NO:12), 1.6 μM BIP (SEQ ID NO:13), 0.4 μM LF (SEQ ID NO:10), 0.4 μm LB (SEQ ID NO:11), 3.2 μM qFIP-3′-BHQ1 (SEQ ID NO:14), 0.05% Lithium phenyl-2,4,6-trimethylbenzoylphosphinate (LAP, Sigma 900889), 3% 3.4K PEGDA (46497, Alfa Aesar), 7% 20K 4-ARM PEGDA (JKA7034, Sigma), expression vector EEV604A-2 (0.5 mg/ml, System Biosciences, LLC) with or without 2 mM 2-(methacryloyloxy)ethyl-trimethylammonium chloride (MAETAC, 408107, Sigma). The gLAMP reaction mix was loaded onto a Xcyto 6-Chamber slide (Chemometec) and the gel was cured under 395 nm light (70 W electrical power) for 15 seconds prior incubation at 65° C. for 1 hour. Images were acquired with the Xcyto 10 image cytometer (Chemometec) at 4× magnification using the UV bright field channel and a fluorescent channel (excitation: 488 nm, emission: 534 nm). XcytoView (Chemometec) was used for image analysis, scaling and pseudo-coloring.

Results

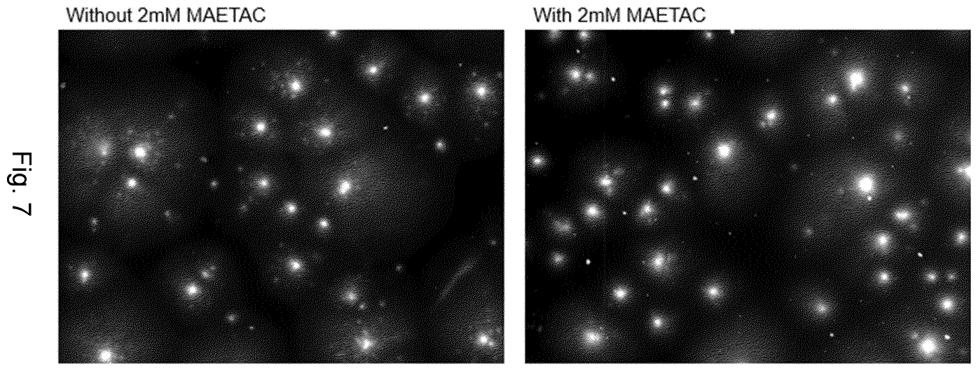

Introducing a positive charge to PEGDA hydrogel could potentially retard the diffusion of negatively charged DNA products in the hydrogel and thereby reduce satellite amplicons. A positive charged hydrogel could as well lead to more distinct amplicons thereby simplifying the image analysis as well as increasing the dynamic range. FIG. 7 demonstrates that incorporation of the positively charged monomer MAETAC into the hydrogel reduces the formation of satellite amplicons in gLAMP.

### Example 7—UV Photolithography Induced μm-Sized Reaction Wells for Digital gLAMP

Materials and Methods

50 μl gLAMP reaction mix was prepared; LAMP master mix (E1700S, New England Biolabs), LAMP primers see table 2; 0.2 μM F3 (SEQ ID NO:8), 0.2 μM B3 (SEQ ID NO:9), 1.6 μM FIB-FAM (SEQ ID NO:12), 1.6 μM BIP (SEQ ID NO:13), 0.4 μM LF (SEQ ID NO:10), 0.4 μm LB (SEQ ID NO:11), 3.2 μM qFIP-3′-BHQ1 (SEQ ID NO:14), 0.05% Lithium phenyl-2,4,6-trimethylbenzoylphosphinate (LAP, Sigma 900889), 5% 2K 4-ARM PEGDA (abx085084, Abbexa), expression vector EEV604A-2 (0.2 ng/ml, System Biosciences, LLC). The gLAMP reaction mix was loaded onto a Xcyto 2-Chamber slide (Chemometec). Reaction wells with a diameter of 100 μm was formed by UV photolithography using a collimated 385 nm light scource (SOLIS-385C, ThorLabs) illuminating through a photolithographic mask for 60 seconds. The slide was incubated at 65° C. for 1 hour. Images were acquired with the Xcyto 10 image cytometer (Chemometec) at 4× magnification using the UV bright field channel and a fluorescent channel (excitation: 488 nm, emission: 534 nm). XcytoView (Chemometec) was used for image analysis, scaling and pseudo-coloring.

Results

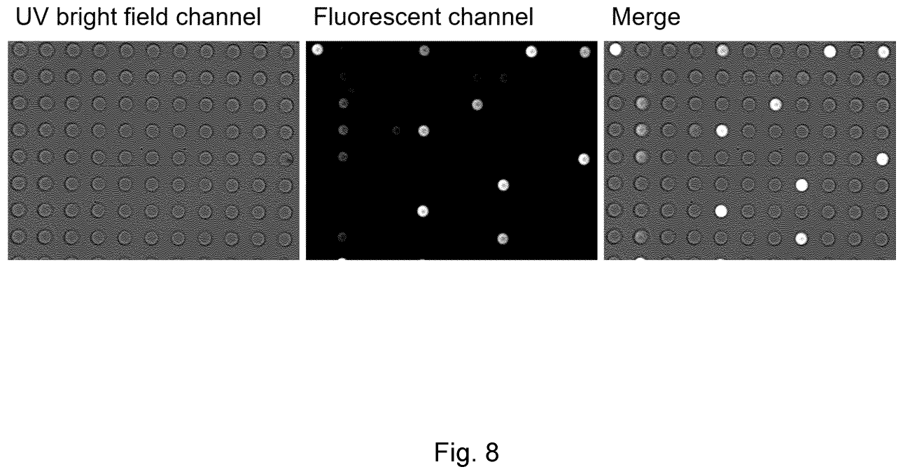

Digital bioassays have a great potential; however, they often require advanced and expensive equipment. We have developed a method for digital gel bioassays, where we use light to create thousands of reaction wells by spatial inducing curing of hydrogel components. A digital gel bioassay can be an easy and robust alternative to the established digital bioassay platforms. To illustrate the applicability of digital gel bioassay, we here demonstrate that a hydrogel with all reaction components can be divided into thousands of reaction wells by photolithography (see FIG. 8, left image). After incubation, a fluorescent signal was generated in the reaction wells where the DNA vector EEV604A-2 was present (see FIG. 8, middle and right images). The digital gLAMP assay generates a digital output that can be used to calculate most probable number of DNA vector EEV604A-2 molecules.

### Example 8—Digital Detection of Immobilized Nucleic Acids by gLAMP

Materials and Methods

gLAMP: 20 μl gLAMP reaction mix was prepared; LAMP master mix (E1700S, New England Biolabs), LAMP primers see table 2; 0.2 μM F3 (SEQ ID NO:8), 0.2 μM B3 (SEQ ID NO:9), 1.6 μM FIP-FAM (SEQ ID NO:12), 1.6 μM BIP (SEQ ID NO:13), 0.4 μM LF (SEQ ID NO:10), 0.4 μm LB (SEQ ID NO:11), 3.2 μM qFIP-3′-BHQ1 (SEQ ID NO:14), 0.05% Lithium phenyl-2,4,6-trimethylbenzoylphosphinate (LAP, Sigma 900889), 7.5% 20K 4-ARM PEGDA (JKA7034, Sigma), Dynabeads™ M-280 Streptavidin (ThermoFisher) loaded with target DNA as described below (final concentration of ˜280 beads/μl). The gLAMP reaction mix was loaded onto a Xcyto 6-Chamber slide (Chemometec) and beads were focused on the bottom of the slide by placing the slide on a DynaMag™-2 Magnet (ThermoFisher) for 1 min. Afterwards the gel was cured under 395 nm light (70 W electrical power) for 15 seconds and the slide was incubated at 65° C. for 1 hour. Images were acquired with the Xcyto 10 image cytometer (Chemometec) at 4× magnification using the UV bright field channel and a fluorescent channel (excitation: 488 nm, emission: 534 nm). XcytoView (Chemometec) was used for image analysis, scaling, and pseudo-coloring.

Immobilization of target DNA: Biotinylated DNA was synthesized by PCR using SsoAdvanced Universal Probes Supermix (Bio-Rad) according to manufacturer protocol. Forward primer (5′biotin-ACGAGAACCCCTTCCTGCACGC, SEQ ID NO:23) and reverse primer (B3, SEQ ID NO:9, see table 2) were commercially obtained (tag Copenhagen) and EEV604A-2 was used as template. The PCR product was purified by agarose gel electrophoresis and subsequent gel extraction (GeneJET Gel Extraction Kit, ThermoFisher). 10 μl Dynabeads™ M-280 Streptavidin (ThermoFisher) were washed once with 100 μl 1× wash buffer (5 mM Tris-HCl (pH 7.4), 0.5 mM EDTA, 1M NaCl) according to manufacturer instructions and incubated with biotinylated DNA (135 pg in 40 μl 1× wash buffer) for 15 min at room temperature. Dynabeads™ were washed twice with 1× wash buffer (100 μl), twice with TE buffer (10 mM Tris-HCl (pH 7.4), 1 mM EDTA 100 μl) and twice with water (100 μl) before being resuspended in TE buffer (40 μl).

Results

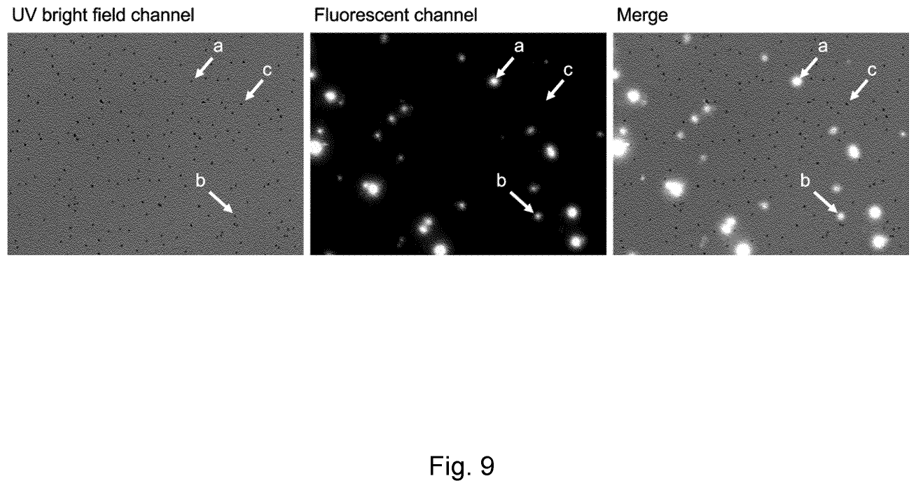

Immobilization is often used in the purification of biological molecules or other analytes. Directly performing a biological assay on the solid support can ease the workflow. Here we demonstrate that gLAMP can be directly performed on magnetic beads serving as a solid support. The magnetic beads can also be used to obtain a digital readout. Magnetic beads with and without immobilized target DNA are cast directly into the PEGDA hydrogel. The beads can be easily optically detected, e.g., in bright field channel (see FIG. 9 left and right image). After incubation, a fluorescent LAMP product is generated in the proximity of beads carrying the target DNA, which is diffusion limited by the hydrogel (see FIG. 9 middle and right image). By counting the fraction of positive beads, the assay generates a digital output that can be used to calculate the most probable number of target DNA immobilized on the beads.

### Example 9—gLAMP Detection of Nucleic Acids with Single Nucleotide Resolution

Materials and Methods

Template DNA: Template DNA containing a fragment of the original tGFP sequence (tGFP wt) was obtained by performing a PCR on EEV604A-2 using PCR Primer 2+3 ((SEQ ID NO:19)+(SEQ ID NO:20), see table 3) and Phusion polymerase (Thermo Fisher) according to manufacturer protocol. Template DNA with a single point mutation at position 155 (tGFP mut) was obtained by performing a PCR on EEV604A-2 using PCR Primer 1+3 (SEQ ID NO:18)+(SEQ ID NO:20) (see table 3), followed by a consecutive PCR using Primer 2+3 (SEQ ID NO:19)+(SEQ ID NO:20). The PCR products were purified by agarose gel electrophoresis and subsequent gel extraction (GeneJET Gel Extraction Kit, ThermoFisher) and were diluted 1:10,000 before being used as template in gLAMP.

gLAMP: 14 μl gLAMP reaction mix was prepared from LAMP master mix (E1700S, New England Biolabs), LAMP primers see table 3; 00.2 μM F3 (SEQ ID NO:1), 0.2 μM B3 (SEQ ID NO:2), 1.6 μM FIP (SEQ ID NO:15), 1.6 μM BIP (SEQ ID NO:6), 0.6 μM LEC-Probe 1 (SEQ ID NO:16), 0.6 μM LEC-Probe 2 (SEQ ID NO:17), 2 μM Syto41, 2U Endonuclease IV (M0304S, New England Biolabs) and 1 μl template DNA. Last, 6 μl of a 3:1 mixture of PEG acrylate (40 kDa 4arms) and thiol PEG (3.4 kDa 2arms) was added to the reaction mix. The gLAMP reaction mix was loaded onto a Xcyto 6-Chamber slide (Chemometec), cured by incubation at room temperature for 5 min, followed by an incubation at 65° C. for 60 min. Images were acquired with the Xcyto 10 image cytometer (Chemometec) at 4× magnification using the UV bright field channel and three fluorescent channels, namely FAM (excitation: 488 nm, emission: 534 nm), Cy5 (excitation: 635 nm, emission: 685 nm) and Syto41 (excitation: 405 nm, emission: 453 nm). XcytoView (Chemometec) was used for image analysis, scaling, and pseudo-coloring.

Results

There are several strategies to achieve single nucleotide discrimination using LAMP (Varona et al., 2021). However only few of them allow precise quantification of two DNA strands with small differences in sequence. LEC-LAMP uses probes that are incorporated in the LAMP amplicon and are cleaved by Endonuclease IV if they perfectly match the template, resulting in an on-switch in fluorescence (Higgins et al., 2020). Importantly the generated signal stays associated to the LAMP amplicon it originates from. While LEO-LAMP in solution has potential for multiplexing and allows for single nucleotide discrimination, it is not suited to precisely quantify two similar DNA species, especially, if one of them is present in strong excess. We demonstrate here that combining the use of two LEO-probes with the gLAMP strategy creates two distinct populations of amplicons with differential cleaving of the probes, thereby enabling single molecule enumeration combined with single nucleotide resolution. The combination of those features has a high potential in diagnostic applications, such as liquid biopsies or strain specific pathogen detection.

TABLE 3
LAMP primers used for LEC-LAMP, PCR Primers for generating templates and
full sequence of template PCR Products with the mutation site highlighted

| LAMP primers | 5′→3′ sequence | SEQ ID NO. |
|---|---|---|
| F3 | GTGGTGGACAGCCACATG | SEQ ID NO: 1 |
| B3 | AGATCCGGTGGAGCCG | SEQ ID NO: 2 |
| FIP | TGTGCAGCTCCTCCACGCTTTTGCCATCCAC | SEQ ID NO: 15 |
| | CCCAGCAT | |
| BIP | CTGGGCATCGTGGAGTACCAGTTTTGGCAG | SEQ ID NO: 6 |
| | AATTGGACGACTGAG | |
| LEC-probe 1 | BHQ1-AGAC(dSpacer)C(C-FAM) | SEQ ID NO: 16 |
| | CATTGCCTTCGCCAGA | |
| LEC-probe 2 | BHQ2-AGAT(dSpacer)C(C-Cy5) | SEQ ID NO: 17 |
| | CATTGCCTTCGCCAGA | |
| PCR Primer 1 | CACGGCAGAATTGGACGACTGAGCGCGGG | SEQ ID NO: 18 |
| | ATCTGGCGAAGGCAATG | |
| | GGGATCTTGAAGGC | |
| PCR Primer 2 | ACAGGTACCGTGGTGGACAGCCACATGCAC | SEQ ID NO: 19 |
| | TTCAAGAGCGCCATCC ACCCCAGCAT | |
| PCR Primer 3 | AGAATTCAGATCCGGTGGAGCCGGGTCCGG | SEQ ID NO: 20 |
| | CGGTGCCGTCCACGGC AGAATTGGACGAC | |
| tGFP wt | ACAGGTACCGTGGTGGACAGCCACATGCAC | SEQ ID NO: 21 |
| PCR product | TTCAAGAGCGCCATCCACCCCAGCATCCTG | |
| | CAGAACGGGGGCCCCATGTTCGCCTTCCGC | |
| | CGCGTGGAGGAGCTGCACAGCAACACCGA | |
| | GCTGGGCATCGTGGAGTACCAGCACGCCTT | |
| | CAAGACCCCCATTGCCTTCGCCAGATCCCG | |
| | CGCTCAGTCGTCCAATTCTGCCGTGGACGG | |
| | CACCGCCGGACCCGGCTCCA | |
| | CCGGATCTGAATTC | |
| tGFP mut | ACAGGTACCGTGGTGGACAGCCACATGCAC | SEQ ID NO: 22 |
| PCR product | TTCAAGAGCGCCATCCACCCCAGCATCCTG | |
| | CAGAACGGGGGCCCCATGTTCGCCTTCCGC | |
| | CGCGTGGAGGAGCTGCACAGCAACACCGA | |
| | GCTGGGCATCGTGGAGTACCAGCACGCCTT | |
| | CAAGATCCCCATTGCCTTCGCCAGATCCCG | |
| | CGCTCAGTCGTCCAATTCTGCCGTGGACGG | |
| | CACCGCCGGACCCGGCTCCA | |
| | CCGGATCTGAATTC | |
| Forward primer- | ACGAGAACCCCTTCCTGCACGC | SEQ ID NO: 23 |
| example 8 | | |

### Example 10—Enumeration of DNA Molecules by gMDA (Multiple Displacement Amplification) in UV-Cured Hydrogel

Material and Methods

50 μl gMDA reaction mix was prepared; 25U Phi29 (M0269SVIAL, New England Biolabs), 1× Phi29 Buffer (B0269SVIAL, New England Biolabs), 1 mM dNTPs (NU-1006S, Jena Bioscience) 5 μM SYBR14 (L-7011, Molecular Probes) 0.05% Lithium phenyl-2,4,6-trimethylbenzoylphosphinate (LAP, Sigma 900889), 5% 10K 4-ARM PEGDA (Abx085084, Abbexa). Expression vector EEV604A-2 (2 ng/ml, System Biosciences, LLC) was denatured by 20 mM NaOH for 3 minutes in the presence of 20 μM Exo-Resistant Random Primer (SO181, Thermo Fischer) and reannealed by 20 mM HCl. The Expression vector EEV604A-2 with primers was mixed with the gMDA reaction and loaded onto a Xcyto 2-Chamber slide (Chemometec). The hydrogel was cured under a 385 nm light source (SOLIS-385C, ThorLabs) for 60 seconds prior incubation at 30° C. for 16 hours. Images were acquired with the Xcyto 10 image cytometer (Chemometec) at 4× magnification using the UV bright field channel and a fluorescent channel (excitation: 488 nm, emission: 534 nm).

Results

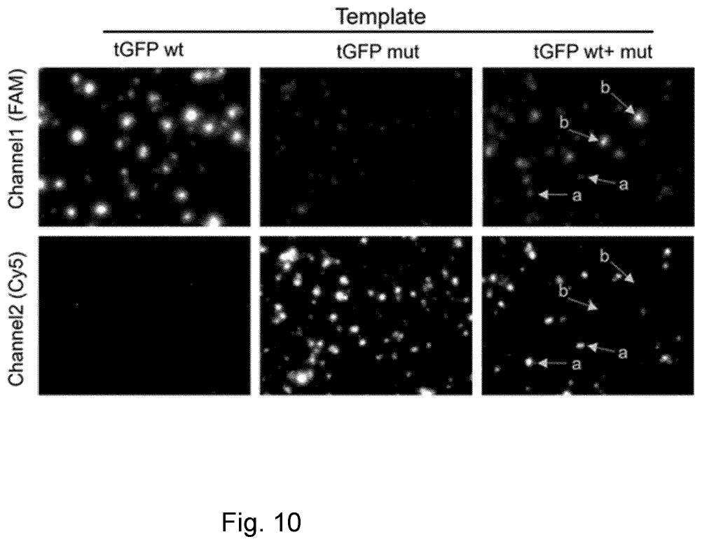

A UV-cured PEGDA hydrogel facilitate detection of the DNA vector EEV64A-2 by MDA (gMDA), see FIG. 10. Hence, gMDA enables detection of DNA in a UV cured hydrogel and allows direct enumeration of DNA molecules in a defined volume.

Sequences

| SEQ ID NO | Sequence |
|---|---|
| SEQ ID NO: 1 | GTG GTG GAC AGC CAC ATG |
| SEQ ID NO: 2 | AGA TCC GGT GGA GCC G |
| SEQ ID NO: 3 | GGC GGA AGG CGA ACA TG |
| SEQ ID NO: 4 | CCC ATT GCC TTC GCC AGA |
| SEQ ID NO: 5 | Fam-TGT GCA GCT CCT CCA CGC TTT TGC CAT CCA CCC CAG |
| | CAT |
| SEQ ID NO: 6 | CTG GGC ATC GTG GAG TAC CAG TTT TGG CAG AAT TGG ACG |
| | ACT GAG |
| SEQ ID NO: 7 | GAG GAG CTG CAC A-BHQ-1 |
| SEQ ID NO: 8 | GCG GCC AAC TTA CTT CTG AC |
| SEQ ID NO: 9 | CAA CGT TGT TGC CAT TGC TA |
| SEQ ID NO: | TGA AGC CAT ACC AAA CGA CG |
| 10 | |
| SEQ ID NO: | TGT TGT GCA AAA AAG CGG TTA G |
| 11 | |
| SEQ ID NO: | FAM-AGG CGA GTT ACA TGA TCC CCC AGA TCG GAG GAC CGA |
| 12 | AGG AG |
| SEQ ID NO: | TGA TCG TTG GGA ACC GGA GCC AGG CAT CGT GGT GTC AC |
| 13 | |
| SEQ ID NO: | ATG TAA CTC GCC T-BHQ-1 |
| 14 | |
| SEQ ID NO: | TGTGCAGCTCCTCCACGCTTTTGCCATCCACCCCAGCAT |
| 15 | |
| SEQ ID NO: | BHQ1-AGAC(dSpacer)C(C-FAM)CATTGCCTTCGCCAGA |
| 16 | |
| SEQ ID NO: | BHQ2-AGAT(dSpacer)C(C-Cy5)CATTGCCTTCGCCAGA |
| 17 | |
| SEQ ID NO: | CACGGCAGAATTGGACGACTGAGCGCGGGATCTGGCGAAGGC |
| 18 | AATG GGGATCTTGAAGGC |
| SEQ ID NO: | ACAGGTACCGTGGTGGACAGCCACATGCACTTCAAGAGCGCCA |
| 19 | TCC ACCCCAGCAT |
| SEQ ID NO: | AGAATTCAGATCCGGTGGAGCCGGGTCCGGCGGTGCCGTCCA |
| 20 | CGGC AGAATTGGACGAC |
| SEQ ID NO: | ACAGGTACCGTGGTGGACAGCCACATGCACTTCAAGAGCGCCA |
| 21 | TCCACCCCAGCATCCTGCAGA |
| | ACGGGGGCCCCATGTTCGCCTTCCGCCGCGTGGAGGAGCTGC |
| | ACAGCAACACCGAGCTGGGCATCGTGGAGTACCAGCACGCCTT |
| | CAAGACCCCCATTGCCTTCGCCAGATCCCGCGCTCAGTCGTCC |
| | AATTCTGCCGTGGACGGCACCGCCGGACCCGGCTCCA |
| | CCGGATCTGAATTC |
| SEQ ID NO: | ACAGGTACCGTGGTGGACAGCCACATGCACTTCAAGAGCGCCA |
| 22 | TCCACCCCAGCATCCTGCAGAACGGGGGCCCCATGTTCGCCTT |
| | CCGCCGCGTGGAGGAGCTGCACAGCAACACCGAGCTGGGCAT |
| | CGTGGAGTACCAGCACGCCTTCAAGATCCCCATTGCCTTCGCC |
| | AGATCCCGCGCTCAGTCGTCCAATTCTGCCGTGGACGGCACCG |
| | CCGGACCCGGCTCCA CCGGATCTGAATTC |

### REFERENCES

Ahmed. Hydrogel: Preparation, characterization, and applications: A review. J Adv Res. 6(2), p 105-121 (2015).
Gadkar et al. Real-time Detection and Monitoring of Loop Mediated Amplification (LAMP) Reaction Using Self-quenching and De-quenching Fluorogenic Probes. Sci Rep 8, 5548 (2018). doi.org/10.1038/s41598-018-23930-1
Higgins, O., & Smith, T. J. (2020). Loop-primer endonuclease cleavage-loop-mediated isothermal amplification technology for multiplex pathogen detection and single-nucleotide polymorphism identification. The Journal of Molecular Diagnostics, 22(5), 640-651. https://doi.org/10.1016/j.jmoldx.2020.02.002
Huang et al. Smartphone-Based in-Gel Loop-Mediated Isothermal Amplification (gLAMP) System Enables Rapid Coliphage MS2 Quantification in Environmental Waters. Environ Sci Tech. 52(11), p. 6399-6407 (2018).
Varona, M., & Anderson, J. L. (2021). Advances in Mutation Detection Using Loop-Mediated Isothermal Amplification. ACS omega, 6(5), 3463-3469. https://doi.org/10.1021/acsomega.0c06093
Verhaak et al. Extrachromosomal oncogene amplification in tumour pathogenesis and evolution. Nat Rev Cancer. 19(5), p. 283-288 (2019).
Zhang et al. A semi-synthetic organism that stores and retrieves increased genetic information. Nature. 551(7682), p. 644-647 (2017).
Zhu et al. A hydrogel beads based platform for single-cell phenotypic analysis and digital molecular detection. bioRxiv 848168 (2019). https://doi.org/10.1101/848168

Items 1

1. A method of detecting or analyzing a biological particle in a sample, said method comprising the steps of:
a. mixing the sample with polymer hydrogel reagents and isothermal amplification reagents, the isothermal amplification reagents being capable of generating a reaction product in the vicinity of particles having a specific target;
b. inducing curing of the mixture to a hydrogel;
c. incubating the mixture or hydrogel, whereby one or more reaction products are produced as a result of the isothermal amplification reaction;
d. recording one or more images of the hydrogel.

2. The method according to item 1, wherein steps b-d can be conducted in any order, preferably wherein step b is conducted before step c.

3. The method according to any one of the preceding items, wherein the reagents comprise a primer set and optionally an aptamer, a molecular beacon aptamer, an antibody, a probe, or a receptor ligand with a nucleotide tag.

4. A method of detecting or analyzing a biological particle in a sample, said method comprising the steps of:
a. mixing the sample with curable polymer hydrogel reagents, the curable polymer hydrogel reagents comprising a photoinitiator;
b. illuminating the sample with a light pattern to induce gelling in illuminated parts thereby creating a number of substantially liquid compartments separated by substantially gelled parts;
c. detecting or analyzing particles in the substantially liquid compartments.

5. A method of creating compartments in a sample with biological particles, said method comprising the steps of:
a. mixing the sample with curable polymer hydrogel reagents, the curable polymer hydrogel reagents comprising a photoinitiator;
b. illuminating the sample with a light pattern to induce gelling in illuminated parts thereby creating a number of substantially liquid compartments separated by substantially gelled parts.

6. The method of items 4-5, wherein the pattern is created by inserting a mask in the light path between the light source and the sample, or wherein the pattern is created by illuminating the sample with a laser.

7. A method for detecting or analyzing extrachromosomal DNA (ecDNA) and/or extrachromosomal circular DNA (eccDNA), the method comprising the steps of:
a. mixing isothermal amplification reagents, curable polymer hydrogel reagents, and sample comprising ecDNA and/or eccDNA, the isothermal amplification reagents being capable of generating a reaction product in the vicinity of the ecDNA and/or eccDNA;
b. curing the mixture to a hydrogel;
c. incubating the mixture or hydrogel, whereby one or more amplicons are produced as a result of the isothermal amplification reaction amplifying whole or part of the ecDNA and/or eccDNA, if said ecDNA and/or eccDNA is present in the sample;
d. recording one or more images of the hydrogel.

8. The method according to item 7, wherein steps b-d can be conducted in any order, preferably wherein step b is conducted before step c.

9. The method according to any one of items 7-8, wherein the extrachromosomal DNA is or contains a marker of cancer, such as an oncogene or a marker for a genetic disease or an autoimmune disease.

10. The method according to any one of items 7-9, wherein the extrachromosomal DNA is a plasmid.

11. A method for detecting or analyzing viral particles in a sample, the method comprising the steps of:
a. mixing isothermal amplification reagents, curable polymer hydrogel reagents, and sample comprising viral particles, the isothermal amplification reagents being capable of generating a reaction product in the vicinity of the viral particles;
b. curing the mixture to a hydrogel;
c. incubating the mixture or hydrogel, whereby one or more amplicons are produced as a result of the isothermal amplification reaction amplifying whole or part of the nucleic acids present in the viral particle, if said viral particle is present in the sample;
d. recording one or more images of the hydrogel.

12. The method according to item 11, wherein steps b-d can be conducted in any order, preferably wherein step b is conducted before step c.

13. The method according to any one of the preceding items, wherein a cellular stain is mixed with the reagents prior to curing the hydrogel.

14. The method according to any one of the preceding items, wherein curing is induced by a change in temperature, application of a current, by radiation, or by pressure.

15. The method according to any one of the preceding items, wherein curing is done in more than one step, such as in two steps.

16. The method according to any one of the preceding items, wherein the duration of the step of curing the mixture to a hydrogel is less than 30 minutes, preferably less than 10 minutes, preferably less than 5 minutes, preferably less than 1 min, preferably less than 30 seconds.

17. The method according to any one of the preceding items, wherein the polymer hydrogel is prepared from chemicals selected from the group consisting of acrylics, methacrylics, acrylamides, and styrenics.

18. The method according to any one of the preceding items, wherein the polymer hydrogel reagents comprise a polymer/monomer selected from the group consisting of polyacrylamide (PA), polyethylene glycol (PEG), polyethylene glycol diacrylate (PEGDA), dimethacrylated PEG (PEGDM), Poly(ethylene glycol)-block-polylactide methyl ether (PEG-b-PLA), Poly-D,L-lactic acid/polyethylene glycol/poly-D,L-lactic acid (PDLLA-PEG), Poly(vinyl alcohol) (PVA), Chondroitin sulfate, Chitosan, Heparin, Dextran, Agarose, Alginate, Starch, Pectin, Polyvinylamine, Polyphosphazene, Poly(L-glutamic-acid), poly(N,N-dimethylacrylamide-co-furfuryl methacrylate), carboxymethylcellulose, poly(N-isopropylacrylamide), poly(aspartic acid), gellan gum, copoly(acrylamide), hydroxypropylmethylcellulose, collagen-inspired undecapeptide, collagen, fibrin, gelatin, silk fibroin, silk-MA, hyaluronic acid, methacrylated hyaluronic acid, methacrylated gelatin, methacrylated alginate, methacrylated collagen, and DNA.

19. The method according to any one of the preceding items, wherein the polymer hydrogel reagents comprise polyethylene glycol diacrylate (PEGDA) with a molecular weight between 0.5-100K, such as 3.4 K, such as 5 K, such as 10 K, such as 15 K, such as 20 K, such as 25 K.

20. The method according to any one of the preceding items, wherein the pore size of the hydrogel is between 0.1 nm-1000 nm, such as 0.5 nm, such as 1 nm, such as 5 nm, such as 10 nm, such as 15 nm, such as 16 nm, such as 17 nm, such as 18 nm, such as 19 nm, such as 20 nm, such as 21 nm, such as 22 nm, such as 23 nm, such as 24 nm, such as 25 nm, such as 30 nm, such as 40 nm, such as 50 nm, such as 100 nm, such as 200 nm, such as 300 nm, such as 400 nm, such as 500 nm, such as 700 nm, such as 900 nm.

21. The method according to any one of the preceding items, wherein the pore size of the hydrogel is less than 0.1 nm.

22. The method according to any one of the preceding items, wherein the concentration of monomer/polymer in the mixture is between 0.1%-40% (w/v), such as 0.5% (w/v), such as 0.75% (w/v), such as 1% (w/v), such as 2% (w/v), such as 3% (w/v), such as 4% (w/v), such as 5% (w/v), such as 6% (w/v), such as 7% (w/v), such as 8% (w/v), such as 9% (w/v), such as 10% (w/v), such as 11% (w/v), such as 13% (w/v), such as 15% (w/v), such as 17% (w/v), such as 19% (w/v) such as 20% (w/v), such as 25% (w/v), such as 30% (w/v), such as 35% (w/v).

23. The method according to any one of the preceding items, wherein the hydrogel is heterogeneous.

24. The method according to any one of the preceding items, wherein the hydrogel contains two or more areas with different properties, such as areas with different pore size, density, and/or different degree of solidity.

25. The method according to any one of the preceding items, wherein the hydrogel contains two or more areas with different properties, such as areas of high degree of solidity and areas of substantially no solidity.

26. The method according to any one of the preceding items, wherein a pattern is made in the hydrogel, such as a pattern which generates multiple compartments.

27. The method according to item 26, wherein the compartments are μlaced in rows in the hydrogel.

28. The method according to any one of items 26-27, wherein the compartments are between 50-5000μ², such as 100μ², such as 200μ², such as 300μ², such as 400μ², such as 500μ², such as 600μ², such as 700μ², such as 800μ², such as 900μ², such as 1000μ², such as 2000μ², such as 3000μ², such as 4000μ².

29. The method according to any one of the preceding items, wherein an agent is added to the mixture in order to make the hydrogel positively charged.

30. The method according to item 29, wherein the agent is a positively charged polymer, such as poly-lysine or polyethylenimine.

31. The method according to item 29, wherein the agent is a positively charged monomer, such as 2-(methacryloyloxy)ethyl-trimethylammonium chloride (MAETAC), butyl methacrylate or [2-(acryloyloxy)ethyl]trimethylammonium chloride (AETAC).

32. The method according to any one of items 1-25, wherein an agent is added to the mixture in order to make the hydrogel negatively charged.

33. The method according to item 32, wherein the agent is a negatively charged polymer, such as DNA, polystyrene sulfonate and polyacrylic acid.

34. The method according to item 32, wherein the agent is a negatively charged monomer, such as sodium 2-sulfoethyl methacrylate (SEMA).

35. The method according to any one of the preceding items, wherein the hydrogel is incubated at a temperature between 20° C. to 80° C., such as 25° C., such as 30° C., such as 35° C., such as 37° C., such as 40° C., such as 45° C., such as 50° C., such as 55° C., such as 60° C., such as 65° C., such as 70° C., such as 75° C.

36. The method according to any one of the preceding items, wherein the polymer hydrogel reagents comprise a photoinitiator.

37. The method according to item 36, wherein the photoinitiator is selected from the group consisting of lithium phenyl-2,4,6-trimethylbenzoylphosphinate (LAP), Irgacure 651, Irgacure 184, Irgacure 907, Irgacure 2959 (12959), Diphenyl(2,4,6-trimethylbenzoyl)phosphine oxide (TPO), phenylbis (2,4,6-trimethylbenzoyl) phosphine oxide (BAPO), BAPO-OLi, BAPO-ONa, VA-086, Eosin Y, Riboflavin, Camphorquinone, 1,4-bis(4-(N,N-bis(6-(N,N,N-trimethylammonium)hexyl)amino)-styryl)-2,5-dimethoxybenzene tetraiodide (WSPI), 2,5-bis-[4-(diethylamino)-benzylidene]-cyclopentanone (BDEA), 3,3′-((((1E,1′E)-(2-oxocyclopentane-1,3-diylidene)bis(methanylylidene))bis(4,1 phenylene))bis(methylazanediyl))dipropanoate (P2CK), and ruthenium.

38. The method according to item 36, wherein the photoinitiator is lithium phenyl-2,4,6-trimethylbenzoylphosphinate (LAP) or Irgacure 2959 (12959).

39. The method according to any one of items 36-38, wherein the concentration of photoinitiator in the mixture is between 0.0001%-5% w/w, such as 0.0005% w/w, such as 0.001% w/w, such as 0.005% w/w, such as 0.01% w/w, such as 0.05% w/w, such as 0.1% w/w, such as 0.2% w/w, such as 0.3% w/w, such as 0.4% w/w, such as 0.5% w/w, such as 0.6% w/w, such as 0.07% w/w, such as 0.08% w/w, such as 0.9% w/w, such as 1% w/w, such as 1.5% w/w, such as 2% w/w, such as 2.5% w/w, such as 3% w/w, such as 3.5% w/w, such as 4% w/w, such as 4.5% w/w.

40. The method according to any one of items 36-39, wherein the mixture is cured to a hydrogel at a wavelength between 100 nm-1200 nm, such as 150 nm, such as 200 nm, such as 250 nm, such as 270 nm, such as 300 nm, such as 350 nm, such as 365 nm, such as 375 nm, such as 380 nm, such as 385 nm, such as 390 nm, such as 395 nm, such as 400 nm, such as 444 nm, such as 450 nm, such as 500 nm, such as 524 nm, such as 550 nm, such as 600 nm, such as 650 nm, such as 700 nm, such as 750 nm, such as 800 nm, such as 850 nm, such as 900 nm, such as 950 nm.

41. The method according to any one of items 36-40, wherein the mixture is cured to a hydrogel at an intensity of 0.001 J/cm²-500 J/cm², such as 0.01 J/cm², such as 0.1 J/cm², such as 0.2 J/cm², such as 0.25 J/cm², such as 0.3 J/cm², such as 0.5 J/cm², such as 0.75 J/cm², such as 0.1 J/cm², such as 0.5 J/cm², such as 1 J/cm², such as 5 J/cm², such as 10 J/cm², such as 15 J/cm², such as 20 J/cm² such as 25 J/cm², such as 30 J/cm², such as 50 J/cm², such as 75 J/cm², such as 100 J/cm², such as 150 J/cm², such as 200 J/cm², such as 250 J/cm², such as 300 J/cm², such as 350 J/cm², such as 400 J/cm², such as 450 J/cm².

42. The method according to any one of the preceding items, wherein a current is used to cure the mixture to a hydrogel.

43. The method according to item 42, wherein the hydrogel is cured with a current after being cured by any of the curing methods according to any one of the preceding items.

44. The method according to item 42, wherein curing the hydrogel with a current decreases the diffusion rates in the hydrogel.

45. The method according to any one of the preceding items, wherein the polymer hydrogel reagents comprise an agent which enables the hydrogel to be cured by a change in temperature.

46. The method according to item 45, wherein the agent is selected from the group consisting of bovine serum albumin (BSA), laminin, type IV collagen, heparan sulfate proteoglycan and/or entactin.

47. The method according to any one of items 45-46, wherein the hydrogel is cured by increasing the temperature between 20° C.-50° C., such as 25° C., such as 30° C., such as 35° C., such as 40° C., such as 45° C.

48. The method according to any one of the preceding items, wherein the biological particle is a target nucleic acid and the reaction product is a nucleic acid amplified from said target nucleic acid.

49. The method according to any one of the preceding items, wherein the isothermal amplification reagents comprise reagents for rolling circle amplification (RCA), loop mediated isothermal amplification (LAMP), multiple displacement amplification (MDA), nucleic acid sequence-based amplification (NASBA), helicase-dependent amplification (HDA), recombinase polymerase amplification (RPA), whole genome amplification (WGA), self-sustained sequence replication (3SR), transcription mediated amplification (TMA), signal-mediated amplification of RNA technology (SMART), ramification amplification (RMA), proximity ligation assay (PLA) and/or strand displacement amplification (SDA).

50. The method according to any one of the preceding items, wherein the isothermal amplification reagents comprise
a. a DNA polymerase, such as Phi29 or mutant versions thereof such as EquiPhi29 or QualiPhi; BST or mutant versions thereof such as large fragment, BST 2.0, BST 3.0 or IsoPol BST; BSU; BSM; IsoPol SD+;
and/or Klenow; and
b. dNTPs; and optionally,
c. a reverse transcriptase.

51. The method according to any one of the preceding items, wherein the reagents further comprise dimethyl sulfoxide (DMSO), Propyl sulfoxide, Methyl sec-butyl sulfoxide, Tetramethylene Sulfoxide, Methyl sulfone, Sulfolane, single-stranded DNA binding protein (SSB), TaqSSB, T4 gene 32 protein (gp32), PEG4000, PEG400, Non-ionic detergents (such as, Tween-20, Triton X-100, NP-40), Glycerol, Propylene glycol, Ethylene glycol, Betaine, Trehalose, Formamide, Acetamide, 2-pyrrolidone, Tetramethyl ammonium chloride (TMAC), TMA oxalate, TMA hydrogen sulfate, TEA-CI, TPA-CI, TPA-acetate, TBA-CI, 7-deaza-2′-deoxyguanosine, Magnesium, Dithiothreitol (DTT), pyrophosphatase and/or bovine serum albumin (BSA).

52. The method according to any one of the preceding items, wherein the isothermal amplification reagents further comprise magnesium pyrophosphate, calcium pyrophosphate, hydroxynaphthol blue or calcein.

53. The method according to item 52, wherein magnesium or calcium pyrophosphate is deposited in the vicinity of said biological particle as a result of the isothermal amplification reaction.

54. The method according to any one of the preceding items, wherein the isothermal amplification reagents further comprise a dye to label the amplified nucleic acid.

55. The method according to any one of the preceding items, wherein the dye is linked to a primer or to deoxynucleotide triphosphates (dNTPs) which are incorporated during the isothermal amplification.

56. The method according to any one of items 54-55, wherein the dye is an intercalating dye or a fluorescent tagged probe.

57. The method according to item 56, wherein the intercalating dye is SYBR Green, SYBR Gold, LAMP Dye, DAPI, Hoechst, ToPro3, Draq5, Draq7, RedDot1, RedDot2, Propidium iodide, Ethidium bromide and/or Evagreen.

58. The method according to item 56, wherein the fluorescent probe is FITC, FAM, PE, APC, Cy3, Cy5, Cy7, PerCP, AF488, AF647, AF555, CF488 CF555, CF647, any of the fluorescent probes from the Alexa fluorophore family, any of the fluorescent probes from the Biotium fluorophore family, any of the fluorescent probes from the Atto fluorophore family, quantum dots, and/or a polymer dye from the Brilliant violet dye family.

59. The method according to any one of the preceding items, wherein the multiple displacement amplification (MDA) or loop mediated isothermal amplification (LAMP) reagents comprise a primer-dye and a primer-quencher set.

60. The method according to any one of the preceding items, wherein the biological particle is a biological cell, a viral particle, a bacteriophage, a bead, an exosome, a plasmid, an extrachromosomal DNA, or an extrachromosomal circular DNA.

61. The method according to item 60, wherein the biological particle is a bead, such as a magnetic bead, optionally wherein the target is linked to the bead via affinity binding such as biotin-streptavidin/avidin or nucleic acid hybridisation,

62. The method according to any one of the preceding items, wherein the target is a nucleic acid in a virus, a viral particle, a bacteriophage, a bacterium, a protozoan, a yeast, a fungus, a plant cell, an insect cell, a mammalian cell, an exosome, a plasmid, an extrachromosomal DNA (ecDNA), an extrachromosomal circular DNA (eccDNA), an aptamer, a nucleic acid tag linked to an aptamer, a molecular beacon, a nucleic acid tag linked to an antibody or a receptor ligand, or residual nucleic acid in pharmaceutical products.

63. The method according to item 62, wherein the nucleic acid is DNA or RNA, such as viral RNA or mRNA.

64. The method according to item 62, wherein the mammalian cell is a cell from a non-human cell line.

65. The method according to item 62, wherein the mammalian cell is a cell from a human.

66. The method according to any one of the preceding items, wherein the nucleic acid is a medical marker of a disease, such as an oncogene or a marker of a genetic disease or an autoimmune disease.

67. The method according to any one of the preceding items, wherein the target is a protein and the method comprises adding a reagent capable of binding said protein, the reagent comprising a nucleic acid tag, such as a DNA tag.

68. The method according to item 67, wherein the reagent comprises an aptamer, a molecular beacon aptamer, an antibody, or a receptor ligand.

69. The method according to any one of the preceding items, wherein the reaction product is a product that causes reflection, refraction, diffraction, interaction, scattering or absorbance of light emitted from a light source onto the sample.

70. The method according to any one of the preceding items, wherein the reaction product is a fluorescent molecule, or where a fluorescent molecule can bind to the reaction product.

71. The method according to any one of the preceding items, wherein the reaction product is coloured.

72. The method according to any one of the preceding items, wherein the sample comprises viruses, viral particles, bacteriophages, bacteria, protozoa, yeast, fungi, plant cells, insect cells, mammalian cells, exosomes, proteins, enzymes, plasmids, DNA, RNA, mRNA, microRNA, extrachromosomal DNA (ecDNA), extrachromosomal circular DNA (eccDNA), and/or residual nucleic acid in pharmaceutical products.

73. The method according to item 72, wherein the mammalian cells are from a non-human cell line.

74. The method according to item 72, wherein the mammalian cells are human cells.

75. The method according to any one of the preceding items, wherein the sample comprises viruses, bacteria, yeast, mammalian cells, plasmids, exosomes, extrachromosomal DNA (ecDNA), extrachromosomal circular DNA (eccDNA), DNA, RNA, mRNA, microRNA and/or residual DNA in pharmaceutical products.

76. The method according to any one of the preceding items, wherein the sample is a body fluid sample, a tissue sample, a fermentation sample, a liquid cultivation sample, a cell culture sample, a water sample, a beverage sample, a pharmaceutical sample, an environmental sample, a sewage sample, a diagnostic sample or a sample of a pharmaceutical product.

77. The method according to item 76, wherein the body fluid sample is selected from the group consisting of samples from swabs, blood, plasma, serum, urine, bile, saliva, semen, cerebrospinal fluid and mucus.

78. The method according to item 76, wherein the tissue sample is selected from the group consisting of tissue samples from liver, kidney, muscle, brain, lung, skin, thymus, spleen, gastrointestinal tract, pancreas and thyroid gland.

79. The method according to any one of items 77-78, wherein the sample is a sample from a human.

80. The method according to any one of the preceding items, further comprising counting the number of particles and the number of particles with reaction product in at least part of the sample.

81. The method according to any one of the preceding items, further comprising quantifying the amount, such as the concentration, of target, by counting the amount of spots present in the hydrogel.

82. The method according to item 81, wherein the number of spots is counted in an image using image analysis to identify spots.

83. The method according to any one of the preceding items, further comprising recording an image of the hydrogel or mixture before incubation and/or induction of hydrogel formation.

84. The method according to item 83, wherein said image is used to determine the background of the hydrogel or mixture before incubation.

85. The method according to any one of the preceding items, wherein multiple images of the hydrogel are recorded while the hydrogel is being incubated, such as wherein images are compared following image processing to determine the number of spots.

86. The method according to item 85, wherein the multiple images are used to evaluate the formation of reaction product over time.

87. The method according to any one of the preceding items, wherein the recording of images comprises the use of a confocal scanner, a microscope, a fluorescence microscope, an image cytometer, and/or an automated particle counter.

88. The method according to any one of the preceding items, wherein the recording of images comprises the use of one or more optical channels.

89. The method according to any one of the preceding items, wherein the recording of images comprises the use of one or more fluorescent channels.

90. The method according to any one the preceding items, wherein the images are recorded using an array of detection devices.

91. The method according to any one the preceding items, wherein the images are recorded using a two-dimensional array of detection devices.

92. The method according to any one of the preceding items, wherein the images are 3D images.

93. The method according to any one of the preceding items, wherein the images are recorded using a CCD, a CMOS, a video camera, photomultiplier tubes, photodiode, avalanche photodiode (APD) or a photon counting camera.

94. The method according to any one of the preceding items, wherein the recorded image is processed.

95. The method according to item 94, wherein the recorded image is processed using data processing means.

96. The method according to item 95, wherein the image is processed using artificial intelligence.

97. The method according to any one of items 95-96, wherein the data processing means distinguish partially overlapping areas with reaction product.

98. The method according to any one of items 95-97, wherein the data processing improves the detection and/or the specificity of the method.

99. The method according to any one of the preceding items, wherein sample is loaded into a sample compartment, such as wherein the mixture is cured inside said sample compartment.

100. The method according to item 99, wherein the sample compartment forms part of a cassette.

101. The method according to any one of items 99-100, wherein the sample compartment is defined by two parallel sheets of transparent material, preferably plastic material or glass material with a given distance between them.

102. The method according to any one of items 99-101, wherein the cassette comprises channels for loading the sample.

103. The method according to any one of items 99-102, wherein the cassette comprises channels for flowing the sample within the cassette.

104. The method according to any one of items 99-103, wherein the cassette comprises a valve for regulating the flow of sample.

105. The method according to any one of items 99-104, wherein at least one wall of the sample compartment is transparent.

106. The method according to any one of items 99-105, wherein the cassette is removable.

107. The method according to any one of items 99-106, wherein the cassette is single-use and/or disposable.

108. The method according to any one of items 99-107, wherein the photoinitiator is preloaded into the sample compartment or into a channel part of the cassette.

109. The method according to any one of the preceding items, wherein a cellular stain is added to the mixture prior to curing the hydrogel.

110. The method according to item 109, wherein the cellular stain is a stain which stains the whole cell or a part of the cell, such as the cell membrane, the cell wall, the cytoplasm, the nucleus, the mitochondria and/or nucleic acids.

111. The method according to any one of the preceding items, wherein a lysing agent is added to the mixture prior to curing the hydrogel.

112. The method according to any one of the preceding items, wherein the sample is pre-treated using a method comprising one or more of the following steps:
a. incubating the sample at 98° C. for 5 minutes; and/or
b. mixing the sample with 96% ethanol; and/or
c. mixing the sample with 1-1000 mM sodium hydroxide; and/or
d. mixing the sample with detergent, such as NP40, tween20 or triton X100; and/or
e. mixing the sample with saponin; and/or
f. mixing the sample with 96% methanol.

113. A kit for detecting a biological particle in a sample, said kit comprising:
a. polymer hydrogel reagents; and
b. isothermal amplification reagents, the reagents being capable of generating a reaction product in the vicinity of particles having a specific target.

114. The kit according to item 113, wherein the isothermal amplification reagents comprise reagents as defined in any one of items 3, 49-59 and 68, and wherein the hydrogel reagents are as defined in any one of items 17-19, 22, 29-31, 36-39 and 45-46.

115. The kit according to item 113, further comprising a cellular stain.

116. The kit according to item 113, further comprising a lysing agent.

117. A system for detecting a biological particle in a sample, said system comprising:
a. a holder for a cassette with a sample compartment,
b. image forming means capable of forming an image of at least part of the sample in the sample compartment on image acquisition means,
c. image processing means,
d. at least one illumination source capable of illuminating the sample in the sample compartment; and
e. thermostatically controlled heating means capable of heating the sample in the sample compartment.

118. The system according to item 117, wherein at least one illumination source is capable of providing specific energy of at least 0.001 J/cm², such as at least 0.01 J/cm², such as at least 0.1 J/cm², such as at least 0.2 J/cm², such as at least 0.25 J/cm², such as at least 0.3 J/cm², such as at least 0.5 J/cm², such as at least 0.75 J/cm², such as at least 0.1 J/cm², such as at least 0.5 J/cm², such as at least 1 J/cm², such as at least 5 J/cm², such as at least 10 J/cm², such as at least 15 J/cm², such as at least 20 J/cm², such as at least 25 J/cm², such as at least 30 J/cm², such as at least 50 J/cm², such as at least 75 J/cm², such as at least 100 J/cm², such as at least 150 J/cm², such as at least 200 J/cm², such as at least 250 J/cm², such as at least 300 J/cm², such as at least 350 J/cm² such as at least 400 J/cm², such as at least 450 J/cm².

119. The system according to item 117, wherein the illumination source is capable of providing electromagnetic radiation with a wavelength of at least 100 nm, such as at least 150 nm, such as at least 200 nm, such as at least 250 nm, such as at least 270 nm, such as at least 300 nm, such as at least 350 nm, such as at least 365 nm, such as at least 375 nm, such as at least 380 nm, such as at least 385 nm, such as at least 390 nm, such as at least 395 nm, such as at least 400 nm, such as at least 444 nm, such as at least 450 nm, such as at least 500 nm, such as at least 524 nm, such as at least 550 nm, such as at least 600 nm, such as at least 650 nm, such as at least 700 nm, such as at least 750 nm, such as at least 800 nm, such as at least 850 nm, such as at least 900 nm, such as at least 950 nm.

120. The system according to item 117, wherein one illumination source is capable of inducing hydrogel formation, and at least one other illumination source is capable of illuminating the sample to generate an image of at least part of the sample on the image acquisition means.

121. The system according to any one of items 117-119, wherein the system comprises at least two illumination sources, such as at least 3, 4, 5, 6, 7, or 8 illumination sources, such as excitation light sources, bright field light sources, such as for example a UV light source.

122. The system according to any of items 117-121, wherein the image forming means is capable of forming a bright field image, a dark field image, and/or a phase contrast image.

123. The system according to any of items 117-122, wherein the heating means comprise a heating device configured for heating the sample to a temperature of 40-70° C.

Items 2

1. A method of detecting or analyzing a biological particle in a sample, said method comprising the steps of:
a. mixing the sample with curable polymer hydrogel reagents, the curable polymer hydrogel reagents comprising a photoinitiator;
b. illuminating the sample with a light pattern to induce gelling in illuminated parts thereby creating a hydrogel with a number of substantially liquid compartments separated by substantially gelled parts;
c. detecting or analyzing particles in the substantially liquid compartments.

2. The method according to item 1, wherein labelling reagents are provided together with the hydrogel reagents, the labelling reagents being capable of generating a reaction product in the vicinity of particles having a specific target.

3. The method according to any one of the preceding items, wherein the labelling reagents comprise:
a. a primer set and optionally an aptamer, a molecular beacon aptamer, an antibody, a probe, or a receptor ligand with a nucleotide tag;
b. enzyme assay reagents, such as enzyme substrate, enzyme and/or enzyme co-factors; and/or
c. a protein linked to a nucleic acid tag, such as a DNA tag, and/or an antibody.

4. The method according to any one of the preceding items, wherein said method further comprises the step of incubating the hydrogel to allow formation of the reaction products.

5. The method according to any one of the preceding items, wherein each substantially liquid compartment comprise a discrete number of biological particles, such as 0, 1, 2, 3, 4, 5, 6, 7, 8, 9 or 10 particles, preferably 0, 1, 2, or 3 particles.

6. The method according to any one of the preceding items, wherein the pattern is created by inserting a mask in the light path between the light source and the sample, or wherein the pattern is created by illuminating the sample with a light source that forms a light spot, whereby a predetermined pattern is projected across at least part of the sample.

7. The method according to any one of the preceding items, wherein the liquid compartments are placed in rows in the hydrogel.

8. The method according to any one of the preceding items, wherein the pattern is created using UV photolithography, wherein a collimated light source illuminates the sample through a photolithographic mask.

9. The method according to any one of the preceding items, wherein the duration of the step of illuminating the sample is less than 15 minutes, preferably less than 10 min, preferably less than 8 min, preferably less than 6 min, preferably less than 4 min, preferably less than 2 min, preferably less than 1 min, preferably less than 30 seconds.

10. The method according to any one of the preceding items, wherein the mixture is illuminated at a wavelength between 100 nm-1200 nm, such as 150 nm, such as 200 nm, such as 250 nm, such as 270 nm, such as 300 nm, such as 350 nm, such as 365 nm, such as 375 nm, such as 380 nm, such as 385 nm, such as 390 nm, such as 395 nm, such as 400 nm, such as 444 nm, such as 450 nm, such as 500 nm, such as 524 nm, such as 550 nm, such as 600 nm, such as 650 nm, such as 700 nm, such as 750 nm, such as 800 nm, such as 850 nm, such as 900 nm, such as 950 nm.

11. The method according to any one of the preceding items, wherein the mixture is illuminated at an intensity of 0.001 J/cm²-500 J/cm², such as 0.01 J/cm², such as 0.1 J/cm², such as 0.2 J/cm², such as 0.25 J/cm², such as 0.3 J/cm², such as 0.5 J/cm², such as 0.75 J/cm², such as 0.1 J/cm², such as 0.5 J/cm², such as 1 J/cm², such as 5 J/cm², such as 10 J/cm², such as 15 J/cm², such as 20 J/cm² such as 25 J/cm², such as 30 J/cm², such as 50 J/cm², such as 75 J/cm², such as 100 J/cm², such as 150 J/cm², such as 200 J/cm², such as 250 J/cm², such as 300 J/cm², such as 350 J/cm², such as 400 J/cm², such as 450 J/cm².

12. The method according to any one of the preceding items, wherein the polymer hydrogel is prepared from chemicals selected from the group consisting of acrylics, methacrylics, acrylamides, and styrenics.

13. The method according to any one of the preceding items, wherein the polymer hydrogel reagents comprise a polymer/monomer selected from the group consisting of polyacrylamide (PA), polyethylene glycol (PEG), polyethylene glycol diacrylate (PEGDA), dimethacrylated PEG (PEGDM), Poly(ethylene glycol)-block-polylactide methyl ether (PEG-b-PLA), Poly-D,L-lactic acid/polyethylene glycol/poly-D,L-lactic acid (PDLLA-PEG), Poly(vinyl alcohol) (PVA), Chondroitin sulfate, Chitosan, Heparin, Dextran, Agarose, Alginate, Starch, Pectin, Polyvinylamine, Polyphosphazene, Poly(L-glutamic-acid), poly(N,N-dimethylacrylamide-co-furfuryl methacrylate), carboxymethylcellulose, poly(N-isopropylacrylamide), poly(aspartic acid), gellan gum, copoly(acrylamide), hydroxypropylmethylcellulose, collagen-inspired undecapeptide, collagen, fibrin, gelatin, silk fibroin, silk-MA, hyaluronic acid, methacrylated hyaluronic acid, methacrylated gelatin, methacrylated alginate, methacrylated collagen, and DNA.

14. The method according to any one of the preceding items, wherein the polymer hydrogel reagents comprise polyethylene glycol diacrylate (PEGDA) with a molecular weight between 0.5-100K, such as 3.4 K, such as 5 K, such as 10 K, such as 15 K, such as 20 K, such as 25 K.

15. The method according to any one of the preceding items, wherein the pore size of the substantially gelled parts is between 0.1 nm-1000 nm, such as 0.5 nm, such as 1 nm, such as 5 nm, such as 10 nm, such as 15 nm, such as 16 nm, such as 17 nm, such as 18 nm, such as 19 nm, such as 20 nm, such as 21 nm, such as 22 nm, such as 23 nm, such as 24 nm, such as 25 nm, such as 30 nm, such as 40 nm, such as 50 nm, such as 100 nm, such as 200 nm, such as 300 nm, such as 400 nm, such as 500 nm, such as 700 nm, such as 900 nm.

16. The method according to any one of the preceding items, wherein the pore size of the substantially gelled parts is less than 0.1 nm.

17. The method according to any one of the preceding items, wherein the concentration of monomer/polymer in the mixture is between 0.1%-40% (w/v), such as 0.5% (w/v), such as 0.75% (w/v), such as 1% (w/v), such as 2% (w/v), such as 3% (w/v), such as 4% (w/v), such as 5% (w/v), such as 6% (w/v), such as 7% (w/v), such as 8% (w/v), such as 9% (w/v), such as 10% (w/v), such as 11% (w/v), such as 13% (w/v), such as 15% (w/v), such as 17% (w/v), such as 19% (w/v) such as 20% (w/v), such as 25% (w/v), such as 30% (w/v), such as 35% (w/v).

18. The method according to any one of the preceding items, wherein the area of the substantially liquid compartments is between 50-5000μ², such as 100μ², such as 200μ², such as 300μ², such as 400μ², such as 500μ², such as 600μ², such as 700μ², such as 800μ², such as 900μ², such as 1000μ², such as 2000μ², such as 3000μ², such as 4000μ².

19. The method according to any one of the preceding items, wherein the thickness of the substantially gelled wall part of the compartment is between 1-5000 μm, such as 2 μm, such as 5 μm, such as 10 μm, such as 20 μm, such as 30 μm, such as 40 μm, such as 50 μm, such as 75 μm, such as 100 μm, such as 200 μm, such as 400 μm, such as 600 μm, such as 800 μm.

20. The method according to any one of the preceding items, wherein the height of the substantially liquid compartments is between 10-10000 μm, such as 20 μm, such as 50 μm, such as 100 μm, such as 200 μm, such as 300 μm, such as 400 μm, such as 500 μm, such as 750 μm, such as 1000 μm, such as 2000 μm, such as 4000 μm, such as 6000 μm, such as 8000 μm.

21. The method according to any one of the preceding items, wherein the volume of the substantially liquid compartments is between 500 μm³-0.5 mm³, such as 750 μm³, such as 1000 μm³, such as 1500 μm³, such as 2500 μm³, such as 5000 μm³, such as 7500 μm³, such as 10000 μm³, such as 50000 μm³, such as 100000 μm³, such as 500000 μm³, such as 0.001 mm³, such as 0.0025 mm³, such as 0.005 mm³, such as 0.0075 mm³, such as 0.01 mm³, such as 0.025 mm³, such as 0.05 mm³, such as 0.075 mm³, such as 0.1 mm³, such as 0.25 mm³.

22. The method according to any one of the preceding items, wherein the substantially gelled parts are cross-linked to limit diffusion of labelling reagents.

23. The method according to any one of items 2-22, wherein substantially no reaction product is formed in the substantially gelled parts.

24. The method according to any one of the preceding items, wherein the hydrogel contains two or more areas with different properties, such as different pore size, density, degree of solidity and/or compartment height.

25. The method according to any one of the preceding items, wherein the photoinitiator is selected from the group consisting of lithium phenyl-2,4,6-trimethylbenzoylphosphinate (LAP), Irgacure 651, Irgacure 184, Irgacure 907, Irgacure 2959 (12959), Diphenyl(2,4,6-trimethylbenzoyl)phosphine oxide (TPO), phenylbis (2,4,6-trimethylbenzoyl) phosphine oxide (BAPO), BAPO-OLi, BAPO-ONa, VA-086, Eosin Y, Riboflavin, Camphorquinone, 1,4-bis(4-(N,N-bis(6-(N,N,N-trimethylammonium)hexyl)amino)-styryl)-2,5-dimethoxybenzene tetraiodide (WSPI), 2,5-bis-[4-(diethylamino)-benzylidene]-cyclopentanone (BDEA), 3,3′-((((1E,1′E)-(2-oxocyclopentane-1,3-diylidene)bis(methanylylidene))bis(4,1 phenylene))bis(methylazanediyl))dipropanoate (P2CK), and ruthenium.

26. The method according to any one of the preceding items, wherein the photoinitiator is lithium phenyl-2,4,6-trimethylbenzoylphosphinate (LAP) or Irgacure 2959 (12959).

27. The method according to any one of the preceding items, wherein an agent is added to the mixture in order to make the hydrogel positively charged, optionally wherein the agent is a positively charged polymer, such as poly-lysine or polyethylenimine, further optionally wherein the agent is a positively charged monomer, such as 2-(methacryloyloxy)ethyl-trimethylammonium chloride (MAETAC), butyl methacrylate or [2-(acryloyloxy)ethyl]trimethylammonium chloride (AETAC).

28. The method according to any one of items 1-26, wherein an agent is added to the mixture in order to make the hydrogel negatively charged, optionally wherein the agent is a negatively charged polymer, such as DNA, polystyrene sulfonate and polyacrylic acid, further optionally wherein the agent is a negatively charged monomer, such as sodium 2-sulfoethyl methacrylate (SEMA).

29. The method according to any one of the preceding items, wherein the hydrogel is incubated at a temperature between 20° C. to 80° C., such as 25° C., such as 30° C., such as 35° C., such as 37° C., such as 40° C., such as 45° C., such as 50° C., such as 55° C., such as 60° C., such as 65° C., such as 70° C., such as 75° C.

30. The method according to any one of the preceding items, wherein the concentration of photoinitiator in the mixture is between 0.0001%-5% w/w, such as 0.0005% w/w, such as 0.001% w/w, such as 0.005% w/w, such as 0.01% w/w, such as 0.05% w/w, such as 0.1% w/w, such as 0.2% w/w, such as 0.3% w/w, such as 0.4% w/w, such as 0.5% w/w, such as 0.6% w/w, such as 0.07% w/w, such as 0.08% w/w, such as 0.9% w/w, such as 1% w/w, such as 1.5% w/w, such as 2% w/w, such as 2.5% w/w, such as 3% w/w, such as 3.5% w/w, such as 4% w/w, such as 4.5% w/w.

31. The method according to any one of items 2-30, wherein the target is a nucleic acid and the reaction product is a nucleic acid amplified from said target nucleic acid.

32. The method according to any one of items 2-31, wherein the labelling reagents comprise reagents for isothermal amplification, such as reagents for rolling circle amplification (RCA), loop mediated isothermal amplification (LAMP), multiple displacement amplification (MDA), nucleic acid sequence-based amplification (NASBA), helicase-dependent amplification (HDA), recombinase polymerase amplification (RPA), whole genome amplification (WGA), self-sustained sequence replication (3SR), transcription mediated amplification (TMA), signal-mediated amplification of RNA technology (SMART), ramification amplification (RMA), proximity ligation assay (PLA) and/or strand displacement amplification (SDA).

33. The method according to any one of items 2-32, wherein the labelling reagents further comprise dimethyl sulfoxide (DMSO), Propyl sulfoxide, Methyl sec-butyl sulfoxide, Tetramethylene Sulfoxide, Methyl sulfone, Sulfolane, single-stranded DNA binding protein (SSB), TaqSSB, T4 gene 32 protein (gp32), PEG4000, PEG400, Non-ionic detergents (such as, Tween-20, Triton X-100, NP-40), Glycerol, Propylene glycol, Ethylene glycol, Betaine, Trehalose, Formamide, Acetamide, 2-pyrrolidone, Tetramethyl ammonium chloride (TMAC), TMA oxalate, TMA hydrogen sulfate, TEA-CI, TPA-CI, TPA-acetate, TBA-CI, 7-deaza-2′-deoxyguanosine, Magnesium, Dithiothreitol (DTT), pyrophosphatase and/or bovine serum albumin (BSA).

34. The method according to any one of items 32-33, wherein the isothermal amplification reagents further comprise magnesium pyrophosphate, calcium pyrophosphate, hydroxynaphthol blue or calcein.

35. The method according to item 34, wherein magnesium or calcium pyrophosphate is deposited in the vicinity of said biological particle as a result of the isothermal amplification reaction.

36. The method according to any one of items 32-35, wherein the isothermal amplification reagents further comprise a dye to label the amplified nucleic acid.

37. The method according to any one of items 32-36, wherein the dye is linked to a primer or to deoxynucleotide triphosphates (dNTPs) which are incorporated during the isothermal amplification.

38. The method according to any one of items 36-37, wherein the dye is an intercalating dye or a fluorescent tagged probe.

39. The method according to item 38, wherein the intercalating dye is SYBR Green, SYBR Gold, LAMP Dye, DAPI, Hoechst, ToPro3, Draq5, Draq7, RedDot1, RedDot2, Propidium iodide, Ethidium bromide and/or Evagreen.

40. The method according to item 38, wherein the fluorescent probe is FITC, FAM, PE, APC, Cy3, Cy5, Cy7, PerCP, AF488, AF647, AF555, CF488 CF555, CF647, any of the fluorescent probes from the Alexa fluorophore family, any of the fluorescent probes from the Biotium fluorophore family, any of the fluorescent probes from the Atto fluorophore family, quantum dots, and/or a polymer dye from the Brilliant violet dye family.

41. The method according to any one of items 32-40, wherein the multiple displacement amplification (MDA) or loop mediated isothermal amplification (LAMP) reagents comprise a primer-dye and a primer-quencher set.

42. The method according to any one of the preceding items, wherein the particle is a biological cell, a viral particle, a bacteriophage, a bead, an exosome, a plasmid, an extrachromosomal DNA, or an extrachromosomal circular DNA.

43. The method according to any one of items 2-42, wherein the target is a nucleic acid in a virus, a viral particle, a bacteriophage, a bacterium, a protozoan, a yeast, a fungus, a plant cell, an insect cell, a mammalian cell, an exosome, a plasmid, an extrachromosomal DNA (ecDNA), an extrachromosomal circular DNA (eccDNA), an aptamer, a nucleic acid tag linked to an aptamer, a molecular beacon, a nucleic acid tag linked to an antibody or a receptor ligand, or residual nucleic acid in pharmaceutical products.

44. The method according to item 43, wherein the nucleic acid is DNA or RNA, such as viral RNA or mRNA.

45. The method according to item 43, wherein the mammalian cell is a cell from a non-human cell line.

46. The method according to item 43, wherein the mammalian cell is a cell from a human.

47. The method according to any one of items 43 to 46, wherein the nucleic acid is a medical marker of a disease, such as an oncogene or a marker of a genetic disease or an autoimmune disease.

48. The method according to any one of items 2-41, wherein the target is a protein, such as an enzyme, and the method comprises adding a labelling reagent capable of binding said protein, the labelling reagent comprising a nucleic acid tag, such as a DNA tag, and/or an antibody.

49. The method according to any one of items 2-48, wherein the reaction product is a fluorescent molecule, or where a fluorescent molecule can bind to the reaction product.

50. The method according to any one of items 2-49, wherein the reaction product is colored.

51. The method according to any one of items 2-50, wherein the target is an enzyme and the labelling reagents comprise a reagent capable of reacting with said enzyme, the reagent comprising an enzyme substrate and/or enzyme co-factors.

52. The method according to any one of items 2-51, wherein the enzyme is selected from the group consisting of phosphatases such as alkaline phosphatase, β-galactosidase, β-glucuronidase, β-glucose-6-phosphate dehydrogenase, glucose oxidase, urease, luciferase, β-lactamase, β-amylase, and peroxidase, such as for example horseradish peroxidase.

53. The method according to any one of items 2-52, wherein the labelling reagents comprise a targeting moiety, such as an antibody, linked to an enzyme, wherein said targeting moiety is capable reacting with and/or binding the target and/or the biological particle.

54. The method according to any one of the preceding items, wherein the sample comprises viruses, viral particles, bacteriophages, bacteria, protozoa, yeast, fungi, plant cells, insect cells, mammalian cells, exosomes, proteins, enzymes, plasmids, DNA, RNA, mRNA, microRNA, extrachromosomal DNA (ecDNA), extrachromosomal circular DNA (eccDNA), and/or residual nucleic acid in pharmaceutical products.

55. The method according to item 54, wherein the mammalian cells are from a non-human cell line.

56. The method according to item 54, wherein the mammalian cells are human cells.

57. The method according to any one of the preceding items, wherein the sample comprises viruses, bacteria, yeast, mammalian cells, plasmids, exosomes, extrachromosomal DNA (ecDNA), extrachromosomal circular DNA (eccDNA), DNA, RNA, mRNA, microRNA and/or residual DNA in pharmaceutical products.

58. The method according to any one of the preceding items, wherein the sample is a body fluid sample, a tissue sample, a fermentation sample, a liquid cultivation sample, a cell culture sample, a water sample, a beverage sample, a pharmaceutical sample, an environmental sample, a sewage sample, a diagnostic sample or a sample of a pharmaceutical product.

59. The method according to item 58, wherein the body fluid sample is selected from the group consisting of samples from swabs, blood, plasma, serum, urine, bile, saliva, semen, cerebrospinal fluid and mucus.

60. The method according to item 58, wherein the tissue sample is selected from the group consisting of tissue samples from liver, kidney, muscle, brain, lung, skin, thymus, spleen, gastrointestinal tract, pancreas and thyroid gland.

61. The method according to any one of items 57-60, wherein the sample is a sample from a human.

62. The method according to and one of items 1-41 and 48-61, wherein the biological particle is a cell, and the method further comprises isolating cells comprising the target from the substantially liquid compartments.

63. The method according to item 62, wherein said cells are selected from the group consisting of bacteria, yeast, protozoa, fungus, plant cells, mammalian cells and insect cells, and the target is selected from the group consisting of a nucleic acid, an exosome, a plasmid, an extrachromosomal DNA (ecDNA), an extrachromosomal circular DNA (eccDNA), an aptamer, a nucleic acid tag linked to an aptamer, a molecular beacon, a nucleic acid tag linked to an antibody or a receptor ligand, an antibody, a protein and an enzyme.

64. The method according to any one of items 62-63, wherein said cells are isolated using a robotics platform.

65. The method according to any one of items 2-64, further comprising quantifying the amount, such as the concentration, of biological particles and/or target in the sample, by counting the number of substantially liquid compartments with reaction product.

66. The method according to item 65, wherein the number of substantially liquid compartments with reaction product are counted in an image using image analysis to identify the number of substantially liquid compartments with reaction product.

67. The method according to any one of items 4-66, further comprising recording an image of the hydrogel or mixture before incubation and/or induction of hydrogel formation.

68. The method according to item 67, wherein said image is used to determine the background of the hydrogel or mixture before incubation.

69. The method according to any one of items 4-68, wherein multiple images of the hydrogel are recorded while the hydrogel is being incubated, such as wherein images are compared following image processing to determine the number of substantially liquid compartments with reaction product.

70. The method of item 69, wherein the image processing determines the amount of reaction product in each substantially liquid compartment, optionally wherein said amount is correlated to the discrete number of biological particles in each substantially liquid compartment.

71. The method according to item 69, wherein the multiple images are used to evaluate the formation of reaction product over time.

72. The method according to any one of items 66-71, wherein the recording of images comprises the use of a confocal scanner, a microscope, a fluorescence microscope, an image cytometer, and/or an automated particle counter.

73. The method according to any one of items 66-72, wherein the recording of images comprises the use of one or more optical channels.

74. The method according to any one of items 66-73, wherein the recording of images comprises the use of one or more fluorescent channels.

75. The method according to any one of items 66-74, wherein the images are recorded using an array of detection devices.

76. The method according to any one of items 66-75, wherein the images are recorded using a two-dimensional array of detection devices.

77. The method according to any one of items 66-76, wherein the images are 3D images.

78. The method according to any one of items 66-77, wherein the images are recorded using a CCD, a CMOS, a video camera, photomultiplier tubes, photodiode, avalanche photodiode (APD) or a photon counting camera.

79. The method according to any one of items 66-78, wherein the recorded image is processed.

80. The method according to item 79, wherein the recorded image is processed using data processing means.

81. The method according to any one of items 79-80, wherein the image is processed using artificial intelligence.

82. The method according to any one of items 80-81, wherein the data processing means determining or estimating the number of biological particles located in an individual substantially liquid compartment.

83. The method according to item 82, wherein the determination or estimation of the number of biological particles located in an individual compartment is used to determine the number of targets and/or biological particles in the sample.

84. The method according to any one of items 80-83, wherein the data processing improves the detection and/or the specificity of the method.

85. The method according to any one of the preceding items, wherein sample is loaded into a sample compartment, such as wherein the mixture is cured inside said sample compartment.

86. The method according to item 85, wherein the sample compartment forms part of a cassette.

87. The method according to any one of items 85-86, wherein the sample compartment is defined by two parallel sheets of transparent material, preferably plastic material or glass material with a given distance between them.

88. The method according to any one of items 85-87, wherein the cassette comprises channels for loading the sample.

89. The method according to any one of items 85-88, wherein the cassette comprises channels for flowing the sample within the cassette.

90. The method according to any one of items 85-89, wherein the cassette comprises a valve for regulating the flow of sample.

91. The method according to any one of items 85-90, wherein at least one wall of the sample compartment is transparent.

92. The method according to any one of items 85-91, wherein the cassette is removable.

93. The method according to any one of items 85-92, wherein the cassette is single-use and/or disposable.

94. The method according to any one of items 85-93, wherein the photoinitiator is preloaded into the sample compartment or into a channel part of the cassette.

95. The method according to any one of the preceding items, wherein a cellular stain is added to the mixture prior to curing the hydrogel.

96. The method according to item 95, wherein the cellular stain is a stain which stains the whole cell or a part of the cell, such as the cell membrane, the cell wall, the cytoplasm, the nucleus, the mitochondria and/or nucleic acids.

97. The method according to any one of the preceding items, further comprising illuminating the hydrogel, whereby cells comprised in the sample are lysed.

98. The method according to any one of the preceding items, wherein a lysing agent is added to the mixture prior to curing the hydrogel.

99. The method according to any one of the preceding items, wherein the sample is pre-treated using a method comprising one or more of the following steps:
a. incubating the sample at 98° C. for 5 minutes; and/or
b. mixing the sample with 96% ethanol; and/or
c. mixing the sample with 1-1000 mM sodium hydroxide; and/or
d. mixing the sample with detergent, such as NP40, tween20 or triton X100; and/or
e. mixing the sample with saponin; and/or
f. mixing the sample with 96% methanol.

100. A method of creating compartments in a sample with biological particles, said method comprising the steps of:
a. mixing the sample with curable polymer hydrogel reagents, the curable polymer hydrogel reagents comprising a photoinitiator;
b. illuminating the sample with a light pattern to induce gelling in illuminated parts thereby creating a hydrogel with a number of substantially liquid compartments separated by substantially gelled parts.

101. A kit for detecting a biological particle in a sample, said kit comprising:
a. polymer hydrogel reagents; and
b. additional reagents, the reagents being capable of generating a reaction product in the vicinity of particles having a specific target.

102. The kit according to item 101, wherein the additional reagents comprise reagents as defined in any one of items 2-3, 32-41, 48 and 51-52, and wherein the hydrogel reagents are as defined in any one of items 12-14, 17, 25-28, and 30.

103. The kit according to any one of items 101-102, further comprising a cellular stain.

104. The kit according to any one of items 101-103, further comprising a lysing agent.

105. A system for detecting a biological particle in a sample, said system comprising:
f. a holder for a cassette with a sample compartment,
g. image forming means capable of forming an image of at least part of the sample in the sample compartment on image acquisition means,
h. image processing means,
i. at least one illumination source capable of illuminating the sample in the sample compartment with a light pattern to induce gelling in illuminated parts thereby creating a hydrogel with a number of substantially liquid compartments separated by substantially gelled parts; and
j. thermostatically controlled heating means capable of heating the sample in the sample compartment.

106. The system according to item 105, wherein at least one illumination source is capable of providing specific energy of at least 0.001 J/cm², such as at least 0.01 J/cm², such as at least 0.1 J/cm², such as at least 0.2 J/cm², such as at least 0.25 J/cm², such as at least 0.3 J/cm², such as at least 0.5 J/cm², such as at least 0.75 J/cm², such as at least 0.1 J/cm², such as at least 0.5 J/cm², such as at least 1 J/cm², such as at least 5 J/cm², such as at least 10 J/cm², such as at least 15 J/cm², such as at least 20 J/cm², such as at least 25 J/cm², such as at least 30 J/cm², such as at least 50 J/cm², such as at least 75 J/cm², such as at least 100 J/cm², such as at least 150 J/cm², such as at least 200 J/cm², such as at least 250 J/cm², such as at least 300 J/cm², such as at least 350 J/cm², such as at least 400 J/cm², such as at least 450 J/cm².

107. The system according to any one of items 105-106, wherein the illumination source is capable of providing electromagnetic radiation with a wavelength of at least 100 nm, such as at least 150 nm, such as at least 200 nm, such as at least 250 nm, such as at least 270 nm, such as at least 300 nm, such as at least 350 nm, such as at least 365 nm, such as at least 375 nm, such as at least 380 nm, such as at least 385 nm, such as at least 390 nm, such as at least 395 nm, such as at least 400 nm, such as at least 444 nm, such as at least 450 nm, such as at least 500 nm, such as at least 524 nm, such as at least 550 nm, such as at least 600 nm, such as at least 650 nm, such as at least 700 nm, such as at least 750 nm, such as at least 800 nm, such as at least 850 nm, such as at least 900 nm, such as at least 950 nm.

108. The system according to any one of items 105-107, wherein one illumination source is capable of inducing hydrogel formation, and at least one other illumination source is capable of illuminating the sample to generate an image of at least part of the sample on the image acquisition means.

109. The system according to any one of items 105-108, wherein the system comprises at least two illumination sources, such as at least 3, 4, 5, 6, 7, or 8 illumination sources, such as excitation light sources, bright field light sources, such as for example a UV light source.

110. The system according to any one of items 105-109, wherein the image forming means is capable of forming a bright field image, a dark field image, and/or a phase contrast image.

111. The system according to any one of items 105-110, wherein the heating means comprise a heating device configured for heating the sample to a temperature of 40-70° C.

112. The system according to any one of items 105-111, wherein the biological particle is a cell, and the system further comprises means for identifying and isolating cells comprising the target from the substantially liquid compartments.

113. The system according to item 112, wherein said cells are selected from the group consisting of bacteria, yeast, protozoa, fungus, plant cells, mammalian cells and insect cells, and the target is selected from the group consisting of a nucleic acid, an exosome, a plasmid, an extrachromosomal DNA (ecDNA), an extrachromosomal circular DNA (eccDNA), an aptamer, a nucleic acid tag linked to an aptamer, a molecular beacon, a nucleic acid tag linked to an antibody or a receptor ligand, an antibody, a protein and an enzyme.

114. The system according to any one of items 112-113, wherein the system comprises a robotic platform for isolating cells from the substantially liquid compartments.

1. A method of detecting and/or analyzing a biological particle in a sample, said method comprising the steps of:
a. mixing the sample with polymer hydrogel reagents and nucleic acid amplification reagents, the nucleic acid amplification reagents being capable of generating a reaction product in the vicinity of particles having a specific target;
b. inducing curing of the mixture to a hydrogel;
c. incubating the mixture or hydrogel, whereby one or more reaction products are produced as a result of the nucleic acid amplification reaction;
d. recording one or more images of the hydrogel.

2. The method according to claim 1, wherein steps b-d can be conducted in any order, preferably wherein step b is conducted before step c.

3. The method according to any one of the preceding claims, wherein the reagents comprise a primer set and optionally an aptamer, a molecular beacon, an antibody, a probe, or a receptor ligand with a nucleotide tag.

4. The method according to any one of the preceding claims, wherein;
i. the polymer hydrogel reagents further comprise a photoinitiator;
ii. the mixture is cured to a hydrogel by illuminating the sample with a light pattern to induce gelling in illuminated parts thereby creating a number of substantially liquid compartments separated by substantially gelled parts; and
iii. one or more images of the hydrogel are recorded thereby detecting or analyzing biological particles in the substantially liquid compartments.

5. The method according to claim 4, wherein each substantially liquid compartment comprises a discrete number of biological particles, such as 0, 1, 2, 3, 4, 5, 6, 7, 8, 9 or 10 particles, preferably 0, 1, 2, or 3 particles.

6. The method according to any one of the preceding claims, wherein the pattern is created using UV photolithography, wherein a collimated light source illuminates the sample through a photolithographic mask.

7. The method according to any one of the preceding claims, wherein the pattern is created by inserting a mask in the light path between the light source and the sample, or wherein the pattern is created by illuminating the sample with a laser, preferably whereby a predetermined pattern is projected across at least part of the sample.

8. The method according to any one of the preceding claims, wherein:
i. the sample comprises RNA, extrachromosomal DNA (ecDNA) and/or extrachromosomal circular DNA (eccDNA), the isothermal amplification reagents being capable of generating a reaction product in the vicinity of the RNA, ecDNA and/or eccDNA;
ii. the mixture or hydrogel is incubated, whereby one or more amplicons are produced as a result of the isothermal amplification reaction amplifying whole or part of the RNA, ecDNA and/or eccDNA, if said RNA, ecDNA and/or eccDNA is present in the sample.

9. The method according to claim 8, wherein the extrachromosomal DNA is or contains a marker of cancer, such as an oncogene or a marker for a genetic disease or an autoimmune disease.

10. The method according claim 8, wherein the extrachromosomal DNA is a plasmid.

11. The method according to any one of the preceding claims, wherein:
i. the sample comprises viral particles, the nucleic acid amplification reagents being capable of generating a reaction product in the vicinity of the viral particles;
ii. the mixture or hydrogel is incubated, whereby one or more amplicons are produced as a result of the nucleic acid amplification reaction amplifying whole or part of the nucleic acids present in the viral particle, if said viral particle is present in the sample.

12. The method according to any one of the preceding claims, wherein a cellular stain is mixed with the reagents prior to curing the hydrogel.

13. The method according to any one of the preceding claims, wherein curing is induced by a change in temperature, application of a current, by radiation, or by pressure.

14. The method according to any one of the preceding claims, wherein curing is done in more than one step, such as in two steps.

15. The method according to any one of the preceding claims, wherein the duration of the step of curing the mixture to a hydrogel is less than 30 minutes, preferably less than 15 minutes, preferably less than 10 minutes, preferably less than 8 minutes, preferably less than 6 minutes, preferably less than 5 minutes, preferably less than 2 minutes, preferably less than 1 min, preferably less than 30 seconds.

16. The method according to any one of the preceding claims, wherein the polymer hydrogel is prepared from chemicals selected from the group consisting of acrylics, methacrylics, acrylamides, styrenics, norbornenes and thiols.

17. The method according to any one of the preceding claims, wherein the polymer hydrogel reagents comprise a polymer/monomer selected from the group consisting of polyacrylamide (PA), polyethylene glycol (PEG), polyethylene glycol diacrylate (PEGDA), dimethacrylated PEG (PEGDM), Poly(ethylene glycol)-block-polylactide methyl ether (PEG-b-PLA), polyethylene glycol thiols (PEG-SH), polyethylene glycols norbornene-terminated (PEG-norbornene), Poly-D,L-lactic acid/polyethylene glycol/poly-D,L-lactic acid (PDLLA-PEG), Poly(vinyl alcohol) (PVA), Chondroitin sulfate, Chitosan, Heparin, Dextran, Agarose, Alginate, Starch, Pectin, Polyvinylamine, Polyphosphazene, Poly(L-glutamic-acid), poly(N,N-dimethylacrylamide-co-furfuryl methacrylate), carboxymethylcellulose, poly(N-isopropylacrylamide), poly(aspartic acid), gellan gum, copoly(acrylamide), hydroxypropylmethylcellulose, collagen-inspired undecapeptide, collagen, fibrin, gelatin, silk fibroin, silk-MA(glycidyl-methacrylate-modified silk fibroin), hyaluronic acid, methacrylated hyaluronic acid, methacrylated gelatin, methacrylated alginate, methacrylated collagen, methacrylated peptide, peptide and DNA.

18. The method according to any one of the preceding claims, wherein the polymer hydrogel reagents comprise polyethylene glycol diacrylate (PEGDA) with a molecular weight between 0.5-100 K, such as 1-80 K, for example 1.5-60 K, such as 2-40 K, for example 2.5-35 K, such as 3-30 K, for example 3-25 K, such as 3-20 K, for example 3-10 K, such as 3-7 K, for example 3-5 K, such as 3.3-4 K.

19. The method according to any one of the preceding claims, wherein the pore size of the hydrogel is in the range between 1 nm-1,000 nm, such as 1 nm-900 nm, for example 1-700 nm, such as 5-500 nm, such as 10-400 nm, for example 15-300 nm, such as 16-200 nm, for example 17 nm-100 nm, such as 18-50 nm, such as 19-40 nm, such as 20-30 nm, such as 21-25 nm.

20. The method according to any one of the preceding claims, wherein the concentration of monomer/polymer in the mixture is between 0.1%-40% (w/v), such as 1-25, for example 2-10, such as 5-10

21. The method according to any one of the preceding claims, wherein the substantially gelled parts are cross-linked preferably to limit diffusion of reagents such as labelling reagents.

22. The method according to any one of the preceding claims, wherein substantially no reaction product is formed in the substantially gelled parts.

23. The method according to any one of the preceding claims, wherein the hydrogel is heterogeneous.

24. The method according to any one of the preceding claims, wherein the hydrogel contains two or more areas with different properties, such as areas with different pore size, density, compartment height and/or different degree of solidity.

25. The method according to any one of the preceding claims, wherein the hydrogel contains two or more areas with different properties, such as areas of high degree of solidity and areas of substantially no solidity.

26. The method according to any one of the preceding claims, wherein a pattern is made in the hydrogel, such as a pattern which generates multiple compartments.

27. The method according to any one of the preceding claims, wherein the compartments are placed in rows in the hydrogel.

28. The method according to any one of the preceding claims, wherein the area of the compartments is in the range 50-40,000 μm², for example 100-35.000 μm², such as 200-32,000 μm², for example 300-33,000 μm², such as 400-32,500 μm², for example 450-32,000 μm², such as 475-31,500 μm², for example 490-31,415 μm².

29. The method according to any one of the preceding claims, wherein the thickness of the wall of the compartment such as the substantially gelled wall part of the compartment is in the range between 1-5,000 μm, such as 10-2,500 μm, for example 20-1,000 μm, such as 30-500 μm, for example 40-250 μm, such as 45-250 μm, such as 50-200 μm, such as 75-150 μm.

30. The method according to any one of the preceding claims, wherein the height of the compartments such as the substantially liquid compartments is between 10-10,000 μm, for example 50-1,000 μm, such as 50-500 μm, for example 75-200 μm, such as 90-150 μm.

31. The method according to any one of the preceding claims, wherein the volume of the compartments such as the substantially liquid compartments is between 100,000 μm³-40,000,000 μm³, for example 250,000 μm³-35,000,000 μm³, such as 400,000 μm³-32,500,000 μm³, for example 450,000 μm³-32,000,000 μm³, such as 490,000-31,415,000 μm³.

32. The method according to any one of the preceding claims, wherein an agent is added to the mixture in order to make the hydrogel positively charged, optionally wherein the agent is a positively charged polymer, such as poly-lysine or polyethylenimine, further optionally wherein the agent is a positively charged monomer, such as 2-(methacryloyloxy)ethyl-trimethylammonium chloride (MAETAC), butyl methacrylate or [2-(acryloyloxy)ethyl]trimethylammonium chloride (AETAC).

33. The method according to any one of the preceding claims, wherein the agent is a positively charged polymer, such as poly-lysine or polyethylenimine.

34. The method according to any one of the preceding claims, wherein the agent is a positively charged monomer, such as 2-(methacryloyloxy)ethyl-trimethylammonium chloride (MAETAC), butyl methacrylate or [2-(acryloyloxy)ethyl]trimethylammonium chloride (AETAC).

35. The method according to any one of the preceding claims, wherein an agent is added to the mixture in order to make the hydrogel negatively charged, optionally wherein the agent is a negatively charged polymer, such as DNA, polystyrene sulfonate and polyacrylic acid, further optionally wherein the agent is a negatively charged monomer, such as sodium 2-sulfoethyl methacrylate (SEMA).

36. The method according to any one of the preceding claims, wherein the agent is a negatively charged polymer, such as DNA, polystyrene sulfonate and polyacrylic acid.

37. The method according to any one of the preceding claims, wherein the agent is a negatively charged monomer, such as sodium 2-sulfoethyl methacrylate (SEMA).

38. The method according to any one of the preceding claims, wherein the hydrogel is incubated at a temperature between 20° C. to 80° C., such as 25° C.-75° C., such as 30° C.-70° C., such as 35° C.-70° C., such as 37° C.-70° C., such as 40° C.-70° C., such as 45° C.-70° C., such as 50° C.-70° C., such as 55° C.-70° C., such as 55° C.-67° C., such as 60° C.-65° C.

39. The method according to any one of the preceding claims, wherein the polymer hydrogel reagents comprise a photoinitiator.

40. The method according to any one of the preceding claims, wherein the photoinitiator is selected from the group consisting of lithium phenyl-2,4,6-trimethylbenzoylphosphinate (LAP), Irgacure 651, Irgacure 184, Irgacure 907, Irgacure 2959 (12959), Diphenyl(2,4,6-trimethylbenzoyl)phosphine oxide (TPO), phenylbis (2,4,6-trimethylbenzoyl) phosphine oxide (BAPO), BAPO-OLi, BAPO-ONa, VA-086, Eosin Y, Riboflavin, Camphorquinone, 1,4-bis(4-(N,N-bis(6-(N,N,N-trimethylammonium)hexyl)amino)-styryl)-2,5-dimethoxybenzene tetraiodide (WSPI), 2,5-bis-[4-(diethylamino)-benzylidene]-cyclopentanone (BDEA), 3,3′-((((1E,1′E)-(2-oxocyclopentane-1,3-diylidene)bis(methanylylidene))bis(4,1 phenylene))bis(methylazanediyl))dipropanoate (P2CK), and ruthenium.

41. The method according to any one of the preceding claims, wherein the photoinitiator is lithium phenyl-2,4,6-trimethylbenzoylphosphinate (LAP) or Irgacure 2959 (I2959).

42. The method according to any one of the preceding claims, wherein the concentration of photoinitiator in the mixture is between 0.0001%-5% w/w, such as 0.001 to 2.5%, for example 0.01 to 1%.

43. The method according to any one of the preceding claims, wherein the mixture is cured to a hydrogel at a wavelength between 100 nm-1,200 nm, for example 100 nm-1,000 nm, such as 150 nm-850 nm, for example 200 nm-800 nm, such as 300 nm-750 nm, for example 350 nm-700 nm, such as 350 nm-600 nm, for example 375 nm-500 nm, such as 390 nm-400 nm.

44. The method according to any one of the preceding claims, wherein the mixture is cured to a hydrogel at an intensity of 0.001 J/cm²-500 J/cm², such as 0.01 to 400 J/cm², for example 0.1 to 300 J/cm², such as 1 to 100 J/cm², for example 10-50 J/cm².

45. The method according to any one of the preceding claims, wherein a current is used to cure the mixture to a hydrogel.

46. The method according to claim 45, wherein the hydrogel is cured with a current after being cured by any of the curing methods according to any one of the preceding claims.

47. The method according to claim 45, wherein curing the hydrogel with a current decreases the diffusion rates in the hydrogel.

48. The method according to any one of the preceding claims, wherein the polymer hydrogel reagents comprise an agent which enables the hydrogel to be cured by a change in temperature.

49. The method according to claim 48, wherein the agent is selected from the group consisting of bovine serum albumin (BSA), laminin, type IV collagen, heparan sulfate proteoglycan and/or entactin.

50. The method according to any one of claims 48-49, wherein the hydrogel is cured by increasing the temperature between 20° C.-50° C., such as 25° C., such as 30° C., such as 35° C., such as 40° C., such as 45° C.

51. The method according to any one of the preceding claims, wherein the biological particle comprises or is a target nucleic acid and the reaction product is a nucleic acid amplified from said target nucleic acid.

52. The method according to any one of the preceding claims, wherein the target is a nucleic acid and the reaction product is a nucleic acid amplified from said target nucleic acid.

53. The method according to claim 52, further comprising a step for pre-amplification of the target, said step comprising amplifying the target such as a nucleic acid, whereby more of said target, such as a higher amount, is produced, preferably said step is conducted before step c, such as before step b, such as before step a.

54. The method according to claim 53, wherein pre-amplification of the target comprises polymerase chain reaction (PCR), multiple displacement amplification (MDA), strand displacement amplification (SDA), recombinase polymerase amplification (RPA), multiple annealing and looping based amplification cycles (MALBAC) or primary template assisted amplification (PTA).

55. The method according to any one of the preceding claims, further comprising detection and/or differentiation of single nucleotide polymorphisms (SNPs) in a target nucleic acid, whereby at least one population of biological particles, such as at least two, three, four, five or more populations of biological particles can be detected and/or differentiated from each other.

56. The method according to claim 55, wherein:
a. the at least one population of biological particles comprises at least a wild-type allele or a mutant allele; or
b. the at least two populations of biological particles comprise at least a wild-type and at least one mutant allele, or two different mutant alleles.

57. The method according to claim 55-56, wherein the nucleic adid amplification reagents comprise or consist of reagents for loop-primer endonuclease cleavage-loop-mediated isothermal amplification (LEC-LAMP), PA-LAMP, RALA-LAMP or CHB-LAMP.

58. The method according to any one of the preceding claims, wherein the nucleic acid amplification reagents are isothermal amplification reagents comprising reagents for rolling circle amplification (RCA), loop mediated isothermal amplification (LAMP), multiple displacement amplification (MDA), nucleic acid sequence-based amplification (NASBA), helicase-dependent amplification (HDA), recombinase polymerase amplification (RPA), whole genome amplification (WGA), self-sustained sequence replication (3SR), transcription mediated amplification (TMA), signal-mediated amplification of RNA technology (SMART), ramification amplification (RMA), proximity ligation assay (PLA) and/or strand displacement amplification (SDA).

59. The method according to any one of the preceding claims, wherein the isothermal amplification reagents comprise
a. a DNA polymerase, such as Phi29 or mutant versions thereof such as EquiPhi29 or QualiPhi; BST or mutant versions thereof such as large fragment, BST 2.0, BST 3.0 or IsoPol BST; BSU; BSM; IsoPol SD+; and/or Klenow; and
b. dNTPs; and optionally,
c. a reverse transcriptase.

60. The method according to any one of the preceding claims, wherein the nucleic acid amplification reagents comprise reagents for PCR, qPCR, or RT-qPCR.

61. The method according to any one of the preceding claims, wherein the reagents further comprise dimethyl sulfoxide (DMSO), Propyl sulfoxide, Methyl sec-butyl sulfoxide, Tetramethylene Sulfoxide, Methyl sulfone, Sulfolane, single-stranded DNA binding protein (SSB), TaqSSB, T4 gene 32 protein (gp32), RecA, Tth RecA, nicking endoncleases (e.g. Nt.BsmAl, Nt.BstNBI, Nb.BsrDI), PEG4000, PEG400, Non-ionic detergents (such as, Tween-20, Triton X-100, NP-40), Glycerol, Propylene glycol, Ethylene glycol, Betaine, Trehalose, Formamide, Acetamide, 2-pyrrolidone, Tetramethyl ammonium chloride (TMAC), TMA oxalate, TMA hydrogen sulfate, TEA-CI, TPA-CI, TPA-acetate, TBA-CI, 7-deaza-2′-deoxyguanosine, Magnesium, Dithiothreitol (DTT), pyrophosphatase and/or bovine serum albumin (BSA).

62. The method according to any one of the preceding claims, wherein the nucleic acid amplification reagents further comprise magnesium pyrophosphate, calcium pyrophosphate, hydroxynaphthol blue or calcein.

63. The method according to claim 62, wherein magnesium or calcium pyrophosphate is deposited in the vicinity of said biological particle as a result of the nucleic amplification reaction.

64. The method according to any one of the preceding claims, wherein the nucleic acid amplification reagents further comprise a dye to label the amplified nucleic acid.

65. The method according to any one of the preceding claims, wherein the dye is linked to a primer or to deoxynucleotide triphosphates (dNTPs) which are incorporated during the isothermal amplification.

66. The method according to any one of claims 64-65, wherein the dye is an intercalating dye or a fluorescent tagged probe.

67. The method according to claim 66 any one of the preceding claims, wherein the intercalating dye is SYBR Green, SYBR Gold, Syto 9, Syto 80, SYBR 14, Syto 41, Syto 60, Syto 62, Syto 64, LAMP Dye, DAPI, Hoechst, ToPro3, Draq5, Draq7, RedDot1, RedDot2, Propidium iodide, Ethidium bromide and/or Evagreen.

68. The method according to claim 66, wherein the fluorescent probe is FITC, FAM, PE, APC, Cy3, Cy5, Cy7, PerCP, AF488, AF647, AF555, CF488 CF555, CF647, any of the fluorescent probes from the Alexa fluorophore family, any of the fluorescent probes from the Biotium fluorophore family, any of the fluorescent probes from the Atto fluorophore family, such as Atto 488 and Atto 565, quantum dots, and/or a polymer dye from the Brilliant violet dye family.

69. The method according to any one of the preceding claims, wherein the multiple displacement amplification (MDA) or loop mediated isothermal amplification (LAMP) reagents comprise a primer-dye and a primer-quencher set.

70. The method according to any one of the preceding claims, wherein the particle is a biological cell, a viral particle, a bacteriophage, a bead, an exosome, a plasmid, RNA, an extrachromosomal DNA, or an extrachromosomal circular DNA.

71. The method according to claim 70, wherein the particle is a bead, such as a magnetic bead, optionally wherein the target is linked to the bead via affinity binding such as biotin-streptavidin/avidin or nucleic acid hybridisation,

72. The method according to any one of the preceding claims, wherein the target is a nucleic acid in a virus, a viral particle, a bacteriophage, a bacterium, a protozoan, a yeast, a fungus, a plant cell, an insect cell, a mammalian cell, an exosome, a plasmid, an extrachromosomal DNA (ecDNA), an extrachromosomal circular DNA (eccDNA), an aptamer, a nucleic acid tag linked to an aptamer, a molecular beacon, a nucleic acid tag linked to an antibody or a receptor ligand, or residual nucleic acid in pharmaceutical products.

73. The method according to claim 72, wherein the nucleic acid is DNA or RNA, such as viral RNA or mRNA.

74. The method according to claim 72, wherein the mammalian cell is a cell from a non-human cell line.

75. The method according to claim 72, wherein the mammalian cell is a cell from a human.

76. The method according to any one of the preceding claims, wherein the nucleic acid is a medical marker of a disease, such as an oncogene or a marker of a genetic disease or an autoimmune disease.

77. The method according to any one of the preceding claims, wherein the reaction product is a product that causes reflection, refraction, diffraction, interaction, scattering or absorbance of light emitted from a light source onto the sample.

78. The method according to any one of the preceding claims, wherein the reaction product is a fluorescent molecule, or where a fluorescent molecule can bind to the reaction product.

79. The method according to any one of the preceding claims, wherein the reaction product is colored.

80. The method according to any one of the preceding claims, wherein the sample comprises viruses, viral particles, bacteriophages, bacteria, protozoa, yeast, fungi, plant cells, insect cells, mammalian cells, exosomes, proteins, enzymes, plasmids, DNA, RNA, mRNA, microRNA, extrachromosomal DNA (ecDNA), extrachromosomal circular DNA (eccDNA), and/or residual nucleic acid in pharmaceutical products.

81. The method according to claim 80, wherein the mammalian cells are from a non-human cell line.

82. The method according to claim 80, wherein the mammalian cells are human cells.

83. The method according to any one of the preceding claims, wherein the sample is a body fluid sample, a tissue sample, a fermentation sample, a liquid cultivation sample, a cell culture sample, a water sample, a beverage sample, a pharmaceutical sample, an environmental sample, a sewage sample, a diagnostic sample or a sample of a pharmaceutical product.

84. The method according to claim 83, wherein the body fluid sample is selected from the group consisting of samples from swabs, blood, plasma, serum, urine, bile, saliva, semen, cerebrospinal fluid and mucus.

85. The method according to claim 83, wherein the tissue sample is selected from the group consisting of tissue samples from liver, kidney, muscle, brain, lung, skin, thymus, spleen, gastrointestinal tract, pancreas and thyroid gland.

86. The method according to any one of the preceding claims, wherein the sample is a sample from a human.

87. The method according to any one of the preceding claims, wherein the biological particle is a cell, and the method further comprises isolating cells comprising the target from the substantially liquid compartments.

88. The method according to claim 87, wherein said cells are selected from the group consisting of bacteria, yeast, protozoa, fungus, plant cells, mammalian cells and insect cells, and the target is selected from the group consisting of a nucleic acid, an exosome, a plasmid, an extrachromosomal DNA (ecDNA), an extrachromosomal circular DNA (eccDNA), an aptamer, a nucleic acid tag linked to an aptamer, a molecular beacon, a nucleic acid tag linked to an antibody or a receptor ligand, an antibody, a protein and an enzyme.

89. The method according to any one of claims 87-88, wherein said cells are isolated using a robotics platform.

90. The method according to any one of the preceding claims, further comprising counting the number of particles and the number of particles with reaction product in at least part of the sample.

91. The method according to any one of the preceding claims, further comprising quantifying the amount, such as the concentration, of biological particles and/or target in the sample, by counting the number of spots present in the hydrogel and/or the number of substantially liquid compartments with reaction product.

92. The method according to claim 91, wherein the number of spots and/or substantially liquid compartments with reaction product is counted in an image using image analysis to identify spots and/or the number of substantially liquid compartments with reaction product.

93. The method according to any one of the preceding claims, further comprising recording an image of the hydrogel or mixture before incubation and/or induction of hydrogel formation.

94. The method according to claim 93, wherein said image is used to determine the background of the hydrogel or mixture before incubation.

95. The method according to any one of the preceding claims, wherein multiple images of the hydrogel are recorded while the hydrogel is being incubated, such as wherein images are compared following image processing to determine the number of spots and/or substantially liquid compartments with reaction product.

96. The method of claim 95, wherein the image processing determines the amount of reaction product in each substantially liquid compartment, optionally wherein said amount is correlated to the discrete number of biological particles in each substantially liquid compartment.

97. The method according to claim 96, wherein the multiple images are used to evaluate the formation of reaction product over time.

98. The method according to any one of the preceding claims, wherein the recording of images comprises the use of a confocal scanner, a microscope, a fluorescence microscope, an image cytometer, and/or an automated particle counter.

99. The method according to any one of the preceding claims, wherein the recording of images comprises the use of one or more optical channels.

100. The method according to any one of the preceding claims, wherein the recording of images comprises the use of one or more fluorescent channels.

101. The method according to any one the preceding claims, wherein the images are recorded using an array of detection devices.

102. The method according to any one the preceding claims, wherein the images are recorded using a two-dimensional array of detection devices.

103. The method according to any one of the preceding claims, wherein the images are 3D images.

104. The method according to any one of the preceding claims, wherein the images are recorded using a CCD, a CMOS, a video camera, photomultiplier tubes, photodiode, avalanche photodiode (APD) or a photon counting camera.

105. The method according to any one of the preceding claims, wherein the recorded image is processed.

106. The method according to claim 105, wherein the recorded image is processed using data processing means.

107. The method according to claim 106, wherein the image is processed using artificial intelligence.

108. The method according to any one of claims 105-107, wherein the data processing means distinguish partially overlapping areas with reaction product.

109. The method according to any one of claims 106-108, wherein the data processing means determine or estimate the number of biological particles located in an individual substantially liquid compartment.

110. The method according to claim 109, wherein the determination or estimation of the number of biological particles located in an individual compartment is used to determine the number of targets and/or biological particles in the sample.

111. The method according to any one of claims 106-110, wherein the data processing improves the detection and/or the specificity of the method.

112. The method according to any one of the preceding claims, wherein sample is loaded into a sample compartment, such as wherein the mixture is cured inside said sample compartment.

113. The method according to claim 112, wherein the sample compartment forms part of a cassette.

114. The method according to any one of claims 112-113, wherein the sample compartment is defined by two parallel sheets of transparent material, preferably plastic material or glass material with a given distance between them.

115. The method according to any one of claims 112-114, wherein the cassette comprises channels for loading the sample.

116. The method according to any one of claims 112-115, wherein the cassette comprises channels for flowing the sample within the cassette.

117. The method according to any one of claims 112-116, wherein the cassette comprises a valve for regulating the flow of sample.

118. The method according to any one of claims 112-117, wherein at least one wall of the sample compartment is transparent.

119. The method according to any one of claims 112-118, wherein the cassette is removable.

120. The method according to any one of claims 112-119, wherein the cassette is single-use and/or disposable.

121. The method according to any one of claims 112-120, wherein one or more reagents are preloaded into the sample compartment or into a channel part of the cassette.

122. The method according to any one of the preceding claims, wherein a cellular stain is added to the mixture prior to curing the hydrogel.

123. The method according to claim 122, wherein the cellular stain is a stain which stains the whole cell or a part of the cell, such as the cell membrane, the cell wall, the cytoplasm, the nucleus, the mitochondria and/or nucleic acids.

124. The method according to any one of the preceding claims, further comprising illuminating the hydrogel, whereby cells comprised in the sample are lysed.

125. The method according to any one of the preceding claims, wherein a lysing agent is added to the mixture prior to curing the hydrogel.

126. The method according to any one of the preceding claims, wherein the sample is pre-treated using a method comprising one or more of the following steps:
a. incubating the sample at 98° C. for 5 minutes; and/or
b. mixing the sample with 96% ethanol; and/or
c. mixing the sample with 1-1000 mM sodium hydroxide; and/or
d. mixing the sample with detergent, such as NP40, tween20 or triton X100; and/or
e. mixing the sample with saponin; and/or
f. mixing the sample with 96% methanol.

127. A kit for detecting a biological particle in a sample, said kit comprising:
c. polymer hydrogel reagents; and
d. nucleic acid amplification reagents, the reagents being capable of generating a reaction product in the vicinity of particles having a specific target.

128. The kit according to claim 127, wherein the isothermal amplification reagents comprise reagents as defined in any one of the preceding claims 57-69 and/or wherein the hydrogel components are as defined in any one of the preceding claims 4, 16-20, 32-37, 39-42, and/or 48-49.

129. The kit according to any one of claims 127-128, further comprising a cellular stain.

130. The kit according to any one of claims 127-129, further comprising a lysing agent.

131. The kit according to any one of claims 127-130, further comprising reagents for polymerase chain reaction (PCR), multiple displacement amplification (MDA), strand displacement amplification (SDA), multiple annealing and looping based amplification cycles (MALBAC) or primary template assisted amplification (PTA).

132. A system for detecting a biological particle in a sample, said system comprising:
a. a holder for a cassette with a sample compartment,
b. image forming means capable of forming an image of at least part of the sample in the sample compartment on image acquisition means,
c. image processing means,
d. at least one illumination source capable of illuminating the sample in the sample compartment; and
e. thermostatically controlled heating means capable of heating and optionally cooling the sample in the sample compartment.

133. The system according to claim 132, wherein at least one illumination source is capable of providing specific energy of at least 0.001 J/cm²-500 J/cm², such as 0.01 to 400 J/cm², for example 0.1 to 300 J/cm², such as 1 to 100 J/cm², for example 10-50 J/cm².

134. The system according to any one of claims 132-133, wherein the illumination source is capable of providing electromagnetic radiation with a wavelength of 100 nm-1,200 nm, for example 100 nm-1,000 nm, such as 150 nm-850 nm, for example 200 nm-800 nm, such as 300 nm-750 nm, for example 350 nm-700 nm, such as 350 nm-600 nm, for example 375 nm-500 nm, such as 390 nm-400 nm.

135. The system according to any one of claims 132-134, wherein one illumination source is capable of inducing hydrogel formation, and at least one other illumination source is capable of illuminating the sample to generate an image of at least part of the sample on the image acquisition means.

136. The system according to any one of claims 132-135, wherein the system comprises at least two illumination sources, such as at least 3, 4, 5, 6, 7, or 8 illumination sources, such as excitation light sources, bright field light sources, such as for example a UV light source.

137. The system according to any of claims 132-136, wherein the image forming means is capable of forming a bright field image, a dark field image, and/or a phase contrast image.

138. The system according to any of claims 132-137, wherein the heating means comprise a heating device configured for heating the sample to a temperature of 40-70° C.

139. The system according to any one of the claims 132-138, further comprising means for generating an illumination pattern on the sample, such as a mask, for example a photolithographic mask or a laser.

140. The system according to any one of claims 132-138, wherein the biological particle is a cell, and the system further comprises means for identifying and isolating cells comprising the target from the substantially liquid compartments.

141. The system according to claim 140, wherein said cells are selected from the group consisting of bacteria, yeast, protozoa, fungus, plant cells, mammalian cells and insect cells, and the target is selected from the group consisting of a nucleic acid, an exosome, a plasmid, an extrachromosomal DNA (ecDNA), an extrachromosomal circular DNA (eccDNA), an aptamer, a nucleic acid tag linked to an aptamer, a molecular beacon, a nucleic acid tag linked to an antibody or a receptor ligand, an antibody, a protein and an enzyme.

142. The system according to any one of claims 140-141, wherein the system comprises a robotic platform for isolating cells from the substantially liquid compartments.

143. A cassette for analyzing biological particles comprising a sample compartment having two parallel sheets of transparent material with a give distance between them, wherein the sample compartment or any other part of the cassette comprises hydrogel reagents pre-loaded in dry or freeze-dried form.

144. The cassette of claim 143, further comprising nucleic acid amplification reagents pre-loaded in dry form into the cassette.

145. The cassette of claim 143 or 144, wherein the isothermal amplification reagents comprise reagents as defined in any one of the preceding claims 57-69 and/or wherein the hydrogel components are as defined in any one of the preceding claims 4, 16-20, 32-37, 39-42, and/or 48-49.

146. The cassette of any one of claims 143 to 145, wherein the interior of the sample compartment, has an average thickness of between 20 μm and 2000 μm, between 20 μm and 1000 μm, or between 20 μm and 200 μm.

147. The cassette of any one of claims 143 to 146, wherein the sample compartment has dimensions, in a direction substantially parallel to a wall of an exposing window, in the range between 1 mm by 1 mm and 100 mm by 100 mm, such as 10 mm by 10 mm.

148. The cassette of any one of claims 143 to 147, wherein the exposing window has an area of 0.01 mm² or more, preferably with an area of 0.1 mm² or more, more preferably with an area of 1 mm² or more, preferably with an area of 2 mm² or more, preferably with an area of 4 mm² or more, preferably with an area of 10 mm² or more, preferably with an area of 20 mm² or more, preferably with an area of 40 mm² or more, more preferably with an area of 100 mm² or more, preferably with an area of 200 mm² or more, preferably with an area of 400 mm² or more, preferably with an area of 1000 mm² or more, preferably with an area of 2000 mm2 or more, preferably with an area of 4000 mm² or more, preferably with an area of 10000 mm² or more.

149. The cassette of any one of claims 143 to 148, wherein the cassette further comprises various parts, such as channels and/or valves, which may be used for loading the sample and/or channels for flowing the sample within the cassette, or a valve for regulating the flow of the sample.

150. The cassette of any one of the claims 143 to 149, wherein the volume of the liquid sample, which can be analysed by an external array of detection elements is in the range between 0.01 μL and 20 μL, such as between 0.05 μL and 5 μL.


SEQUENCE LISTING (23 sequences)

SEQ ID NO: 1 - DNA, 18 bp, Artificial sequence
  primer
gtggtggacagccacatg

SEQ ID NO: 2 - DNA, 16 bp, Artificial sequence
  primer
agatccggtggagccg

SEQ ID NO: 3 - DNA, 17 bp, Artificial sequence
  primer
ggcggaaggcgaacatg

SEQ ID NO: 4 - DNA, 18 bp, Artificial sequence
  primer
cccattgccttcgccaga

SEQ ID NO: 5 - DNA, 39 bp, Artificial sequence
  primer
  misc_feature (1)..(1): Fluorescein amidites (FAM) tag
tgtgcagctcctccacgcttttgccatccaccccagcat

SEQ ID NO: 6 - DNA, 45 bp, Artificial sequence
  primer
ctgggcatcgtggagtaccagttttggcagaattggacgactgag

SEQ ID NO: 7 - DNA, 13 bp, Artificial sequence
  primer
  misc_feature (13)..(13): Black Hole Quencher 1 (BHQ-1) tag
gaggagctgcaca

SEQ ID NO: 8 - DNA, 20 bp, Artificial sequence
  primer
gcggccaacttacttctgac

SEQ ID NO: 9 - DNA, 20 bp, Artificial sequence
  primer
caacgttgttgccattgcta

SEQ ID NO: 10 - DNA, 20 bp, Artificial sequence
  primer
tgaagccataccaaacgacg

SEQ ID NO: 11 - DNA, 22 bp, Artificial sequence
  primer
tgttgtgcaaaaaagcggttag

SEQ ID NO: 12 - DNA, 41 bp, Artificial sequence
  primer
  misc_feature (1)..(1): Fluorescein amidites (FAM) tag
aggcgagttacatgatcccccagatcggaggaccgaaggag

SEQ ID NO: 13 - DNA, 38 bp, Artificial sequence
  primer
tgatcgttgggaaccggagccaggcatcgtggtgtcac

SEQ ID NO: 14 - DNA, 13 bp, Artificial sequence
  primer
  misc_feature (13)..(13): Black Hole Quencher 1 (BHQ-1) tag
atgtaactcgcct

SEQ ID NO: 15 - DNA, 39 bp, Artificial sequence; identical to SEQ ID NO: 5
  primer

SEQ ID NO: 16 - DNA, 22 bp, Artificial sequence
  primer
  misc_feature (1)..(1): Black Hole Quencher 1 8BHQ-1) tag
  misc_feature (4)..(4): 1′,2′-Dideoxyribose modification (dspacer)
  misc_feature (6)..(6): Fluorescein amidites (FAM) tag
agaccccattgccttcgccaga

SEQ ID NO: 17 - DNA, 22 bp, Artificial sequence
  primer
  misc_feature (1)..(1): Black Hole Quencher 2 (BHQ-2) tag
  misc_feature (4)..(4): 1′,2′-Dideoxyribose modification (dspacer)
  misc_feature (6)..(6): Cy5 (5′-Cyanine 5)
agatcccattgccttcgccaga

SEQ ID NO: 18 - DNA, 60 bp, Artificial sequence
  primer
cacggcagaattggacgactgagcgcgggatctggcgaaggcaatggggatcttgaaggc

SEQ ID NO: 19 - DNA, 56 bp, Artificial sequence
  primer
acaggtaccgtggtggacagccacatgcacttcaagagcgccatccaccccagcat

SEQ ID NO: 20 - DNA, 59 bp, Artificial sequence
  primer
agaattcagatccggtggagccgggtccggcggtgccgtccacggcagaattggacgac

SEQ ID NO: 21 - DNA, 243 bp, Artificial sequence
  pcr product
acaggtaccgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccattgccttcgccagatcccgcgctcagtcgtccaattctgccgtggacggcaccgccggacccggctccaccggatctgaattc

SEQ ID NO: 22 - DNA, 243 bp, Artificial sequence
  pcr product
acaggtaccgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagatccccattgccttcgccagatcccgcgctcagtcgtccaattctgccgtggacggcaccgccggacccggctccaccggatctgaattc

SEQ ID NO: 23 - DNA, 22 bp, Artificial sequence
  primer
  misc_feature (1)..(1): 5′-biotin tag
acgagaaccccttcctgcacgc